SEQUENCE LISTING (12 sequences)

SEQ ID NO: 1 - DNA, 624 bp, Pyrodictium abyssi
gtgaagtacacaaccctagctatagcgggtattattgcctcggctgccgccctcgccctcctagcaggcttcgccaccacccagagccccctcaacagcttctacgccaccggtacagcacaggcagtaagcgagccaatagacgtagaaagccacctcggcagcataacccccgcagccggcgcacagggcagtgacgacataggttacgcaatagtgtggataaaggaccaggtcaatgatgtaaagctgaaggtgaccctgcgtaacgctgagcagctaaagccctacttcaagtacctacagatacagataacaagcggctatgagacgaacagcacagctctaggcaacttcagcgagaccaaggctgtgataagcctcgacaaccccagcgccgtgatagtactagacaaggaggatatagcagtgctctatccggacaagaccggttacacaaacacttcgatatgggtacccggtgaacctgacaagataattgtctacaacgagacaaagccagtagctatactgaacttcaaggccttctacgaggctaaggagggtatgctattcgacagcctgccagtgatattcaacttccaggtgctacaagtaggctaa

SEQ ID NO: 2 - protein, 207 aa, Pyrodictium abyssi
VKYTTLAIAGIIASAAALALLAGFATTQSPLNSFYATGTAQAVSEPIDVESHLGSITPAAGAQGSDDIGYAIVWIKDQVNDVKLKVTLRNAEQLKPYFKYLQIQITSGYETNSTALGNFSETKAVISLDNPSAVIVLDKEDIAVLYPDKTGYTNTSIWVPGEPDKIIVYNETKPVAILNFKAFYEAKEGMLFDSLPVIFNFQVLQVG

SEQ ID NO: 3 - DNA, 513 bp, Pyrodictium abyssi
gtgaagcctacggctctagccctggctggtatcattgcctcggctgccgacctcgccctgctagcaggcttcgccaccacccagagcccgctcaacagcttctacgccaccggcacagcagccgcaacaagcgagccaatagacgtagagagccacctcagcagcatagcccctgctgctggcgcacagggcagccaggacataggctacttcaacgtgaccgccaaggatcaagtgaacgtgacaaagataaaggtgaccctggctaacgctgagcagctaaagccctacttcaagtacctacagatagtgctaaagagcgaggtagctgacgagatcaaggccgtaataagcatagacaagcctagcgccgtcataatactagacagccaggacttcgacagcaacaacagagcaaagataagcgccactgcctactacgaggctaaggagggcatgctattcgacagcctaccgctaatattcaacatacaggtgctaagcgtcagctaa

SEQ ID NO: 4 - protein, 170 aa, Pyrodictium abyssi
VKPTALALAGIIASAADLALLAGFATTQSPLNSFYATGTAAATSEPIDVESHLSSIAPAAGAQGSQDIGYFNVTAKDQVNVTKIKVTLANAEQLKPYFKYLQIVLKSEVADEIKAVISIDKPSAVIILDSQDFDSNNRAKISATAYYEAKEGMLFDSLPLIFNIQVLSVS

SEQ ID NO: 5 - DNA, 537 bp, Pyrodictium abyssi
atgaggtacacgaccctagctctggccggcatagtggcctcggctgccgccctcgccctgctagcaggcttcgccacgacccagagcccgctaagcagcttctacgccaccggcacagcacaagcagtaagcgagccaatagacgtagagagccacctagacaacaccatagcccctgctgccggtgcacagggctacaaggacatgggctacattaagataactaaccagtcaaaagttaatgtaataaagctgaaggtgactctcgctaacgccgagcagctaaagccctacttcgactacctacagctagtactcacaagcaacgccactggcaccgacatggttaaggctgtgctaagcctcgagaagcctagcgcagtcataatactagacaacgatgactacgatagcactaacaagatacagctaaaggtagaagcctactatgaggctaaggagggcatgctattcgacagcctaccagtaatactgaacttccaggtactgagcgccgcttgcagtcccttgtggtga

SEQ ID NO: 6 - protein, 178 aa, Pyrodictium abyssi
MRYTTLALAGIVASAAALALLAGFATTQSPLSSFYATGTAQAVSEPIDVESHLDNTIAPAAGAQGYKDMGYIKITNQSKVNVIKLKVTLANAEQLKPYFDYLQLVLTSNATGTDMVKAVLSLEKPSAVIILDNDDYDSTNKIQLKVEAYYEAKEGMLFDSLPVILNFQVLSAACSPLW

SEQ ID NO: 7 - DNA, 395 bp, Pyrodictium abyssi
agcttctacgccaccggcacagcacaggcagtaagcgagccaatagacgtggtaagcagcctcggtacgctaaatactgccgctggtgcacagggtaagcagacgctaggagacataacaatatatgcgcacaatgacgtgaacataacaaagctaaaggtcacgcttgctaacgctgcacagctaagaccatacttcaagtacctgataataaagctagtaagcctggacagcaacggcaacgagtccgaggaaaagggcatgataactctatggaagccttacgccgtgataatactagaccatgaagatttcaacaacgacatcgacaatgacggcaacaatgacgccaagataagggttgtagcctactatgaggctaaggagggtatgct

SEQ ID NO: 8 - protein, 131 aa, Pyrodictium abyssi
SFYATGTAQAVSEPIDVVSSLGTLNTAAGAQGKQTLGDITIYAHNDVNITKLKVTLANAAQLRPYFKYLIIKLVSLDSNGNESEEKGMITLWKPYAVIILDHEDFNNDIDNDGNNDAKIRVVAYYEAKEGM

SEQ ID NO: 9 - DNA, 372 bp, Pyrodictium abyssi
agcttctacgccaccggcacagcagaggcaacaagcgagccaatagacgttgtaagcaaccttaacacggccatagcccctgctgccggcgcccagggcagcgtgggcataggcagcataacaatagagaacaagactgacgtgaacgttgtgaagctgaagataaccctcgccaacgctgagcagctaaagccctacttcgactacctacagatagtgctaaagagcgttgacagcaacgagatcaaggctgtgctaagcctcgagaagcccagcgcagtcataatactggacaacgaggacttccagggcggcgacaaccagtgccagatagacgccaccgcctactacgaggctaaggagggtatgcta

SEQ ID NO: 10 - protein, 124 aa, Pyrodictium abyssi
SFYATGTAEATSEPIDVVSNLNTAIAPAAGAQGSVGIGSITIENKTDVNVVKLKITLANAEQLKPYFDYLQIVLKSVDSNEIKAVLSLEKPSAVIILDNEDFQGGDNQCQIDATAYYEAKEGML

SEQ ID NO: 11 - DNA, 448 bp, Artificial Sequence
  consensus sequence
tgagaccctagctgcggattgcctcggctgccgcctcgccctctagcaggcttcgccacacccagagcccctacagcttctacgccaccggcacagcacaggcagtaagcgagccaatagacgtagaaagccacctcacacatagcccctgctgccggcgcacagggcagcaggacataggctacataaaataacaagatagtgaacgtataaagctgaaggtgaccctgctaacgctgagcagctaaagccctacttcaagtacctacagatagtgctaaaagcgacagcaggcacacgagaaggcgtgataagcctcgagaagcctagcgccgtcataatactagacaacgaggacttcgaagcacaacagaaagagaagcaatagcctactacgaggctaaggagggtatgctattcgacagcctcctatataactcaggtctgt

SEQ ID NO: 12 - protein, 140 aa, Artificial Sequence
  consensus sequence
VKTLALAGIIASAALALLAGFATTQSPLSFYATGTAQAVSEPIDVESHLSIAPAAGAQGSDIGYIIKVNVVKLKVTLANAEQLKPYFKYLQIVLSSEIKAVISLDKPSAVIILDEDFAIAYYEAKEGMLFDSLPVINQVL

(19) United States
(12) Patent Application Publication
(10) Pub. No.: US 2005/0208121 A1
(43) Pub. Date: Sep. 22, 2005

(54) Polymeric cannulae proteins, nucleic acids encoding them and methods for making and using them

(76) Inventors: Nelson R. Barton, San Diego, CA (US); Eileen O'Donoghue, San Diego, CA (US); Gerhard Frey, San Diego, CA (US); Jay M. Short, Del Mar, CA (US); W. Michael Lafferty, Encinitas, CA (US); Kevin Chow, Issaquah, WA (US)

Correspondence Address: DIVERSA C/O MOFO S.D., 3811 VALLEY CENTER DRIVE, SUITE 500, SAN DIEGO, CA, 92130, US

(21) Appl. No.: 11/046,203

(22) Filed: Jan. 28, 2005

Related U.S. Application Data
(63) Continuation of application No. 10/370,370, filed on Feb. 18, 2003, now abandoned.
(63) Continuation of application No. 09/997,807, filed on Nov. 30, 2001.
(60) Provisional application No. 60/357,406, filed on Feb. 15, 2002.
(60) Provisional application No. 60/250,426, filed on Nov. 30, 2000.

(51) Int. Cl. A01K 67/00; C07H 21/04; A61K 33/44; C07K 14/705; A61K 38/17

(52) U.S. Cl. 424/450; 530/350; 435/69.1; 435/320.1; 435/325; 536/23.5; 800/8; 800/288; 424/125

(57) ABSTRACT

The invention provides chimeric cannulae polypeptides and methods for making and using them. In one aspect, the invention provides compositions and methods for the identification, separation or synthesis of proteins or ligands. In one aspect, the invention provides compositions and methods for making and using nanotubules. In one aspect, the invention provides compositions and methods for the selection and purification of chiral compositions from racemic mixtures. The invention provides compositions comprising polymers prepared by self-assembly of a plurality of monomeric polypeptide units, including nanorobots, biochips, drug delivery systems. In one aspect, the polymer can form a nanotube, and the polymer (nanotubule) can encapsulate a drug molecule—a drug delivery systems of the invention. A drug delivery system of the invention may be delivered to a particular location of human body to effectively cure a disease or treat a symptom. In one aspect, a targeting vector can be attached to a monomeric polypeptide unit or a polymer to facilitate the targeting of the drug delivery system of the invention.

### RELATED APPLICATIONS

This application is a continuation of U.S. patent application Ser. No. 10/370,370, filed Feb. 18, 2003, which claims the benefit of priority under 35 U.S.C. § 119(e) of U.S. Provisional Application No. 60/357,406, filed Feb. 15, 2002; and, U.S. Ser. No. 09/997,807, filed Nov. 30, 2001, which claims the benefit of priority under 35 U.S.C. § 119(e) of U.S. Provisional Application No. 60/250,426, filed Nov. 30, 2000. The aforementioned applications are explicitly incorporated herein by reference in their entirety and for all purposes.

### TECHNICAL FIELD

This invention relates to pharmacology and drug synthesis. In one aspect, the invention provides compositions and methods for the identification, separation or synthesis of proteins or ligands. In one aspect, the invention provides compositions and methods for making and using nanotubules. In one aspect, the invention provides compositions and methods for the selection and purification of chiral compositions from racemic mixtures. In one aspect, the invention provides chimeric cannulae polypeptides and methods for making and using them. The present invention also relates to the field of producing protein polymers through self-assembly of monomeric polypeptide units and to various uses of the self-assembled protein polymers and compositions comprising these polymers.

### BACKGROUND

Nanotechnology is taking center stage in efforts to build the next generation of computational tools and medical devices. The ability to rearrange molecular structures will have a profound effect on how products are manufactured. However, one drawback to synthetic nanostructures constructed from materials such as carbon and silicon has been the difficulty in attaining self-assembly of such components. Nanobiotechnology relates to the development and use of biomolecular structures for applications such as biochips, drug delivery, data storage and nanomachinery. Nature produces molecular machinery that out-performs anything mankind currently knows how to construct with conventional manufacturing technology.

One application for nanobiotechnology is targeted drug delivery. The major goal of targeted drug delivery is the local accumulation and increased bioavailability of a therapeutic agent at its intended site of action, thereby reducing the drug dosage required to illicit the desired response. These sites of action include pathogenic bacteria and viruses, cancer cells, and areas of inflammation or other tissue damage. There are a variety of targeted drug delivery systems that are currently being developed and these include: liposomes, soluble polymer carriers, lipid and polymer gels, and various nanosuspensions.

Targeted drug delivery systems that utilize encapsulation are attractive because 1) they require lower doses of therapeutic than non-targeted, even biodistribution approaches; 2) the therapeutic is less likely to cause unwanted side effects in healthy tissues because it remains concentrated, isolated, and therefore protected, until delivery; and 3) large numbers of therapeutic molecules can be delivered to a site of action using few targeting vectors attached to the encapsulation vessel.

One recent development in the area of nanotechnology employs eukaryotic microtube assemblies as a structural framework. Eukaryotic microtubules self-assemble into hollow rods and this property has made them attractive candidate structural components for a variety of nanotechnology applications. However, the use of eukaryotic microtubules presents numerous challenges, including the lability of microtubule subunit proteins, the requirement for GTP for microtubule assembly and the need for microtubule stabilizing drugs like taxol to prevent the depolymerization of the tubules below 37° C. or in the presence of calcium. In addition, a major drawback of eukaryotic microtubules is the inability to overexpress microtubule subunits in E. coli in a functional form and therefore microtubule protein must be isolated from a native source, most commonly bovine brain.

In addition, substrates for delivery of biocatalysts for synthesis reactions are needed. Such substrates may be three-dimensional to provide more catalytic sites and, as a result, it may be advantageous to develop such substrates from self-assembling polymers. Also, three-dimensional polymeric structures may be useful for other applications such as separation processes or screening methods.

Enantiomers frequently display dramatically different pharmacological properties. As a result, use of single-enantiomer drugs may improve efficacy and reduce side effects. The United States Food and Drug Administration also recognizes the importance of understanding the pharmacological properties of each enantiomer. In order for a racemic drug to be registered, the biological activity of each purified enantiomer must be characterized.

A unique feature of all of members of the genus Pyrodictium is the formation of an extracellular network comprising hollow protein tubes, the so-called cannulae nanotubules. The cells are bound into this network. These fiber networks are a unique feature of the genus Pyrodictium and they appear to be required for growth above 100° C. Originally these hollow protein tubes were called “fibers,” but to distinguish them from eukaryotic microtubules they were re-named “cannulae” (derived from the Latin: canna, tube; diminutive form: cannula, small tube). See, e.g., “In Vitro Untersuchungen zum extrazellularen Netzwerk von Pyrodictium abyssi TAG11”, 1998, Biologie und Medizin, University of Regensburg, Naturwissenschaftlichen Fakultat III.

Cannulae nanotubules, such as CanA, CanB, CanC, CanD or CanE including, e.g., the exemplary polypeptides SEQ ID NO:2, SEQ ID NO:4, SEQ ID NO:6, SEQ ID NO:8 or SEQ ID NO:10, are characteristically formed by Pyrodictium abyssi, a hyperthermophilic microorganism discovered in a high temperature environment (>100° C.). In its natural environment and in cell culture, Pyrodictium abyssi are linked together by a meshwork of these nanotubular fibers that both connect and entrap the cells. There appears to be a direct association between the maintenance of these nanotubular connections and cellular growth as demonstrated by the observation that, at the onset of cellular fission, these nanotubules appear to form loops attached at both ends to the growing cell. Following cellular fission the nanotubular loops become links connecting daughter cells. While it remains speculative as to what the true role of the nanotubules is in nature, it has been suggested that the linkage of cells by these tubules could enable cells to exchange metabolites, genetic information, or signal compounds.

Cannulae A, or CanA, is a heat-resistant protein capable of forming nanotubules. CanA nanotubules are assembled from 21 kDa monomeric subunits that self-assemble in the presence of divalent cation into hollow rods with an outer diameter of approximately 25 nm and an inner diameter of approximately 20 nm, thus exhibiting molecular dimensions and an overall morphology not dissimilar to eukaryotic microtubules. CanA monomer expressed in E. coli is heat-stable. It can be rapidly purified from bacterial extracts following heat treatment to remove the majority of the heat-labile host proteins. Following purification, the CanA monomer readily self-assembles into nanotubules in the presence of calcium and magnesium at elevated temperature. The assembled nanotubule structure contains 28 CanA monomers per turn arranged with a helical pitch. The CanA nanotubules are heat stable (up to 128° C.) and remain assembled in the presence of SDS or high concentrations of urea.

### SUMMARY

The invention provides nanoscale drug delivery vehicles, which in one aspect are for targeted drug delivery, and methods for making them. The invention also provides compositions made from, or comprising, a self-assembled protein polymer, e.g., a cannulae protein, such as chimeric polypeptides of the invention, which in alternative aspects comprise a CanA polypeptide as set forth in SEQ ID NO:2 (encoded by, e.g., SEQ ID NO:1); a CanB polypeptide as set forth in SEQ ID NO:4 (encoded by, e.g., SEQ ID NO:3); a CanC polypeptide as set forth in SEQ ID NO:6 (encoded by, e.g., SEQ ID NO:5); a CanD polypeptide as set forth in SEQ ID NO:8 (encoded by, e.g., SEQ ID NO:7); a CanE polypeptide as set forth in SEQ ID NO:10 (encoded by, e.g., SEQ ID NO:9), or subsequences thereof, or sequence substantially identical thereto, or a combination thereof. These compositions can be drug delivery vehicles, biomedical devices, analytical devices, and the like, such as liposomes, fibers, nanoscopic screws, nanorobots, biochips, three-dimensional arrays and the like. In another aspect, the invention provides a medium for biocatalysts based on a self-assembled protein polymer, wherein the polymer comprises chimeric polypeptides of the invention, or composition of the invention, which in alternative aspects comprises a CanA polypeptide as set forth in SEQ ID NO:2 (encoded by, e.g., SEQ ID NO:1); a CanB polypeptide as set forth in SEQ ID NO:4 (encoded by, e.g., SEQ ID NO:3); a CanC polypeptide as set forth in SEQ ID NO:6 (encoded by, e.g., SEQ ID NO:5); a CanD polypeptide as set forth in SEQ ID NO:8 (encoded by, e.g., SEQ ID NO:7); a CanE polypeptide as set forth in SEQ ID NO:10 (encoded by, e.g., SEQ ID NO:9), or sequence substantially identical thereto, or a combination thereof.

In one aspect, the invention provides methods of producing a self-assembled protein polymer (including compositions of the invention, e.g., drug delivery vehicles, fibers, biochips) including the steps of: providing a plurality of polypeptides having a sequence comprising SEQ ID NO:2, SEQ ID NO:4, SEQ ID NO:6, SEQ ID NO:8, or SEQ ID NO:10, and sequences substantially identical (defined below) thereto; and amino acid sequences encoded by a nucleic acid having a sequence as set forth in SEQ ID NOS: 1, 3, 5, 7, and 9, sequences substantially identical (defined below) thereto and sequences complementary thereto; and inducing self-assembly of the plurality of polypeptides to form the polymer.

In one aspect, the invention provides methods of encapsulating a material including the steps of: dissolving a plurality of polypeptides having a sequence selected from the group consisting of SEQ ID NO:2, SEQ ID NO:4, SEQ ID NO:6, SEQ ID NO:8, or SEQ ID NO:10, subsequences thereof, and sequences substantially identical thereto; and amino acid sequences encoded by SEQ ID NOS: 1, 3, 5, 7, and 9, sequences substantially identical thereto and sequences complementary thereto; and the material in a solution; and polymerizing the plurality of polypeptides to form a polymer in the presence of the material in solution so as to encapsulate the material in the polymer.

In one aspect, the invention provides drug delivery systems comprising at least one drug encapsulated in a self-assembled protein polymer made from a chimeric polypeptide of the invention, or, a plurality of polypeptides having a sequence selected from the group consisting of SEQ ID NO:2, SEQ ID NO:4, SEQ ID NO:6, SEQ ID NO:8, or SEQ ID NO:10, subsequences thereof, or sequences substantially identical thereto; and amino acid sequences encoded by SEQ ID NOS: 1, 3, 5, 7, and 9, sequences substantially identical thereto and sequences complementary thereto.

In one aspect, the invention provides methods of generating a variant including the steps of: obtaining a nucleic acid having a sequence selected from the group consisting of SEQ ID NOS: 1, 3, 5, 7, and 9, sequences substantially identical thereto, sequences complementary thereto, fragments having at least 30 consecutive nucleotides of SEQ ID NOS:1, 3, 5, 7, and 9, and fragments having at least 30 consecutive nucleotides of the sequences complementary to SEQ ID NOS: 1, 3, 5, 7, 9; and modifying one or more nucleotides in the sequence to another nucleotide, deleting one or more nucleotides in the sequence, or adding one or more nucleotides to the sequence to generate a variant.

In one aspect, the invention provides assays for identifying functional polypeptide fragments or variants encoded by fragments of SEQ ID NOS: 1, 3, 5, 7, and 9, and sequences substantially identical thereto, which retain the enzymatic function of the polypeptides of SEQ ID NO:2, SEQ ID NO:4, SEQ ID NO:6, SEQ ID NO:8, or SEQ ID NO:10, and sequences substantially identical thereto. The assay includes the steps of: dissolving a plurality of polypeptides of SEQ ID NOS: 2, 4, 6, 8 and 10, and sequences substantially identical thereto, or polypeptide fragments or variants encoded by SEQ ID NOS: 1, 3, 5, 7 and 9, sequences substantially identical thereto, and sequences substantially complementary thereto in a solution containing a template molecule and alkaline earth metal ion; and detecting the presence of a polymer in the solution by analyzing the solution using a method selected from High Performance Liquid Chromatography (HPLC), Gel Permeation Chromatography (GPC) and light scattering.

In one aspect, the invention provides polypeptides comprising: a chimeric polypeptide of the invention, or, a sequence selected from the group consisting of SEQ ID NO:2, SEQ ID NO:4, SEQ ID NO:6, SEQ ID NO:8, or SEQ ID NO:10, subsequences thereof, sequences substantially identical thereto, and amino acid sequences encoded by SEQ ID NOS: 1, 3, 5, 7, 9, sequences substantially identical thereto and sequences complementary thereto, and a functional group covalently attached to the sequence, wherein the side group comprises a structure selected from the group consisting of an antibody, an oligosaccharide, a polynucleotide, a polyethylene glycol and a charged group.

The invention provides methods of producing a polypeptide polymer comprising the steps of: (a) providing a plurality of monomeric polypeptides and at least one divalent cation, wherein the monomer polypeptides are capable of self-assembly in the presence of a divalent cation; and (b) (i) polymerizing the monomeric polypeptides through a self-assembly process in the presence of at least one divalent cation, or, (ii) polymerizing the monomeric polypeptides in the presence of a template molecule, under conditions wherein the monomeric polypeptides self-assemble, thereby producing a polypeptide polymer, wherein each monomeric polypeptide of the plurality of monomeric polypeptides have either (a) an amino acid sequence as set forth in SEQ ID NO:2, SEQ ID NO:4, SEQ ID NO:6, SEQ ID NO:8, or SEQ ID NO:10, or, (b) an amino acid sequence as set forth in SEQ ID NO:2, SEQ ID NO:4, SEQ ID NO:6, SEQ ID NO:8, or SEQ ID NO:10, having at least one conservative substitution, and at least one monomeric polypeptide of the plurality of monomeric polypeptides includes a modification comprising attachment of an enzyme, attachment of a nucleotide or attachment of a nucleotide derivative, or attachment of a lipid or attachment of a lipid derivative, or attachment of a targeting molecule, or attachment of a vector. In one aspect, the step of providing a plurality of monomeric polypeptides further comprises the steps of: preparing a vector comprising a nucleic acid, wherein the nucleic acid encodes the monomeric polypeptide; inserting the vector into a host cell; growing the host cell in a suitable culture to express the nucleic acid to form the polypeptide; and isolating the formed monomeric polypeptide from the host cell. In one aspect, the step of polymerizing the monomeric polypeptides further comprises the steps of: dissolving the plurality of monomeric polypeptides in a solution; and adding a template molecule and an alkaline earth metal ion to the solution. In one aspect, the vector is selected from the group consisting of viral vectors, plasmid vectors, phage vectors, phagemid vectors, cosmids, fosmids, bacteriophages, artificial chromosomes, adenovirus vectors, retroviral vectors, and adeno-associated vectors. In one aspect, the host is selected from the group consisting of prokaryotes, eukaryotes, funguses, yeasts, plants and metabolically rich hosts.

In one aspect, the monomeric polypeptides have a molecular weight of more than 5,000 daltons, or, a molecular weight of more than 10,000 daltons. In one aspect, the monomeric polypeptides polymerize to form a hollow tube, a tubule, a micelle or a molecular sieve. In one aspect, the hollow tube has approximately a 25 nm outer diameter and a 20 nm inner diameter. In one aspect, the monomeric polypeptides are polymerized in the presence of a divalent cation and a template molecule. In one aspect, the template molecule comprises a plasmid, a phage, a cosmid, a phagemid, a virus or a portion of a virus. In one aspect, the virus comprises a retrovirus, a parainfluenzavirus, a herpesvirus, a reovirus or a paramyxovirus. In one aspect, the portion of a virus comprises a coat protein, a spike glycoprotein or a capsid protein.

In one aspect, the plurality of monomeric polypeptides are polymerized in the presence of at least one divalent cation selected from the group consisting of Ca²⁺, Mg²⁺, Cu²⁺, Zn 2+, Sr²⁺, Ni²⁺, Mn²⁺ and Fe²⁺. In one aspect, the plurality of monomeric polypeptides are polymerized in the presence of Ca²⁺ and Mg²⁺. In one aspect, the step of polymerizing the monomeric polypeptides further comprises the step of dissolving the monomeric polypeptides in an aqueous solution.

In one aspect, the template molecule is prepared by fragmenting or shearing of a suspension of a polymer. In one aspect, the monomeric polypeptides interact with each other by pairing, bundling, entangling or electrostatic cross-linking, thereby generating paired polymers, bundled polymers, entangled polymers, cross-linked polymers or an interconnected network of polymers.

In one aspect, the method further comprises providing a therapeutic agent or a drug molecule and adding the therapeutic agent or drug molecule to the polymerization step, thereby generating a therapeutic agent or drug molecule encapsulated by the polymers. In one aspect, the therapeutic agent or drug molecule is added to the polymerization step.

In one aspect, the method further comprising capping the partially formed polymer using a capping unit, e.g., wherein the capping unit comprises a polypeptide monomer. In one aspect, the therapeutic agent or drug encapsulating step is carried out by mixing the polymer and the therapeutic agent or drug molecule together in a solution such that the therapeutic agent or drug molecule can permeate inside the polymer. In one aspect, the method further comprises attaching a targeting molecule or a vector to the therapeutic agent- or drug-loaded polymer during the encapsulation process or after the completion of the encapsulation process. In one aspect, the method further comprises using lipids or lipid molecules during the encapsulation process. In one aspect, the method further comprises attaching the polymer to a hydrogel, e.g., wherein the hydrogel comprises a three-dimensional structural network for a biochip. In one aspect, the monomeric polypeptide has an amino acid sequence having 55%, 60%, 65%, 70%, 75%, 80%, 85%, 90%, 95%, 98%, 99%, or more, or complete sequence identity to an amino acid sequence as set forth in exemplary SEQ ID NO:2, SEQ ID NO:4, SEQ ID NO:6, SEQ ID NO:8, or SEQ ID NO:10, or, an amino acid sequence as set forth in SEQ ID NO:2, SEQ ID NO:4, SEQ ID NO:6, SEQ ID NO:8, or SEQ ID NO:10, with at least one conservative amino acid substitution, e.g., wherein the conservative amino acid substitution comprises substituting one amino acid for another of the same class, such as substitution of one hydrophobic amino acid for another, or substitution of one polar amino acid for another, or substitution of isoleucine, valine, leucine or methionine, for another hydrophobic amino acid, or substitution of arginine for lysine, glutamic acid for aspartic acid or glutamine for asparagine (i.e., sequences substantially identical to SEQ ID NO:2, SEQ ID NO:4, SEQ ID NO:6, SEQ ID NO:8, or SEQ ID NO:10).

In one aspect of the method of the invention for producing a polypeptide polymer, the polypeptide polymer comprises a nanoscale delivery vehicle. In one aspect, the at least one monomeric polypeptide further comprises an enzyme, or, a nucleotide or a nucleotide derivative, or, a lipid or a lipid derivative, or, a vector or a targeting molecule, such as an TM antibody, an oligosaccharide, a MORPHATIDE™.

The invention provides chimeric polypeptides comprising at least a first domain comprising a cannulae polypeptide and at least a second domain comprising a heterologous polypeptide or peptide. The heterologous polypeptide or peptide can be inserted at the amino terminal end, the carboxy terminal end or internal to the cannulae polypeptide, or, if the cannulae polypeptide comprises more than one heterologous polypeptide or peptide, a mixture thereof. The cannulae polypeptide can comprise a protein having at least about 55%, 60%, 65%, 70%, 75%, 80%, 85%, 90%, 95%, 98%, 99%, or more, sequence identity to SEQ ID NO:2, SEQ ID NO:4, SEQ ID NO:6, SEQ ID NO:8, or SEQ ID NO:10 (i.e., sequences substantially identical to exemplary cannulae proteins of the invention, SEQ ID NO:2, SEQ ID NO:4, SEQ ID NO:6, SEQ ID NO:8, or SEQ ID NO:10), and is capable of assembling into a polymer, e.g., a nanotubule, or, is capable of acting as a chiral selector. The chimeric cannulae proteins can assemble into nanotubular polymers to act as chiral selectors, biosynthetic pathways, selection scaffoldings and the like.

In one aspect, the cannulae polypeptide is capable of assembling into a polymer, such as a nanotubule. In one aspect, the cannulae polypeptide is capable of self-assembling into a polymer. In some aspects, the monomers require a co-factor for polymer assembly, e.g., a divalent cation, or, a “nucleation factor,” which can be another cannulae monomer. The divalent cation can be Ca²⁺, Mg²⁺, Cu²⁺, Zn²⁺, Sr²⁺, Ni²⁺, Mn²⁺ and/or Fe²⁺. In another aspect, both Ca²⁺ and Mg²⁺ are needed for polymer assembly, e.g., into nanotubules. In one aspect, divalent cation(s) are in millimolar concentrations during polymer assembly.

In one aspect, the heterologous polypeptide or peptide is expressed in the inner lumen of a nanotubule or on the exterior of the nanotubule. These hybrid nanotubules can array the heterologous polypeptides or peptides on the outer surface or the inner luminal surface of a tubular polymer, or, when a monomer comprises more than one heterologous peptide or protein, they can be “displayed” on both the outer and inner surfaces of the tubules. If all the monomers of a nanotubule comprise a heterologous polypeptide or peptide in a similar manner, then that heterologous polypeptide or peptide can be displayed in a regular helical pattern on the nanotubule.

In one aspect, the heterologous polypeptide or peptide comprises a chiral selection motif, a receptor or a ligand, an enzyme, an enzyme active site, a cofactor, a substrate, an antigen or an antigen binding site, a detectable moiety, e.g., a green fluorescent protein, an alpha-galactosidase or a selection factor, e.g., a chloramphenicol acetyltransferase.

In one aspect, the chimeric polypeptide is a recombinant protein, which can be expressed in vitro or in vivo, a synthetic protein, or a mixture thereof.

In one aspect, at least one subsequence of the cannulae polypeptide domain of a chimeric protein of the invention has been removed. A heterologous polypeptide or peptide can be inserted into the cannulae polypeptide at the site (or one of the sites) subsequence(s) were removed. In one aspect, the cannulae polypeptide is a CanA polypeptide and the removed subsequence is a 14 residue motif (peptide) consisting of residue (position) 123 to residue 136 of SEQ ID NO:2 (i.e., “PDKTGYTNTSIWVP”), or, a 17 residue motif (peptide) located at amino acid residue (position) 123 to residue 139 of SEQ ID NO:2, (i.e., “PDKTGYTNTSIWVPGEP”). The heterologous polypeptide or peptide can be inserted into the CanA polypeptide at one or both of the sites of the 14 or 17 residue motif subsequences that were removed. The heterologous peptide can be a 14 residue or a 17 residue peptide inserted into the CanA polypeptide to replace the removed 14 residue or 17 residue motif.

The invention provides immobilized chimeric polypeptides comprising a chimeric monomeric or polymeric polypeptide of the invention. The invention provides polymers, e.g., nanotubules, comprising a plurality of chimeric polypeptides of the invention. In one aspect, the polymer is a heteropolymer, e.g., a nanotubule assembled from more than one cannulae polypeptide, including monomers other than the chimeric proteins of the invention, or other polypeptides or compositions. The heterologous polypeptide or peptide comprises an enzyme, e.g., an active site, or a plurality of different enzymes. The plurality of enzymes can comprise a biosynthetic pathway. The plurality of enzymes can be arranged along the length of the nanotubule in the same order as they act in the biosynthetic pathway. The different enzymes comprising the biosynthetic pathway can be separated from each other along the length of the tubule by cannulae monomers lacking a heterologous protein or peptide (e.g., a “wild type” cannulae monomer, such as CanA, CanB, CanC, CanD or CanE including, e.g., the exemplary polypeptides SEQ ID NO:2, SEQ ID NO:4, SEQ ID NO:6, SEQ ID NO:8 or SEQ ID NO:10, and the like). The polymers comprising a biosynthetic pathway can also comprise substrate(s), co-factor(s), regulatory agents and the like.

The invention provides polymers, e.g., nanotubules, wherein the heterologous polypeptide or peptide comprises at least one chiral selection motif, such as an enzyme or an enzyme active site.

The invention provides nucleic acids comprising a sequence encoding a chimeric polypeptide of the invention. The invention provides expression cassettes (e.g., vectors, recombinant viruses, phages, etc.) comprising a sequence encoding a chimeric polypeptide of the invention. The invention provides cells comprising a sequence encoding a chimeric polypeptide of the invention, or, an expression cassette of the invention. The cell can be any cell, e.g., a bacterial cell, a plant cell, a yeast cell, a fungal cell, an insect cell or a mammalian cell. The invention provides transgenic non-human animals comprising a sequence encoding a chimeric polypeptide of the invention, or, an expression cassette of the invention. The invention provides plants comprising a sequence encoding a chimeric polypeptide of the invention, or, an expression cassette of the invention.

The invention provides methods for the chiral selection of a composition, comprising the following steps: providing a chimeric polypeptide of the invention; providing a racemic mixture of the composition; and, contacting the racemic mixture with the chimeric polypeptide under conditions wherein only one enantiomer of the composition binds to the chimeric polypeptide; thereby selecting a single chiral specie of the racemic mixture. The invention provides methods for the chiral selection of a composition, comprising the following steps: providing a nanotubule of the invention; providing a racemic mixture of the composition; and, contacting the racemic mixture with the nanotubule under conditions wherein only one enantiomer of the composition binds to the nanotubule; thereby selecting a single chiral specie of the racemic mixture. The methods further comprise separation of the different chiral species.

The invention provides methods for enzymatic biosynthesis of a composition, comprising the following steps: providing a nanotubule of the invention comprising a plurality of enzymes comprising a biosynthetic pathway; providing a substrate for at least one enzyme; and, contacting the nanotubule with the substrate under conditions wherein the enzymes of the biosynthetic pathway catalyze the synthesis of the composition. In one aspect, the enzymes are expressed in the inner lumen of the nanotubule, or, they are expressed on the exterior of the nanotubule. The nanotubules can also comprise substrates(s), co-factor(s), regulatory factors and the like.

The invention provides chimeric polypeptides comprising at least a first domain comprising a cannulae polypeptide and a second domain comprising a heterologous polypeptide or peptide, wherein the cannulae polypeptide is capable of assembling into a polymer, e.g., assembles into a nanotubule. In one aspect, the cannulae polypeptide is capable of acting as a chiral selector, e.g., comprises a chiral selection motif and is capable of acting as a chiral selector. In one aspect, cannulae polypeptide comprises a CanA, CanB, CanC, CanD or CanE including, e.g., the exemplary polypeptides SEQ ID NO:2, SEQ ID NO:4, SEQ ID NO:6, SEQ ID NO:8 or SEQ ID NO:10, subsequences thereof or a combination thereof. In one aspect, the chiral selection motif comprises, or is derived from, serum albumin, α₁-acid glycoprotein, avidin, ovomucoid, transferrin, cytochrome c, lysozyme, pepsin, cellulase or cellobiohydrolase. In one aspect, the heterologous polypeptide or peptide comprises an epitope, an N-terminal identification peptide, or a detection or purification facilitating domain (such as a metal chelating peptide or a protein A domain or a FLAG™ (a FLAG sequence of amino acids) extension/affinity purification system, or a cleavable linker sequence. In one aspect, the metal chelating peptide comprises a polyhistidine tract or a histidine-tryptophan module. In one aspect, the cleavable linker sequence comprises a Factor Xa domain or an enterokinase domain.

The invention provides chimeric polypeptides comprising at least a first domain comprising a cannulae polypeptide and a second domain comprising an enzyme substrate, an enzyme cofactor, or a selection factor. The invention provides chimeric polypeptides comprising at least a first domain comprising a cannulae polypeptide and a second domain comprising a heterologous polypeptide or peptide, wherein the heterologous polypeptide or peptide comprises a detectable moiety, such as a green fluorescent protein, an alpha-galactosidase or a selection factor. In one aspect, the selection factor comprises a chloramphenicol acetyltransferase. In one aspect, the polymer comprises a higher order structure, e.g., a nanotubule. In one aspect, the polymer self-assembles into a higher order structure.

The invention provides chimeric polypeptides comprising at least a first domain comprising a cannulae polypeptide and a second domain comprising a heterologous polypeptide or peptide, wherein the cannulae polypeptide comprises a protein having 70%, 80%, 90% or 95% sequence identity (i.e., sequences substantially identical to the exemplary cannulae proteins) or complete sequence identity to SEQ ID NO:2, SEQ ID NO:4, SEQ ID NO:6, SEQ ID NO:8, or SEQ ID NO:10, and acts as a chiral selector, wherein only one enantiomer of a racemic composition binds to the chimeric polypeptide.

The invention provides polymers comprising a chimeric polypeptide, wherein the chimeric polypeptide comprises at least a first domain comprising a cannulae polypeptide and a second domain comprising a heterologous polypeptide or peptide, made by a method comprising: incubating a plurality of chimeric polypeptides with a divalent cation or a nucleation factor under conditions wherein the chimeric polypeptides self-assemble into the polymer. In one aspect, the method comprises incubating the chimeric polypeptides with Ca²⁺, Mg²⁺, Cu²⁺, Zn²⁺, Sr²⁺, Ni²⁺, Mn²⁺ and/or Fe²⁺. In one aspect, the nucleation factor comprises a monomeric chimeric polypeptide. In one aspect, the cannulae polypeptide comprises a protein having 70%, 80%, 90% or 95% (i.e., sequences substantially identical to the exemplary cannulae proteins SEQ ID NO:2, SEQ ID NO:4, SEQ ID NO:6, SEQ ID NO:8, or SEQ ID NO:10) or complete sequence identity to SEQ ID NO:2, SEQ ID NO:4, SEQ ID NO:6, SEQ ID NO:8, or SEQ ID NO:10, and assembles into a polymer.

The invention provides drug delivery systems comprising: a polymeric encapsulation medium made by self-assembly of a plurality of polypeptides; and, at least one drug encapsulated in said polymeric encapsulation medium. In one aspect the drug delivery system comprises a targeting vector, e.g., a polypeptide such as an antibody, or another molecule. In one aspect each of the plurality of polypeptides has at least 50%, 60%, 70%, 80%, 90% or 95% or complete (100%) homology (sequence identity) to a polypeptide having a sequence selected from the group consisting of SEQ ID NO:2, SEQ ID NO:4, SEQ ID NO:6, SEQ ID NO:8 and SEQ ID NO:10 (i.e., sequences substantially identical to SEQ ID NOs:2, 4, 6, 8 and 10), which can be determined by analysis with a sequence comparison algorithm or by visual inspection. In one aspect, each of the plurality of polypeptides comprises at least 10 consecutive amino acids of a polypeptide having a sequence selected from the group consisting of SEQ ID NOs:2, 4, 6, 8, and 10, or sequences substantially identical thereto. For example, in one aspect, each of the plurality of polypeptides is encoded by a nucleic acid comprising a sequence selected from the group consisting of SEQ ID NOS: 1, 3, 5, 7 and 9; variants (sequences substantially identical thereto) encoding self-assembly proteins having at least about 50%, 60%, 70%, 80%, 90% or 95% homology to SEQ ID NOS: 1, 3, 5, 7 and 9 over a region of at least about 100 or 200 residues or the complete (entire) sequence, as determined by analysis with a sequence comparison algorithm or by visual inspection; sequences complementary to SEQ ID NOS: 1, 3, 5, 7 and 9 or variants; and, nucleic acids that hybridize to nucleic acids having any of the foregoing sequences under conditions of low, moderate or high stringency. In one aspect, each of the plurality of polypeptides is encoded by a first nucleic acid, which hybridizes to a second nucleic acid under conditions of low, moderate or high stringency.

The invention provides methods of producing a polypeptide polymer by self-assembly comprising the steps of: providing a plurality of polypeptides capable of self-assembly in the presence of a divalent cation; and polymerizing the polypeptides in the presence of a divalent cation and a template molecule. In one aspect, each of the plurality of self-assembly polypeptides is encoded by a nucleic acid comprising a sequence selected from the group consisting of SEQ ID NOS: 1, 3, 5, 7 and 9, or having a sequence SEQ ID NOS:2, 4, 6, 8 and 10; variants (sequences substantially identical thereto) having at least about 50%, 60%, 70%, 80%, 90% or 95% homology to SEQ ID NOS: 1, 3, 5, 7 and 9, or SEQ ID NOS:2, 4, 6, 8 and 10, over a region of at least about 100 or 200 residues or the complete (entire) sequence. In one aspect, each of the plurality of self-assembly polypeptides is encoded by nucleic acids that hybridize to nucleic acids having any of the foregoing sequences under conditions of low, moderate or high stringency.

In one aspect, the method further comprises the step of providing a plurality of polypeptides further comprises the steps of: preparing a vector with a nucleic acid attached, wherein the nucleic acid encodes the polypeptide; inserting the vector into a host cell; growing the host cell in a suitable culture to express the nucleic acid to form the polypeptide; and, isolating the formed polypeptide from the host cell. In one aspect, the method of polymerizing the polypeptides further comprises the steps of: dissolving the plurality of polypeptides in a solution; and, adding a template molecule and alkaline earth metal ions to the solution. In one aspect, the vector comprises plasmid pEX-CAN-A. In one aspect, the host cell is selected from the group consisting of E. Coli BL21 (DE3) and Pseudomonas.

The invention provides methods of delivering a drug to a location in the human or animal body comprising the step of: administering a drug delivery system of the invention to a human or animal body. In one aspect, the method further comprises the step of releasing the drug from the delivery system at the location in the human or animal body. In one aspect, the method further comprises the steps of: dissolving the plurality of polypeptides and the drug in a solution; and, polymerizing the plurality of polypeptides in the presence of the drug so as to encapsulate the drug in the polymer to form the drug delivery system.

The invention provides methods of encapsulating a molecule comprising the steps of: providing a solution of a plurality of self-assembling polypeptides having a sequence selected from the group consisting of SEQ ID NOS: 2, 4, 6, 8 and 10, and sequences substantially identical thereto (e.g., having at least 50%, 60%, 70%, 80%, 90% or 95% homology to a sequence selected from SEQ ID NOS: 2, 4, 6, 8 and 10), which in one aspect is determined by analysis with a sequence comparison algorithm or by visual inspection; and, polymerizing the plurality of polypeptides the presence of the molecule so as to encapsulate the molecule in the polymer. In one aspect, at least one of said polypeptides comprises a target vector.

The invention provides methods of encapsulating a molecule comprising the steps of: providing a solution of a plurality of polypeptides, wherein each polypeptide is encoded by a nucleic acid comprising a sequence selected from the group consisting of SEQ ID NOS: 1, 3, 5, 7 and 9, variants having at least about 50%, 60%, 70%, 80%, 90% or 95% homology to SEQ ID NOS: 1, 3, 5, 7 and 9 over a region of at least about 100 residues, as determined by analysis with a sequence comparison algorithm or by visual inspection, sequences complementary to SEQ ID NOS: 1, 3, 5, 7 and 9, and sequences complementary to variants having at least about 50%, 60%, 70%, 80%, 90% or 95% homology to SEQ ID NOS: 1, 3, 5, 7 and 9 over a region of at least about 100 residues, as determined by analysis with a sequence comparison algorithm or by visual inspection, and isolated nucleic acids that hybridize to nucleic acids having any of the foregoing sequences under conditions of low, moderate and high stringency; and, polymerizing the plurality of polypeptides the presence of the molecule so as to encapsulate the molecule in the polymer. In one aspect, at least one of said polypeptides comprises a target vector.

The invention provides methods of generating a variant of a nucleic acid encoding a self-assembling protein, comprising: obtaining a nucleic acid comprising a sequence selected from the group consisting of SEQ ID NOS: 1, 3, 5, 7 and 9, variants having at least about 50%, 60%, 70%, 80%, 90% or 95% homology to SEQ ID NOS: 1, 3, 5, 7 and 9 over a region of at least about 100 residues, as determined by analysis with a sequence comparison algorithm or by visual inspection, sequences complementary to SEQ ID NOS: 1, 3, 5, 7 and 9, sequences complementary to variants having at least about 50% homology to SEQ ID NOS: 1, 3, 5, 7 and 9 over a region of at least about 100 residues, as determined by analysis with a sequence comparison algorithm or by visual inspection, and isolated nucleic acids that hybridize to nucleic acids having any of the foregoing sequences under conditions of low, moderate and high stringency, and fragments comprising at least 30 consecutive nucleotides of any of the foregoing sequences; and modifying said sequence by one or more steps selected from the group consisting of modifying one or more nucleotides in said sequence to another nucleotide, deleting one or more nucleotides in said sequence, and adding one or more nucleotides to said sequence. In one aspect, the modifications are introduced by a method selected from the group consisting of error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, Gene Site Saturation Mutagenesis™ (GSSM™) and any combination thereof. In one aspect, at least one modification is made to a codon of the polynucleotide.

The invention provides assays for identifying functional polypeptide fragments or variants (of self-assembling proteins) encoded by fragments of SEQ ID NOS: 1, 3, 5, 7, and 9, and sequences having at least about 50%, 60%, 70%, 80%, 90% or 95% homology to SEQ ID NOS: 1, 3, 5, 7 and 9 over a region of at least about 100 residues, as determined by analysis with a sequence comparison algorithm or by visual inspection, which retain at least one property of the polypeptides of SEQ ID NOS: 2, 4, 6, 8, and 10, and sequences having at least about 50%, 60%, 70%, 80%, 90% or 95% homology to SEQ ID NOS: 2, 4, 6, 8 and 10, over a region of at least about 100 residues, as determined by analysis with a sequence comparison algorithm or by visual inspection, said assay comprising the steps of: providing a solution of a plurality of polypeptides having a sequence selected from the group consisting of SEQ ID NOS: 2, 4, 6, 8, and 10, and sequences having at least about 50%, 60%, 70%, 80%, 90% or 95% homology to SEQ ID NOS: 2, 4, 6, 8 and 10 over a region of at least about 100 residues, as determined by analysis with a sequence comparison algorithm or by visual inspection, polypeptide fragments or variants encoded by SEQ ID NOS: 1, 3, 5, 7, and 9, sequences having at least about 50%, 60%, 70%, 80%, 90% or 95% homology to SEQ ID NOS: 1, 3, 5, 7 and 9 over a region of at least about 100 residues, as determined by analysis with a sequence comparison algorithm or by visual inspection, and sequences complementary to any of the foregoing sequences, in a solution containing a template molecule and alkaline earth metal ion; and, detecting a presence of a polymer in the solution. In one aspect, the step of detecting the presence of a polymer in the solution is carried out by analyzing the solution using a method selected from HPLC, GPC and light scattering.

The invention provides polypeptides comprising: a sequence selected from the group consisting of SEQ ID NOS: 2, 4, 6, 8 and 10, sequences having at least 50%, 60%, 70%, 80%, 90% or 95% or complete homology to a sequence selected from SEQ ID NOS: 2, 4, 6, 8, and 10, as determined by analysis with a sequence comparison algorithm or by visual inspection; and at least one functional group selected from the group consisting of a heterologous protein or peptide, an antibody, an oligosaccharide or polysaccharide, a polynucleotide, and a polyethylene glycol.

The invention provides polypeptides comprising: an amino acid sequence encoded by a sequence selected from the group consisting of SEQ ID NOS: 1, 3, 5, 7 and 9, variants having at least about 50%, 60%, 70%, 80%, 90% or 95% or complete homology to SEQ ID NOS: 1, 3, 5, 7 and 9 over a region of at least about 100 residues, as determined by analysis with a sequence comparison algorithm or by visual inspection, sequences complementary to SEQ ID NOS: 1, 3, 5, 7 and 9, and sequences complementary to variants having at least about 50%, 60%, 70%, 80%, 90% or 95% or complete homology to SEQ ID NOS: 1, 3, 5, 7 and 9 over a region of at least about 100 residues, as determined by analysis with a sequence comparison algorithm or by visual inspection, and isolated nucleic acids that hybridize to nucleic acids having any of the foregoing sequences under conditions of low, moderate and high stringency, and at least one functional group selected from the group consisting of a heterologous protein or peptide, an antibody, an oligosaccharide, a polynucleotide, and a polyethylene glycol.

The invention provides nucleic acid probes comprising an oligonucleotide from about 10 to 50 nucleotides in length and having a segment of at least 10 contiguous nucleotides that is at least 50%, 60%, 70%, 80%, 90% or 95% or complete complementary to a nucleic acid target region of the nucleic acid sequence selected from the group consisting of SEQ ID NOS: 1, 3, 5, 7 and 9, and which hybridizes to the nucleic acid target region under moderate to highly stringent conditions to form a detectable target:probe duplex. In one aspect, the oligonucleotide is DNA or RNA. In one aspect, the oligonucleotide is 15-50 bases in length. In one aspect, the probe further comprises a detectable isotopic label, such as a fluorescent molecule, a chemiluminescent molecule, an enzyme, a cofactor, an enzyme substrate or a hapten. In one aspect, the nucleic acid probe comprises an oligonucleotide from about 15 to 50 nucleotides in length and having a segment of at least 15 contiguous nucleotides that is at least 90%, 95% or 97%, or complete complementary to a nucleic acid target region of the nucleic acid sequence selected from the group consisting of SEQ ID NOS: 1, 3, 5, 7 and 9, and which hybridizes to the nucleic acid target region under moderate to highly stringent conditions to form a detectable target:probe duplex.

The invention provides separation agents comprising a polymer made by self-assembly of a plurality of polypeptides has at least 50%, 60%, 70%, 80%, 90% or 95% or complete homology to a polypeptide having a sequence selected from the group consisting of SEQ ID NOS: 2, 4, 6, 8 and 10, as determined by analysis with a sequence comparison algorithm or by visual inspection.

The invention provides methods of isolating a chiral compound from a mixture comprising the steps of: providing a polymeric separation agent comprising a polymer of the invention, e.g., comprising a chimeric polypeptide of the invention; and eluting the mixture containing the chiral compound through the resin to achieve a separation of the chiral compound from rest material in the mixture.

The invention provides fibers comprising a polymer made by self-assembly of a plurality of polypeptides, e.g., chimeric polypeptides of the invention, or, polypeptides having at least 50%, 60%, 70%, 80%, 90% or 95% or complete homology to a polypeptide having a sequence selected from the group consisting of SEQ ID NOS: 2, 4, 6, 8 and 10.

The invention provides lubricants, or coating compositions, comprising a polymer made by self-assembly of a plurality of polypeptides, wherein each of the plurality of polypeptides comprises chimeric polypeptides of the invention, or, polypeptides having at least 50%, 60%, 70%, 80%, 90% or 95% or complete homology to a polypeptide having a sequence selected from the group consisting of SEQ ID NOS: 2, 4, 6, 8 and 10.

The invention provides biochips comprising a polymer made by self-assembly of a plurality of polypeptides, wherein each of the plurality of polypeptides comprises chimeric polypeptides of the invention, or, polypeptides having at least 50%, 60%, 70%, 80%, 90% or 95% or complete homology to a polypeptide having a sequence selected from the group consisting of SEQ ID NOS: 2, 4, 6, 8 and 10.

The invention provides nanomechanical components, e.g., optical switches or optical waveguides, comprising a polymer made by self-assembly of a plurality of polypeptides, wherein each of the plurality of polypeptides comprises chimeric polypeptides of the invention, or, polypeptides having at least 50%, 60%, 70%, 80%, 90% or 95% or complete homology to a polypeptide having a sequence selected from the group consisting of SEQ ID NOS: 2, 4, 6, 8 and 10.

The invention provides nanorobots comprising a polymer made by self-assembly of a plurality of polypeptides, wherein each of the plurality of polypeptides comprises chimeric polypeptides of the invention, or, polypeptides having at least 50%, 60%, 70%, 80%, 90% or 95% or complete homology to a polypeptide having a sequence selected from the group consisting of SEQ ID NOS: 2, 4, 6, 8 and 10.

The invention provides a computer readable medium having stored thereon a nucleic acid sequence selected from the group consisting of SEQ ID NOS: 1, 3, 5, 7 and 9, variants having at least about 50% homology to SEQ ID NOS: 1, 3, 5, 7 and 9 over a region of at least about 100 residues, as determined by analysis with a sequence comparison algorithm or by visual inspection, sequences complementary to SEQ ID NOS: 1, 3, 5, 7 and 9, and sequences complementary to variants having at least about 50% homology to SEQ ID NOS: 1, 3, 5, 7 and 9 over a region of at least about 100 residues, as determined by analysis with a sequence comparison algorithm or by visual inspection, and isolated nucleic acids that hybridize to nucleic acids having any of the foregoing sequences under conditions of low, moderate and high stringency. The invention provides a computer system comprising a processor and a data storage device wherein said data storage device has stored thereon a nucleic acid sequence selected from the group consisting of SEQ ID NOS: 1, 3, 5, 7 and 9, variants having at least about 50% homology to SEQ ID NOS: 1, 3, 5, 7 and 9 over a region of at least about 100 residues, as determined by analysis with a sequence comparison algorithm or by visual inspection, sequences complementary to SEQ ID NOS: 1, 3, 5, 7 and 9, and sequences complementary to variants having at least about 50% homology to SEQ ID NOS: 1, 3, 5, 7 and 9 over a region of at least about 100 residues, as determined by analysis with a sequence comparison algorithm or by visual inspection, and isolated nucleic acids that hybridize to nucleic acids having any of the foregoing sequences under conditions of low, moderate and high stringency. In one aspect, the invention further comprising a sequence comparison algorithm and a data storage device having at least one reference sequence stored thereon. In one aspect, the sequence comparison algorithm comprises a computer program which indicates polymorphisms. In one aspect, the computer system further comprises an identifier which identifies one or more features in said sequence.

The invention provides a method for comparing a first sequence to a second sequence comprising the steps of: reading the first sequence and the second sequence through use of a computer program which compares sequences; and, determining differences between the first sequence and the second sequence with the computer program, wherein said first sequence is a nucleic acid sequence selected from the group consisting of SEQ ID NOS: 1, 3, 5, 7 and 9, variants having at least about 50% homology to SEQ ID NOS: 1, 3, 5, 7 and 9 over a region of at least about 100 residues, as determined by analysis with a sequence comparison algorithm or by visual inspection, sequences complementary to SEQ ID NOS: 1, 3, 5, 7 and 9, and sequences complementary to variants having at least about 50% homology to SEQ ID NOS: 1, 3, 5, 7 and 9 over a region of at least about 100 residues, as determined by analysis with a sequence comparison algorithm or by visual inspection, and isolated nucleic acids that hybridize to nucleic acids having any of the foregoing sequences under conditions of low, moderate and high stringency. In one aspect, the step of determining differences between the first sequence and the second sequence further comprises the step of identifying polymorphisms. The invention provides a method for identifying a feature in a particular sequence comprising the steps of: reading the particular sequence using a computer program which identifies one or more features in a sequence; and, identifying one or more features in the particular sequence with the computer program, wherein the particular sequence selected from the group consisting of SEQ ID NOS: 1, 3, 5, 7 and 9, variants having at least about 50% homology to SEQ ID NOS: 1, 3, 5, 7 and 9 over a region of at least about 100 residues, as determined by analysis with a sequence comparison algorithm or by visual inspection, sequences complementary to SEQ ID NOS: 1, 3, 5, 7 and 9, and sequences complementary to variants having at least about 50% homology to SEQ ID NOS: 1, 3, 5, 7 and 9 over a region of at least about 100 residues, as determined by analysis with a sequence comparison algorithm or by visual inspection, and isolated nucleic acids that hybridize to nucleic acids having any of the foregoing sequences under conditions of low, moderate and high stringency.

The invention provides protein preparations comprising a polypeptide having an amino acid sequence selected from the group consisting of SEQ ID NOS: 2, 4, 6, 8 and 10, sequences having at least about 50% homology to a sequence selected from the group consisting of SEQ ID NOS: 2, 4, 6, 8 and 10, as determined by analysis with a sequence comparison algorithm, and sequences having at least 10 consecutive amino acid residues of a sequence selected from the group consisting of SEQ ID NOS: 2, 4, 6, 8 and 10.

The invention provides expression vectors capable of replicating in a host cell comprising a polynucleotide having a sequence selected from the group consisting of SEQ ID NOS: 1, 3, 5, 7 and 9, variants having at least about 50% homology to SEQ ID NOS: 1, 3, 5, 7 and 9 over a region of at least about 100 residues, as determined by analysis with a sequence comparison algorithm or by visual inspection, sequences complementary to SEQ ID NOS: 1, 3, 5, 7 and 9, and sequences complementary to variants having at least about 50% homology to SEQ ID NOS: 1, 3, 5, 7 and 9 over a region of at least about 100 residues, as determined by analysis with a sequence comparison algorithm or by visual inspection, and isolated nucleic acids that hybridize to nucleic acids having any of the foregoing sequences under conditions of low, moderate and high stringency. In one aspect, the vector is selected from the group consisting of viral vectors, plasmid vectors, phage vectors, phagemid vectors, cosmids, fosmids, bacteriophages, artificial chromosomes, adenovirus vectors, retroviral vectors, and adeno-associated viral vectors. The invention provides host cells comprising an expression vector of the invention. In one aspect, the host is selected from the group consisting of prokaryotes, eukaryotes, funguses, yeasts, plants and metabolically rich hosts.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is an illustration of a transmission electron micrograph of nanotubules assembled from recombinant CanA expressed in E. coli; i.e., a transmission electron micrograph of one embodiment of a self-assembled protein polymer useful in the present invention.

FIG. 2 is a schematic representation of the open reading frames of the CanA and CanB sequences, showing the CanA sequence containing a 14 amino acid domain not found in CanB.

FIG. 3 is an illustration of an immunofluorescent light microscope image of nanotubules assembled from a fusion protein generated by fusing the CanA open reading frame (SEQ ID NO:1) to the open reading frame of the green fluorescent protein ZSGREEN™.

FIG. 4 is an illustration of an exemplary process for constructing a heteropolymer of the invention generated by self-assembly of different chimeric monomers, as described below.

FIG. 5 diagrammatically illustrates one embodiment of a process for encapsulating a drug in a nanoscale delivery vehicle according to the present invention.

FIG. 6A diagrammatically illustrates a solution containing lipids, monomeric polypeptide units and drug molecules according to the present invention. FIG. 6B diagrammatically illustrates a formed liposome encapsulating monomeric polypeptide units and drug molecules according to the present invention. FIG. 6C diagrammatically illustrates an encapsulated drug composition according to present invention.

FIG. 7 diagrammatically illustrates a process of fusing a heat stable polypeptide of the present invention with an enzyme to form a heat stable enzyme according to the present invention.

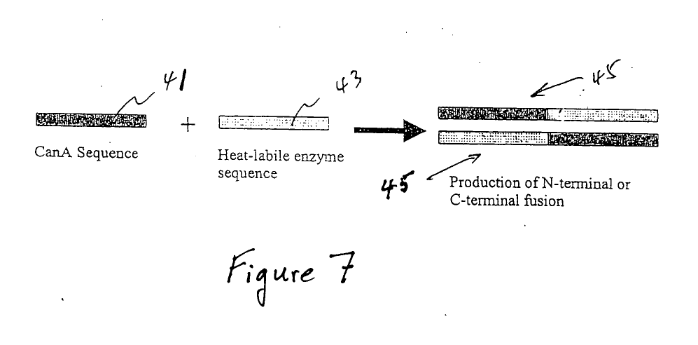

Like reference symbols in the various drawings indicate like elements.

### DETAILED DESCRIPTION

The invention provides compositions and methods for the identification, separation or synthesis of proteins or ligands using chimeric cannulae polypeptides (i.e., fusion, or hybrid, proteins) of the invention, or, polymers of the invention. Chimeric cannulae polypeptides of the invention in alternative aspects comprise a CanA polypeptide as set forth in SEQ ID NO:2 (encoded by, e.g., SEQ ID NO:1); a CanB polypeptide as set forth in SEQ ID NO:4 (encoded by, e.g., SEQ ID NO:3); a CanC polypeptide as set forth in SEQ ID NO:6 (encoded by, e.g., SEQ ID NO:5); a CanD polypeptide as set forth in SEQ ID NO:8 (encoded by, e.g., SEQ ID NO:7); a CanE polypeptide as set forth in SEQ ID NO:10 (encoded by, e.g., SEQ ID NO:9), or subsequences thereof, or sequences substantially identical thereto, or a combination thereof.

In one aspect, the compositions and methods are used for the chiral separation of proteins and other compositions. For example, cannulae (e.g., CanA, CanB, CanC, CanD, CanE) fusion proteins can be used as chiral separations material. The chimeric cannulae polypeptides of the invention can be used as chiral separation materials in monomer or polymer (e.g., nanotubule) forms. When used in nanotubule forms, the motif of the cannulae polypeptide responsible for chiral selectivity can be exposed to the inner lumen of the tubule or on the outer surface of the tubule, or both.

The compositions of the invention, e.g., the polymer of the invention, may be used as (or used in) molecular machine components such as shafts or gears, for nanorobots for a wide variety of applications, including biomedical applications.

The invention provides cannulae (e.g., CanA, CanB, CanC, CanD or CanE including, e.g., the exemplary polypeptides SEQ ID NO:2, SEQ ID NO:4, SEQ ID NO:6, SEQ ID NO:8 or SEQ ID NO:10) fusion proteins comprising a cannulae polypeptide further comprising a heterologous polypeptide or peptide. The heterologous polypeptide or peptide can be an enzyme, an enzyme active site, a ligand, a receptor, an antigen, an epitope, or an antibody. The heterologous polypeptide or peptide can be any sequence for the chiral selection of a protein or other composition. For example, a chiral selection heterologous polypeptide or peptide can be an enzyme or an enzyme active site motif.

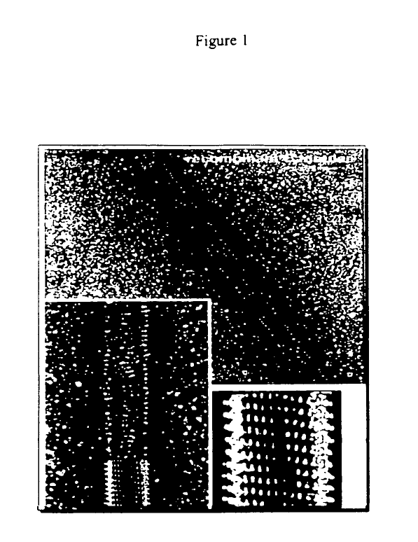

In one aspect, the cannulae fusion proteins are monomeric or polymeric, e.g., dimers, trimers, etc., or nanotubules, as illustrated in FIG. 1. Cannulae chimeric polymers, e.g., nanotubules, can act as a high density preparation materials, e.g., where the heterologous polypeptide or peptide comprise a chiral selection motif.

Cannulae chimeric polymers, e.g., nanotubules, also can act as a high density selection materials, e.g., where the heterologous polypeptide or peptide comprise a receptor, ligand, epitope, antibody and the like. In aspects where the cannulae chimeric polymers form as nanotubules, the heterologous polypeptide or peptide can be expressed on the outer surface of the nanotubule, on the inner surface of the tubule's lumen, or both.

Cannulae chimeric polymers, e.g., nanotubules, also can act as a biosynthetic scaffolding, e.g., where nanotubules of the invention comprise a plurality of heterologous polypeptide or peptides in the form of enzymes, catalytic antibodies or enzyme active sites comprising a biosynthetic pathway. In one aspect, the enzymes, catalytic antibodies or enzyme active sites are all expressed on one surface of a nanotubule, e.g., on the outer surface or on the inner lumen of the tubule. In one aspect, the enzymes, catalytic antibodies or enzyme active sites are arranged along the length of the tubule in the same order of their action in the biosynthetic pathway. Any number of enzymes, catalytic antibodies or enzyme active sites can be immobilized onto a tubule. Any biosynthetic pathway can be reconstructed along a nanotubule of the invention.

In one aspect, nanotubules comprising a plurality enzymes, catalytic antibodies and/or enzyme active sites are generated by constructing a cannulae polypeptide-enzyme fusion protein by fusing the open reading frame of a cannulae polypeptide (e.g., CanA, CanB, CanC, CanD or CanE including, e.g., the exemplary polypeptides SEQ ID NO:2, SEQ ID NO:4, SEQ ID NO:6, SEQ ID NO:8 or SEQ ID NO:10) to the open reading frame of a desired enzyme sequence using standard molecular cloning techniques. The fusion sequence is then cloned into an appropriate expression cassette, e.g., an over-expression vector, prokaryotic or eukaryotic, and expressed as recombinant proteins. Expressed fusion protein can be purified from host proteins before polymer assembly. For example, chimeric proteins (e.g., monomers) can be purified by heat treatment to denature heat-labile host proteins (e.g., at about 80 to 100° C., for about 2 to 20 minutes). The soluble heat-stable fusion protein can be further purified from contaminating proteins by other conventional means, e.g., chromatography techniques, e.g., ion exchange chromatography, HPLC and the like.

Purified, partially purified or unpurified chimeric (fusion) proteins can be induced to assemble into nanotubules by heating the fusion monomer solution (e.g., to about 80° C.) in the presence of millimolar concentrations of a bivalent cation, e.g., calcium and/or magnesium. The polymer can be collected, e.g., by centrifugation (e.g., at 30,000×g for 30 minutes), chromatography and the like.

The invention provides heteropolymers, e.g., nanotubules, comprising any variety of compositions, such as enzymes, catalytic antibodies and/or enzyme active sites, co-factors, substrates and the like to construct a biosynthetic pathway along the length of the polymer (e.g., nanotubule). Heteropolymers (e.g., nanotubules) of the invention can also comprise any variety of antibodies, antigens, receptors, ligands, binding sites and the like, spatially arranged in any desired manner along the length of the polymer.

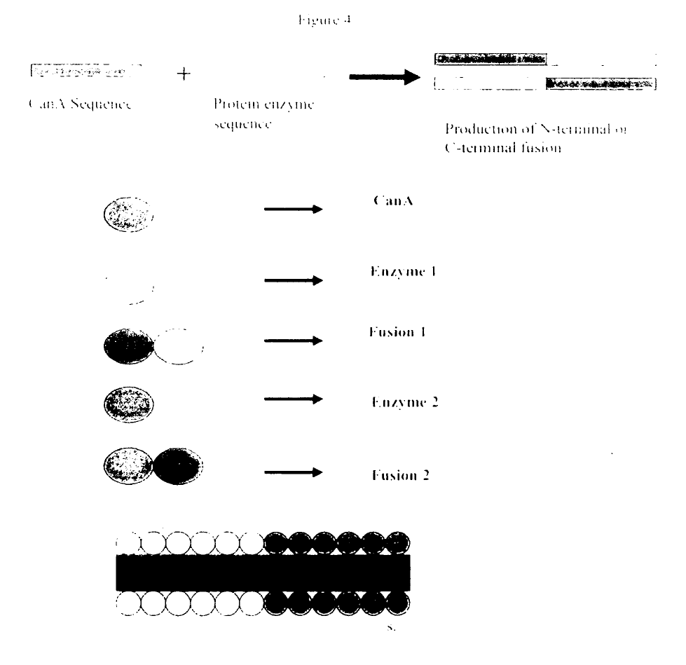

Heteropolymers (e.g., nanotubules comprising a plurality of different enzymes, catalytic antibodies and/or enzyme active sites comprising a biosynthetic pathway) can be constructed by an exemplary protocol as illustrated in FIG. 4. Nucleic acids encoding chimeric monomers are constructed and expressed. The heterologous protein or peptide can be inserted at the amino terminal, carboxy terminal (as shown in FIG. 4) or internal to the cannulae polypeptide (e.g., CanA). One or more, or all, or the expressed chimeric monomers can be purified. Self-assembly of the heteropolymer can be initiated with one of the chimeric polypeptides, e.g., fusion 1 monomer pool as shown in FIG. 4. Next, in this exemplary protocol, fusion 1 polymer is rapidly diluted with fusion 2-monomer pool such that the majority of the subunits added to the growing polymer are fusion 2 monomers. Alternatively, unassembled fusion 1 monomers are removed and fusion 2 monomers added. The resulting polymer is composed of a length of fusion 1 and a length of fusion 2 monomer. This process can be iteratively repeated until a nanotubule of a desired length comprising a desired number of different enzymes, catalytic antibodies and/or enzyme active sites comprising a biosynthetic pathway is generated. The resulting nanotubule can serve as a scaffold for the assembly of an oriented, multi-enzyme complex.

In alternative aspects, the invention provides heteropolymers comprising different ratios of fusions and wild-type, non-fusion monomers to assemble nanotubular polymers that display one or more enzyme (or other, e.g., binding or co-factor) activities, at controlled loading, on the exterior or interior surface of a nanotubule.

In one aspect, any number of compositions desired to be immobilized along the length of a polymer of the invention (e.g., a nanotubule), whether a protein or a non-protein composition, e.g., enzymes, catalytic antibodies, enzyme active sites, co-factors (e.g., NADH, FADH, ATP and the like), substrates, antibodies, antigens, receptors, ligands, binding sites and the like, can be spatially arranged in any desired manner along the length of the polymer by indirect immobilization to the polymer. In this aspect, immobilization agents (e.g., receptors, ligands, antibodies, epitopes, substrates and the like) are arranged as desired along the length of the polymer. The composition to be immobilized can be constructed to include (e.g., a chimeric recombinant protein) or be complexed with a moiety that will bind to an indirect immobilization agent. The indirect immobilization agent can be a binding agent for the composition to be immobilized. For example, a nanotubule is constructed having ten different antibodies spatially arranged along the length of the tubule. This nanotubule can be constructed by a method analogous to that illustrated in FIG. 4, e.g., instead of chimeric monomers comprising enzymes, the chimeric monomers would comprise antibodies (including, e.g., antigen binding sites) that specifically bind to different, desired enzymes, substrates, co-factors and the like.

In one aspect, the chimeric cannulae proteins of the invention self-assemble into helical nanotubular protein polymers. These helical nanotubular protein polymers can act as a chiral selectors, biosynthetic pathways, selection scaffoldings and the like. These hybrid protein nanotubules can array the heterologous polypeptide or peptide (fusion partner) on the outer surface or the inner luminal surface of a tubular polymer. If all the monomers of a nanotubule comprise a heterologous polypeptide or peptide in a similar manner, then that heterologous polypeptide or peptide can be displayed in a regular helical pattern on the nanotubule.

In addition to serving as chiral selectors, biosynthetic pathways, selection scaffoldings, etc. comprising chimeric monomers of the invention, polymers of the invention (e.g., nanotubular protein polymers) can also comprise unmodified cannulae monomers, modified non-chimeric cannulae monomers or other polypeptides. For example, in one aspect, a nanotubule of the invention comprises a chimeric monomer A, an unmodified cannulae monomer, a chimeric monomer B, etc. Inclusion of unmodified cannulae monomers can provide “spacing” between the “clusters” of heterologous peptides or polypeptides expressed on the inner or outer surface of a nanotubules (spatially arranged, e.g., as illustrated in FIG. 4). In one aspect, a polymer of the invention is designed to comprise a mix of proteins having different stabilities under different conditions, e.g., a nanotubule comprising temperature stable and temperature labile monomers (chimeric or wild type, e.g., SEQ ID NO:2, SEQ ID NO:4, SEQ ID NO:6, SEQ ID NO:8, or sequences substantially identical to the exemplary SEQ ID NO:2, SEQ ID NO:4, SEQ ID NO:6, or SEQ ID NO:8). In one aspect, a polymer of the invention is designed to comprise a mix of different proteins, e.g., cannulae polypeptides, including chimeric, wild type or otherwise modified, e.g., non-thermostable.

In one aspect, a subsequence of a cannulae protein is removed and replaced by the heterologous polypeptide or peptide, or, the heterologous polypeptide or peptide can be added to a cannulae monomer. The removed subsequence can be amino- or carboxy-terminal, or, it can be internal to the cannulae protein. In one aspect, the removed subsequence is a motif that is expressed on the inner surface and/or the exterior surface of a cannulae nanotubule. Thus, when the removed sequence is expressed by a heterologous sequence, the heterologous sequence is also expressed on the inner or the outer surface (or both) of the tubule.

In one aspect, for the fusion (hybrid) CanA protein, the removed subsequence consists of a 14 residue motif consisting of residue 123 to residue 136 of SEQ ID NO:2 (i.e., “PDKTGYTNTSIWVP”), or, a 17 residue motif located at amino acid residue 123 to residue 139 of SEQ ID NO:2, (i.e., “PDKTGYTNTSIWVPGEP”). In one aspect, the removed sequence is replaced by a heterologous polypeptide or peptide. When the CanA monomer is in polymeric nanotubular form, a 14 residue motif consisting of residue 123 to residue 136 of SEQ ID NO:2 (i.e., “PDKTGYTNTSIWVP”), or, a 17 residue motif located at amino acid residue 123 to residue 139 of SEQ ID NO:2, (i.e., “PDKTGYTNTSIWVPGEP”) is expressed on the outer surface of the nanotubule. In this aspect, the CanA monomer protein can act as a chiral selector on the outer surface. If all the monomers of a nanotubule comprise a heterologous polypeptide or peptide inserted in or near this motif position (as an addition or a full or partial replacement for the CanA motif), then that heterologous polypeptide or peptide can be displayed in a regular helical pattern on the outer surface of a CanA nanotubule. In one aspect, a 14 residue or a 17 residue heterologous peptide replaces the removed 14 residue motif consisting of residue 123 to residue 136 of SEQ ID NO:2 or the 17 residue motif located at amino acid residue 123 to residue 139 of SEQ ID NO:2.

In one aspect, the chimeric cannulae protein of the invention, either in monomeric or polymer (e.g., nanotubule) form, are stable to a variety of conditions, e.g., temperature, pHs, chaotropic agents, detergents and the like. In one aspect, a polymer of the invention comprises a heteropolymer comprising monomers of different stabilities under different conditions.

In one aspect, the monomers and polymers of the invention are used as chiral selectors, and methods for using these compositions for the chiral selection of compositions from racemic mixtures. The net charge and electrophoretic mobility of a protein chiral selector can be directly affected by the pH of the buffer solution (e.g., aqueous buffers) used during the separation. In one aspect, the separation methods of the invention (e.g., the chiral separation methods using cannulae fusion (hybrid) proteins as a chiral selectors) are practiced over a range of pH values. The pH of the buffer solution for use in the separations methods can be varied and optimal pH can be determined by routine screening. In one aspect, the methods are practiced over an operating range from about pH 5.5 to 8.5, or, pH 3 to pH 10, or, pH 2.5 to pH 11.

In one aspect, the separation methods of the invention (e.g., chiral selections) are practiced over a range of pH values and in the presence of SDS and/or urea. The presence of SDS and/or urea can improve aqueous chiral separations; see, e.g., Bojarski (1997) Electrophoresis 18:965-969. The stability screenings can be conducted as follows: purified recombinant cannulae monomer protein is assembled into polymer using an in vitro assembly protocol at neutral pH. Following completion of the assembly reaction, the sample is centrifuged and pelleted cannulae polymer collected.

The stability of nanotubules comprising cannulae fusion (hybrid) proteins can be affected by the buffer environment used in practicing the methods of the invention. The separation methods of the invention (e.g., the chiral separation methods) can be practiced in a variety of commonly used organic modifiers. In one aspect, organic modifiers are added to buffers used in practicing the methods of the invention to improve the resolution of enantiomers. The concentration of modifiers for use in the separations methods can be varied and optimal concentrations can be determined by routine screening.

In one aspect the invention provides methods to evaluate the stability of the polymers of the invention in the presence of commonly used organic modifiers, e.g., as listed in the following table:

| Organic Modifier | Concentration Range |
|---|---|
| Methanol | 0-15% |
| Ethanol | 0-15% |
| 1-propanol | 0-15% |
| 2-propanol | 0-15% |
| acetonitrile | 0-15% |

These modifiers are organic modifiers commonly used in protein-based chiral selection methods development. The methods of the invention incorporate these and other organic modifiers and protocols as discussed by, e.g., Busch (1993) J. of Chromatography A. 635:119-126; De Lorenzi (1997) J. of Chromatography A. 790:47-64; Ahmed (1997) J. of Chromatography A. 766:237-244. All of the analytical methods used for the evaluation of polymer stability in aqueous buffers also may be compatible with buffers containing up to 15% (v/v) of these organic modifiers. The choice of buffer and buffer pH used for organic modifier screenings can incorporate the results of aqueous buffer stability studies. In one aspect, these modifiers are analyzed in buffers between pH 6.5 and pH 8.0, or, between pH 5.5 and pH 9.0, or between pH 4.5 and pH 10.0.

In one aspect, the chiral selectivity of chimeric cannulae monomer and/or polymers of the invention and the yield of the chiral selection methods of the invention are determined using capillary electrophoretic methods. In one aspect, the chiral selectivity method is evaluated using capillary electrophoretic methods and racemic mixtures of commercially available compositions, e.g., beta-blockers or equivalents. These methods also can be used to evaluate the efficiency (e.g., the chiral selectivity) of various embodiments of the invention, e.g., regular, helical nanotubules comprising chimeric and/or wild type CanA, CanB, CanC, CanD or CanE including, e.g., the exemplary polypeptides SEQ ID NO:2, SEQ ID NO:4, SEQ ID NO:6, SEQ ID NO:8 or SEQ ID NO:10, etc. or mixed species polymers. Data obtained from stability studies also can be used to determine by routine screening optimal buffer pH, acceptable additives, and organic modifier concentrations, depending on the desired outcome of a particular chiral separation protocol.

In one aspect, the resolution obtained with polymers (e.g. nanotubular chimeric cannulae) and monomers of the invention is determined using commercially available chiral selectors. There are numerous published methods for separating racemic mixtures of racemic compositions, e.g., beta-blockers, using commercial chiral selectors with, e.g., capillary electrophoresis. These methods can utilize both protein and non-protein chiral selectors. In one aspect, tests incorporating commercially available enantio-separations media provide data about the comparative efficiency of nanotubular chimeric cannulae polymers and monomers of the invention as chiral selectors.

In one aspect, a chiral selectivity method of the invention or the resolution obtained with a polymer (e.g., nanotubular chimeric cannulae) and/or monomer of the invention is evaluated using capillary electrophoretic methods and racemic mixtures of commercially available beta-blockers, such as, e.g., those listed below:

| Compound | Structure |
|---|---|
| Sotalol | 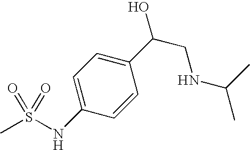 |
| Atenolol | 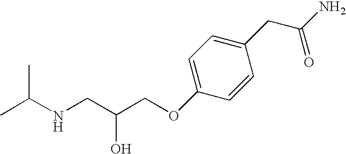 |
| Acebutolol | 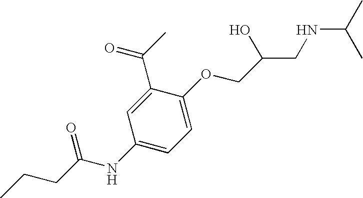 |
| Pindolol | 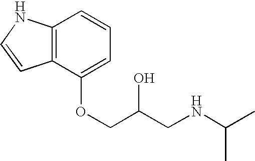 |
| Metoprolol | 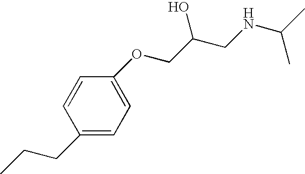 |
| Propranolol | 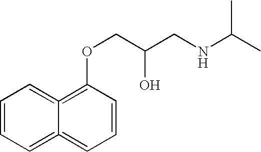 |
| Alprenolol | 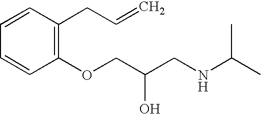 |
| Labetalol | 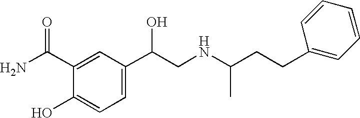 |

In one aspect, a chiral selectivity method of the invention or the resolution obtained with polymers and monomers of the invention is evaluated using capillary electrophoretic methods and racemic mixtures of propanolol. There are numerous reports in the literature that describe the resolution of enantiomers of propanolol, making it a good benchmark for the routine screening for optimizing chiral separations methods conditions employing the compositions of the invention, e.g., chimeric cannulae monomers and polymers (including nanotubules). Enantioseparation of propanolol has been accomplished using quail egg white riboflavin binding protein (see, e.g., De Lorenzi (1997) supra), pepsin, cellobiohydrolase, and bovine serum albumin (see, e.g., Tanaka (2001) J. of Biochem. Biophysical Methods 48:103-116; Henriksson (1996) FEBS Letters 390:339-344).

In one aspect, monomers of polymers of the invention are immobilized on a surface, e.g., a capillary. In one aspect, the methods of the invention are practice in a capillary tube, e.g., a GIGAMATRIX™ (Diversa Corporation, San Diego, Calif.). Both untreated and polyacrylamide-coated capillaries can be used to practice the methods of the invention. Untreated capillaries may be unsuitable for chiral selection due to adsorption of a chiral selector or an analyte on the walls of the capillary, see, e.g., Tanaka (2001) supra.

As discussed above, any separation fluid or organic modifier can be used to practice the methods of the invention. Determining optimal conditions by routine screening can be based on an optimization procedure described by Allenmark, S.G Chromatographic Enantioseparation. Methods and applications. pg 90-141. 1998. West Sussex, England, Ellis Horwood Limited. This exemplary protocol uses a neutral buffer without additives or modifiers as the starting condition for separation. If the enantiomers are not resolved, the pH can be adjusted to pH 5.5 or 8.5. If one of these pH conditions results in loss of sample due to excessive complexation with a chimeric cannulae monomer or polymer of the invention, a low percentage of an organic modifier can be introduced. Changes also can be made to the buffer pH, choice of organic modifier, and concentration of organic modifier to improve resolution.

In one aspect, routine screening methods are carried out using a partial filling technique, as described, e.g., by Tanaka (2001) supra; Chankvetadze (2001) J. of Chromatography A. 906:309-363. In this exemplary technique the capillary (e.g., GIGAMATRIX™, Diversa Corporation, San Diego, Calif.) is only partially filled with the protein chiral selector (a chimeric cannulae monomer or polymer of the invention). This can minimize the sensitivity issues associated with the high UV backgrounds produced by protein at the detector. Using this method, it is possible to use up to 500 uM protein during the enantioseparation.

A countercurrent technique can also be used. In countercurrent separations, conditions are used such that there is electrophoretic migration of the protein chiral selector (a chimeric cannulae monomer or polymer of the invention) away from the detector while the analyte migrates past the detector, see, e.g., Chankvetadze (2001) supra.

In alternative aspects, monomer or polymers or mixtures thereof are used to practice the methods of the invention. Chimeric cannulae monomers can have the ability to self-assemble into nanotubules. In one aspect, the chiral resolving power of different polymers (e.g., heteropolymers comprising chimeric and wild type cannulae proteins) and monomers relative to the resolving power of other polymers and monomers can be determined by routine screening, e.g., as described herein. By assembling into a nanotubule, a chimeric cannulae protein becomes a macromolecular structure that possesses distinct microenvironments, including an interior surface, cavity and an exterior surface. In addition, the regular assembly of the subunits into a helical structure introduces additional chirality into the polymer. The polymers of the invention include varying amounts of chirality, as varying amounts of chirality can enhance the enantioselectivity of the composition. The monomers and polymers of the invention can be designed to have varying constrained quaternary (4°) structures. In one aspect, varying constrained quaternary (4°) structures results in varying amounts of chiral selectivity.

In one aspect, the chiral selection is performed under cooling conditions and in the absence of sufficient divalent cation (less than 1 mM) so a cannulae monomer (e.g., a CanA monomer) will not self-assemble during chromatography.

In one aspect, the performance of the chiral selective compositions of the invention are compared to the performance of commercially available chiral selectors. In one aspect, beta-blocker resolutions are performed with capillaries packed with cellobiohydrolase or α₁-acid glycoprotein (ChromTech AB Cheshire, UK) using, e.g., the separation conditions provided by the supplier. Comparisons also can be made to separations obtained using highly sulfated cyclodextrans (Beckman Coulter, Fullerton, Calif.) according to, e.g., methods available from their applications guide. Other characteristics, such as good stability or minimal interference with analyte detection, can also be evaluated. Chimeric cannulae proteins of the invention, including the chimeric CanA polypeptide made by inserting peptide domains into a nonessential surface-exposed domain of CanA (see FIG. 1), can be evaluated using these routine screening methods. FIG. 1 is an illustration of a transmission electron micrograph of nanotubules assembled from recombinant CanA expressed in E. coli.

Because of the macromolecular similarity of nanotubular cannulae polymers to eukaryotic microtubules, any of the analytical methods that have been established in the microtubule field can be used to analyze chimeric cannulae polymers of the invention; see, e.g., Frederiksen, D. W. and L. W. Cunningham. Structural and Contractile Proteins, Part B: The Contractile Apparatus and the Cytoskeleton. 1982. Methods in Enzymology 85[Part B]. In evaluating the chiral selectivity of a chimeric cannulae monomer and/or polymer of the invention and the yield of the chiral selection methods of the invention, light and electron microscopy (e.g., transmission electron microscopes), differential centrifugation, size exclusion chromatography and/or turbidity measurement methods can be used. Each of these methods provides slightly different information about the stability and integrity of the assembled chimeric cannulae polymer.

The assembly and disassembly of polymers of the invention can be followed by measuring changes in solution turbidity, e.g., as described in Purich, D. L., et al. (1982) Microtubule disassembly: a quantitative kinetic approach for defining endwise linear depolymerization. Methods in Enzymology 85[Part B], 439-450. In one aspect, kinetic turbidity measurements are used. Kinetic turbidity measurements can be used to reflect changes in polymer weight concentration. These measurements can be used to determine rates of depolymerization. In one aspect, solution turbidity is monitored spectro-photometrically at 350 nm in a long path length cuvette. The long path length can provide an enhancement of the absorbance change improving sensitivity of the assay. In one aspect, the method comprises a long path length and a temperature-controlled cuvette containing buffers that can range in pH from 3 to 10. Stability of polymer can be measured by diluting concentrated solutions of polymer into the cuvette containing temperature-equilibrated buffer.

In one aspect, the chiral selectivity of chimeric cannulae monomer and/or polymers of the invention and the yield of the chiral selection methods of the invention are determined using a chiller-cooled system. The stability of polymer can be evaluated over a range of temperatures, e.g., from about 4° C. to 80° C. for each buffer pH. In one aspect, if it is not possible to measure accurate depolymerization rates using the rapid dilution method (due to over-dilution of the polymer into the test buffer), a resuspension method can be utilized. In the resuspension method, a wide-bore pipette can be used to resuspend polymer pellets in temperature-equilibrated buffer. The resuspended pellet then can be transferred to a cuvette for analysis. The advantage of this method is the ability to use more concentrated polymer solutions. The drawback, however, is variability introduced by potential shearing of the polymer during resuspension.

In one aspect, the chiral selectivity of chimeric cannulae monomer and/or polymers of the invention and the yield of the chiral selection methods of the invention are determined using differential centrifugation. Differential centrifugation can be used to assess the distribution of monomer protein incorporated into polymer vs. monomer free in solution. The differential centrifugation assay is useful for longer time course stability evaluations. In these assays, polymer that has been assembled under standard conditions at neutral pH can be pelleted by centrifugation and then resuspended in a buffer (e.g., at varied pH, such as from pH 3 to pH 10) and pre-equilibrated at a specified temperature (e.g., at varied temperature, such as a range from about 4° C. to 80° C.). The samples can be incubated at temperature for 2 to 24 hrs and then re-centrifuged to pellet the intact polymer. The supernatant and pellet fractions can be analyzed by SDS-PAGE. The supernatant will contain any soluble monomer (released by polymer depolymerization) and the pellet will contain intact polymer.

In one aspect, the chiral selectivity of chimeric cannulae monomer and/or polymers of the invention and the yield of the chiral selection methods of the invention are determined using size exclusion chromatography. Size exclusion chromatography can be used to analyze the overall size distribution of polymers. Polymer samples can be resuspended in buffer (e.g., at varied pH, such as from pH 3 to pH 10) and incubated for 24 hours at 4° C. Following incubation, the samples can be fractionated, e.g., on a Sephacryl S-1000 column (Amersham Pharmacia, Piscataway, N.J.). This size exclusion column will separate the micron length polymer from shorter polymers and oligomers. Because polymers can be extremely stable at 4° C. and neutral pH, and this buffer treatment can be used as the control.

In one aspect, the chiral selectivity of chimeric cannulae monomer and/or polymers of the invention and the yield of the chiral selection methods of the invention are determined using light microscopy, e.g., video-enhanced light microscopy, including both phase and differential interference contract (DIC) optics. Light microscopy can be used to evaluate the gross morphology of polymers following extended incubations (e.g., between about 24 to 48 hours) at varied pH, such as from pH 3 to pH 10. Light microscopy can provide useful information about the extent of nanotubule polymer bundling. It also can be used to detect the presence of larger protein aggregates.

In one aspect, the chiral selectivity of chimeric cannulae monomer and/or polymers of the invention and the yield of the chiral selection methods of the invention are determined using electron microscopy (EM), e.g., standard negative stain transmission electron microscopy. Electron microscopy can be used to look at the fine structure of nanotubules. EM can be useful for the analysis of periodicity and helicity of the intact polymers. In addition, EM can detect other protein assemblies that may form during incubation at various pH values or in the presence of organic modifiers. Depending on the incubation conditions, eukaryotic microtubules have been shown to assemble into a number of macromolecular structures including ring, sheets, and ribbons, as described, e.g., in Hyams, J. S. and C. W. Lloyd. Microtubules. 1993. New York, Wiley-Liss. The polymers of the invention can be modified to assemble or reassemble into such alternate structures.

Chimeric cannulae protein of the invention can be abundantly and economically expressed as a recombinant protein, as discussed below.

Cannulae Polypeptide and Peptides

The invention provides chimeric polypeptides comprising at least a first domain comprising a cannulae polypeptide and at least a second domain comprising a heterologous polypeptide or peptide. The chimeric (fusion) cannulae polypeptides of the invention can be recombinant proteins encoded by nucleic acids comprising fusion of the sequence of a cannulae monomer to other protein or peptide coding sequences (heterologous sequences) to produce cannulae fusion (chimeric) proteins. However, the chimeric (fusion) cannulae polypeptides of the invention can be joined to the heterologous polypeptide or peptide by any means, including linkers. The chimeric (fusion) cannulae polypeptides of the invention can be partly or entirely synthetic. In one aspect, the chimeric monomers of the invention can form dimers, trimers (polymers of any length) and/or they can assemble, e.g., self-assemble, into a higher order structure, e.g., a quaternary structure, such as a nanotubule. The heterologous sequences can be added to the cannulae protein's amino- or carboxy-terminal end, or, they can be added internal to the cannulae protein.

In one aspect, a subsequence of a chimeric (fusion) cannulae polypeptide, or, a cannulae protein used in a composition of the invention (e.g., a drug delivery vehicle), of the invention is removed. In one aspect, a subsequence of a chimeric (fusion) cannulae polypeptide of the invention, or, a cannulae protein used in a composition of the invention, is removed and replaced by a heterologous polypeptide or peptide. Alternatively, the heterologous polypeptide or peptide can be added to another section of the monomer (i.e., distal to the removed subsequence). The removed subsequence can be amino- or carboxy-terminal, or, it can be internal to the cannulae protein. In one aspect, the subsequence of fusion (hybrid) CanA protein that is removed and replaced by a heterologous polypeptide or peptide is a 14 residue motif consisting of residue 123 to residue 136 of SEQ ID NO:2 (i.e., “PDKTGYTNTSIWVP”), or, a 17 residue motif located at amino acid residue 123 to residue 139 of SEQ ID NO:2, (i.e., “PDKTGYTNTSIWVPGEP”). When the CanA monomer is in polymeric nanotubular form, a 14 residue motif consisting of residue 123 to residue 136 of SEQ ID NO:2 or a 17 residue motif located at amino acid residue 123 to residue 139 of SEQ ID NO:2 is expressed on the outer surface of the nanotubule. In one aspect, the tubule can then act as a high-density chiral selector. The surface-exposed 14 or 17 amino acid domain in CanA is not essential for self-assembly of nanotubules. Thus, these domains can serve as a site for the insertion of peptides, e.g., with chiral selector properties, ligand binding properties, and the like.

Once assembled, e.g., as a nanotubule, chimeric cannulae proteins of the invention, or, a cannulae protein used in a composition of the invention, can serve as a molecular scaffold that displays its heterologous sequence (its chimeric/fusion protein partner) in a defined orientation in a regular, helical array. This functional flexibility offers the opportunity to display a large variety of recombinant proteins on the surface of a nanotubule to create chiral selectors with a wide range of applications. The heterologous sequences can be chiral selection motifs, enzymes, active sites, epitopes, ligands, receptors, antigens, antibodies or antigen binding sites, nucleic acid binding proteins, and the like.

In one aspect, the chimeric cannulae monomers are overexpressed in a host cell, e.g., a bacteria such as an E. coli. In one aspect, the overexpressed polypeptide is modified by nucleic acid mutagenesis and/or directed protein evolution, as described herein.

The cannulae domain(s) of the chimeric polypeptides of the invention, or polymers used in compositions of the invention (e.g., drug delivery vehicles), can comprise a CanA polypeptide as set forth in SEQ ID NO:2 (encoded by SEQ ID NO:1); a CanB polypeptide as set forth in SEQ ID NO:4 (encoded by SEQ ID NO:3); a CanC polypeptide as set forth in SEQ ID NO:6 (encoded by SEQ ID NO:5); a CanD polypeptide as set forth in SEQ ID NO:8 (encoded by SEQ ID NO:7); a CanE polypeptide as set forth in SEQ ID NO:10 (encoded by SEQ ID NO:9), or a combination thereof. The cannulae domain of the chimeric polypeptides of the invention, or polymers used in compositions of the invention (e.g., drug delivery vehicles), also can comprise a polypeptide having a 50%, 55%, 60%, 65%, 70%, 75%, 80%, 85%, 90%, 95% or 98% sequence identity to polypeptide as set forth in SEQ ID NO:2, SEQ ID NO:4, SEQ ID NO:6, SEQ ID NO:8, SEQ ID NO:10, wherein the cannulae domain polypeptide can form a nanotubule and/or can act as a chiral selector (in monomeric or polymeric form). The cannulae domain of the chimeric polypeptides of the invention also can comprise a polypeptide encoded by a nucleic acid having a 50%, 55%, 60%, 65%, 70%, 75%, 80%, 85%, 90%, 95% or 98% sequence identity to a nucleic acid as set forth in SEQ ID NO:1, SEQ ID NO:3, SEQ ID NO:5, SEQ ID NO:7, SEQ ID NO:9, wherein the cannulae domain polypeptide can form a nanotubule and/or can act as a chiral selector (in monomeric or polymeric form). The cannulae domains of the chimeric polypeptides of the invention, or polymers used in compositions of the invention (e.g., drug delivery vehicles), can comprise two or more of these proteins, including mixtures of CanA, CanB, CanC, CanD and/or CanE.

The term protein or polypeptide sequence or amino acid sequence includes an oligopeptide, peptide, polypeptide, or protein sequence, or to a fragment, portion, or subunit of any of these, and to naturally occurring or synthetic molecules. The terms “polypeptide” and “protein” include amino acids joined to each other by peptide bonds or modified peptide bonds, i.e., peptide isosteres, and may contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms.

The invention also comprises “variants” of the chimeric polynucleotides or polypeptides of the invention, and methods of making them, wherein the variant polynucleotides are modified at one or more base pairs, codons, introns, exons, or the variant polypeptides are modified at one or more amino acid residues (respectively) yet still encode enzymes that retain the activity or have a modified activity of a chimeric polypeptide of the invention. Variants can be produced by any number of means included methods such as, for example, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, GSSM™ and any combination thereof. Techniques for producing variant chimeric polypeptides having activity at a pH or temperature, for example, that is different from a template chimeric polypeptide, are included herein. The term “saturation mutagenesis” or Gene Site Saturation Mutagenesis™ or “GSSM™” includes a method that uses degenerate oligonucleotide primers to introduce point mutations into a polynucleotide, as described in detail, below. The term “optimized directed evolution system” or “optimized directed evolution” includes a method for reassembling fragments of related nucleic acid sequences, e.g., related genes, and explained in detail, below. The term “synthetic ligation reassembly” or “SLR” includes a method of ligating oligonucleotide fragments in a non-stochastic fashion, and explained in detail, below.

The phrase “substantially identical” in the context of two nucleic acid sequences or polypeptides used in the compositions or methods of the invention refers to two or more sequences that have at least 50 nucleotide or amino acid residue identity over a region of at least about 100 residues, when compared and aligned for maximum correspondence, as measured using one of the known sequence comparison algorithms or by visual inspection. Substantially identical nucleic acid sequences may have at least 55%, 60%, 65%, 70%, 75%, 80%, 85%, 90% or 95% nucleotide or amino acid residue identity and this identified may also extend over at least about 150-200 residues, over the entire length of the coding regions of the nucleic acid sequences or polypeptides, or over the entire length of the nucleic acid sequences or polypeptides, e.g., the exemplary SEQ ID NO:2, SEQ ID NO:4, SEQ ID NO:6, SEQ ID NO:8 or SEQ ID NO:10, used in the drug delivery systems or chimeric polypeptide of the invention. In one aspect, “substantially identical” in the context of a first nucleic acid sequence (e.g., e.g., the exemplary SEQ ID NO:1, SEQ ID NO:3, SEQ ID NO:5, SEQ ID NO:7 or SEQ ID NO:9, or encoding the exemplary SEQ ID NO:2, SEQ ID NO:4, SEQ ID NO:6, SEQ ID NO:8 or SEQ ID NO:10) and a second nucleic acid sequence refers to the first and second sequences having at least 50% nucleotide residue identity over at least about 100 residues, when compared and aligned for maximum correspondence, as measured using one of the known sequence comparison algorithms or by visual inspection. In one aspect, “substantially identical” in the context of a first amino acid sequence (e.g., the exemplary SEQ ID NO:2, SEQ ID NO:4, SEQ ID NO:6, SEQ ID NO:8 or SEQ ID NO:10) and a second amino acid sequence refers to the first and second amino acid sequences having at least 50% amino acid residue identity over at least about 100 residues, when compared and aligned for maximum correspondence, as measured using one of the known sequence comparison algorithms or by visual inspection.

“Fragments” as used herein are a portion of a naturally occurring or recombinant protein, which can exist in at least two different conformations. Fragments can have the same or substantially the same amino acid sequence as the naturally occurring protein. “Substantially the same” means that an amino acid sequence is largely, but not entirely, the same, but retains at least one functional activity of the sequence to which it is related. In general two amino acid sequences are “substantially the same” or “substantially homologous” if they are at least about 70, but more typically about 85% or more identical. Fragments, which have different three-dimensional structures than the naturally occurring protein, are also included. An example of this is a “pro-form” molecule, such as a low activity proenzyme that can be modified by cleavage to produce a mature enzyme with significantly higher activity.

Additionally a “substantially identical” amino acid sequence is a sequence that differs from a reference sequence (e.g., the exemplary SEQ ID NO:2, SEQ ID NO:4, SEQ ID NO:6, SEQ ID NO:8 or SEQ ID NO:10) by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from a haloalkane dehalogenase polypeptide, resulting in modification of the structure of the polypeptide, without significantly altering its biological activity or properties. For example, amino- or carboxyl-terminal amino acids that are not required for haloalkane dehalogenase biological activity can be removed. Modified polypeptide sequences of the invention can be assayed for haloalkane dehalogenase biological activity by any number of methods, including contacting the modified polypeptide sequence with an haloalkane dehalogenase substrate and determining whether the modified polypeptide decreases the amount of specific substrate in the assay or increases the byproducts of the enzymatic reaction of a functional haloalkane dehalogenase polypeptide with the substrate.

In one aspect, nucleic acids encoding the chimeric polypeptides or and drug delivery systems of the invention are cloned and over-expressed in a host cell, e.g., E. coli. Purified recombinant chimeric cannulae protein can self-assemble into nanotubules. The presence of a divalent cation may be needed, depending on the conditions and mixture of polypeptides comprising the nanotubular assembly or the presence of proteins that catalyze or facilitate tubule assembly. The divalent cation may be Ca²⁺, Mg²⁺, Cu²⁺, Zn²⁺, Sr²⁺, Ni²⁺, Mn²⁺ and/or Fe²⁺. In one aspect, a single divalent cation is needed, e.g., Ca²⁺ or Mg²⁺. In another aspect, both Ca²⁺ and Mg²⁺ are needed for chimeric cannulae protein can self-assemble into nanotubules. In one aspect, the divalent cation(s) are present in millimolar concentrations.

The chimeric polypeptides and drug delivery systems of the invention can comprise the cannulae polypeptides CanA, CanB, CanC, CanD and/or CanE (including SEQ ID NO:2, SEQ ID NO:4, SEQ ID NO:6, SEQ ID NO:8 or SEQ ID NO:10, and sequences substantially identical thereto), and subsequences and mixtures thereof. In the following alignment, CanA and CanA_pep stand for nucleic acid SEQ ID NO:1 and its corresponding amino acid SEQ ID NO:2, respectively; CanB and CanB_pep stand for nucleic acid SEQ ID NO:3 and its corresponding amino acid SEQ ID NO:4, respectively; CanC and CanC_pep stand for nucleic acid SEQ ID NO:5 and its corresponding amino acid SEQ ID NO:6, respectively; CanD_partial stands for nucleic acid SEQ ID NO:7 or its corresponding amino acid SEQ ID NO:8; and CanE_partial stands for nucleic acid SEQ ID NO:9 or its corresponding amino acid SEQ ID NO:10.

Nucleic acid alignment for SEQ ID NOS:1, 3, 5, 7, and 9:

| | | |
|---|---|---|
| 1 | | 50 |
| CanA | (1) | GTGAAGTACACAACCCTAGCTATAGCGGGTATTATTGCCTCGGCTGCCGC |
| CanB | (1) | GTGAAGCCTACGGCTCTAGCCCTGGCTGGTATCATTGCCTCGGCTGCCGA |
| CanC | (1) | ATGAGGTACACGACCCTAGCTCTGGCCGGCATAGTGGCCTCGGCTGCCGC |
| CanD_partial | (1) | —————————————————————————————————————————————————— |
| CanE_partial | (1) | —————————————————————————————————————————————————— |
| Consensus | (1) | TGA G AC C CTAGC T GC GG AT T GCCTCGGCTGCCG |
| | | 51 100 |
| CanA | (51) | CCTCGCCCTCCTAGCAGGCTTCGCCACCACCCAGAGCCCCCTCAACAGCT |
| CanB | (51) | CCTCGCCCTGCTAGCAGGCTTCGCCACCACCCAGAGCCCGCTCAACAGCT |
| CanC | (51) | CCTCGCCCTGCTAGCAGGCTTCGCCACGACCCAGAGCCCGCTAAGCAGCT |
| CanD_partial | (1) | ——————————————————————————————————————————————AGCT |
| CanE_partial | (1) | ——————————————————————————————————————————————AGCT |
| Consensus | (51) | CCTCGCCCT CTAGCAGGCTTCGCCAC ACCCAGAGCCC CT A CAGCT |
| | | 101 150 |
| CanA | (101) | TCTACGCCACCGGTACAGCACAGGCAGTAAGCGAGCCAATAGACGTAGAA |
| CanB | (101) | TCTACGCCACCGGCACAGCAGCCGCAACAAGCGAGCCAATAGACGTAGAG |
| CanC | (101) | TCTACGCCACCGGCACAGCACAAGCAGTAAGCGAGCCAATAGACGTAGAG |
| CanD_partial | (5) | TCTACGCCACCGGCACAGCACAGGCAGTAAGCGAGCCAATAGACGTGGTA |
| CanE_partial | (5) | TCTACGCCACCGGCACAGCAGAGGCAACAAGCGAGCCAATAGACGTTGTA |
| Consensus | (101) | TCTACGCCACCGGCACAGCACAGGCAGTAAGCGAGCCAATAGACGTAGAA |
| | | 151 200 |
| CanA | (151) | AGCCACCT———CGGCAGCATAACCCCCGCAGCCGGCGCACAGGGCAGTGA |
| CanB | (151) | AGCCACCT———CAGCAGCATAGCCCCTGCTGCTGGCGCACAGGGCAGCCA |
| CanC | (151) | AGCCACCTAGACAACACCATAGCCCCTGCTGCCGGTGCACAGGGCTACAA |
| CanD_partial | (55) | AGCAGCCTCGGTACG———CTAAATACTGCCGCTGGTGCACAGGGTAAGCA |
| CanE_partial | (55) | AGCAACCTTAACACGGCCATAGCCCCTGCTGCCGGCGCCCAGGGCAGCGT |
| Consensus | (151) | AGCCACCT CA CA CATAGCCCCTGCTGCCGGCGCACAGGGCAGC A |
| | | 201 250 |
| CanA | (198) | CGACATAGGTTACGCAATAGTGTGGATAAAGGACCAGGTCAATGATGTAA |
| CanB | (198) | GGACATAGGCTACTTCAACGTGACCGCCAAGGATCAAGTGAACGTGACAA |
| CanC | (201) | GGACATGGGCTACATTAAGATAACTAACCAGTCAAAAGTTAATGTAATAA |
| CanD_partial | (102) | GACGCTAGGAGACATAACAATATATGCGCACAATGACGTGAACATAACAA |
| CanE_partial | (105) | GGGCATAGGCAGCATAACAATAGAGAACAAGACTGACGTGAACGTTGTGA |
| Consensus | (201) | GGACATAGGCTACATAA AATA A CAAG AT A GTGAACGT ATAA |
| | | 251 300 |
| CanA | (248) | AGCTGAAGGTGACCCTGCGTAACGCTGAGCAGCTAAAGCCCTACTTCAAG |
| CanB | (248) | AGATAAAGGTGACCCTGGCTAACGCTGAGCAGCTAAAGCCCTACTTCAAG |
| CanC | (251) | AGCTGAAGGTGACTCTCGCTAACGCCGAGCAGCTAAAGCCCTACTTCGAC |
| CanD_partial | (152) | AGCTAAAGGTCACGCTTGCTAACGCTGCACAGCTAAGACCATACTTCAAG |
| CanE_partial | (155) | AGCTGAAGATAACCCTCGCCAACGCTGAGCAGCTAAAGCCCTACTTCGAC |
| Consensus | (251) | AGCTGAAGGTGACCCT GCTAACGCTGAGCAGCTAAAGCCCTACTTCAAG |
| | | 301 350 |
| CanA | (298) | TACCTACAGATACAGATAACAAGCGGCTATGAGACGAACAGCACAGCTCT |
| CanB | (298) | TACCTACAGATAGTGCTAAAGAGCG————————————————————————— |
| CanC | (301) | TACCTACAGCTAGTACTCACAAGCAAC——————————————————GCCAC |
| CanD_partial | (202) | TACCTGATAATAAAGCTAGTAAGCCT——————————————GGACAGC—AA |
| CanE_partial | (205) | TACCTACAGATAGTGCTAAAGAGCGT——————————————TGACAGC—AA |
| Consensus | (301) | TACCTACAGATAGTGCTAA AAGCG ACAGC A |
| | | 351 400 |
| CanA | (348) | AGGCAACTTCAGCGAGACCAAGGCTGTGATAAGCCTCGACAACCCCAGCG |
| CanB | (323) | AGGTAGCTGA——CGAGATCAAGGCCGTAATAAGCATAGACAAGCCTAGCG |
| CanC | (333) | TGGCACCGACA———TGGTTAAGGCTGTGCTAAGCCTCGAGAAGCCTAGCG |
| CanD_partial | (237) | CGGCAACGAGTCCGAGGAAAAGGGCATGATAACTCTATGGAAGCCTTACG |
| CanE_partial | (240) | CGAGATCAAGGCTG————————————TGCTAAGCCTCGAGAAGCCCAGCG |
| Consensus | (351) | GGCA C A CGAG AAGGC GTGATAAGCCTCGAGAAGCCTAGCG |
| | | 401 450 |
| CanA | (398) | CCGTGATAGTACTAGACAAGGAGGATATAGCAGTGCTCTATCCGGACAAG |
| CanB | (371) | CCGTCATAATACTAGACAGCCAGGA————————————————————————— |
| CanC | (380) | CAGTCATAATACTAGACAACGATGA————————————————————————— |
| CanD_partial | (287) | CCGTGATAATACTAGACCATGAAGA————————————————————————— |
| CanE_partial | (278) | CAGTCATAATACTGGACAACGAGGA————————————————————————— |
| Consensus | (401) | CCGTCATAATACTAGACAACGAGGA |
| | | 451 500 |
| CanA | (448) | ACCGGTTACACAAACACTTCGATATGGGTACCCGGTGAACCTGACAAGAT |
| CanB | (396) | ————————————————CTTCGACA——————————————G——————————— |
| CanC | (405) | ————————————————CTACGATA——————————————G——————————— |
| canD_partial | (312) | ————————————————TTTCAACAACGACA———————————————————— |
| canE_partial | (303) | ————————————————CTTCCAGGGCGGC————————————————————— |
| Consensus | (451) | CTTCGA A G |
| | | 501 550 |
| CanA | (498) | AATTGTCTACAACGAGACAAAGCCAGTAGCTATACTGAACTTCAAGGCCT |
| canB | (405) | ——————CAACAACAGAGCAAAG——ATAAGCGCCACTG——————————CCT |
| CanC | (414) | ——————CACTAACAAGATACAGCTA—AAGGTAGA———A———————G—CCT |
| canD_partial | (326) | —————TCGACAATGACGGCAACAATGACGCCAAGATAAGGGTTGTAGCCT |
| canE_partial | (316) | ———————GACAACCAGTGCCAGATAGACGCCACC————————————GCCT |
| Consensus | (501) | C ACAAC AG AAAG AGAAGC A A T A GCCT |
| | | 551 600 |
| CanA | (548) | TCTACGAGGCTAAGGAGGGTATGCTATTCGACAGCCTGCCAGTGATATTC |
| canB | (437) | ACTACGAGGCTAAGGAGGGCATGCTATTCGACAGCCTACCGCTAATATTC |
| CanC | (446) | ACTATGAGGCTAAGGAGGGCATGCTATTCGACAGCCTACCAGTAATACTG |
| canD_partial | (371) | ACTATGAGGCTAAGGAGGGTATGCT————————————————————————— |
| canE_partial | (347) | ACTACGAGGCTAAGGAGGGTATGCTA———————————————————————— |
| Consensus | (551) | ACTACGAGGCTAAGGAGGGTATGCTATTCGACAGCCT CC T ATA T |
| | | 601 642 |
| CanA | (598) | AACTTCCAGGTGCTACAAGTAGGCTAA——————————————— |
| canB | (487) | AACATACAGGTGCTAAGCGTCAGCTAA——————————————— |
| CanC | (496) | AACTTCCAGGTACTGAGCGCCGCTTGCAGTCCCTTGTGGTGA |
| canD_partial | (396) | —————————————————————————————————————————————————— |
| canE_partial | (373) | —————————————————————————————————————————————————— |
| Consensus | (601) | AAC T CAGGT CT G T |

Amino Acid Alignment for SEQ ID NOS:2, 4, 6, 8, and 10:

| | | |
|---|---|---|
| 1 | | 50 |
| CanA_pep | (1) | VKYTTLAIAGIIASAAALALLAGFATTQSPLNSFYATGTAQAVSEPIDVE |
| CanB_pep | (1) | VKPTALALAGIIASAADLALLAGFATTQSPLNSFYATGTAAATSEPIDVE |
| CanC_pep | (1) | MRYTTLALAGIVASAAALALLAGFATTQSPLSSFYATGTAQAVSEPIDVE |
| CanD_partial | (1) | ————————————————————————————————SFYATGTAQAVSEPIDVV |
| CanE_partial | (1) | ————————————————————————————————SFYATGTAEATSEPIDVV |
| Consensus | (1) | VK T LALAGIIASAA LALLAGFATTQSPL SFYATGTAQAVSEPIDVE |
| | | 51 100 |
| CanA_pep | (51) | SHLG—SITPAAGAQGSDDIGYAIVWIKDQVNDVKLKVTLRNAEQLKPYFK |
| CanB_pep | (51) | SHLS—SIAPAAGAQGSQDIGYFNVTAKDQVNVTKIKVTLANAEQLKPYFK |
| CanC_pep | (51) | SHLDNTIAPAAGAQGYKDMGYIKITNQSKVNVIKLKVTLANAEQLKPYFD |
| CanD_partial | (19) | SSLGTLNT—AAGAQGKQTLGDITIYAHNDVNITKLKVTLANAAQLRPYFK |
| CanE_partial | (19) | SNLNTAIAPAAGAQGSVGIGSITIENKTDVNVVKLKITLANAEQLKPYFD |
| Consensus | (51) | SHL SIAPAAGAQGS DIGYI I K VNVVKLKVTLANAEQLKPYFK |
| | | 101 150 |
| CanA_pep | (100) | YLQIQITSGYETNSTALGNFSETKAVISLDNPSAVIVLDKEDIAVLYPDK |
| CanB_pep | (100) | YLQIVLKSEVAD—————————EIKAVISIDKFSAVIILDSQDFDSNNR—— |
| CanC_pep | (101) | YLQLVLTSNATG———————TDMVKAVLSLEKPSAVIILDNDDYDSTN——— |
| CanD_partial | (68) | YLIIKLVSLDSNG—————NESEEKGMITLWKPYAVIILDHEDFNNDID—— |
| CanE_partial | (69) | YLQIVLKSVDSN—————————EIKAVLSLEKPSAVIILDNEDFQG————— |
| Consensus | (101) | YLQIVL S S EIKAVISLDKPSAVIILD EDF |
| | | 151 200 |
| CanA_pep | (150) | TGYTNTSIWVPGEPDKIIVYNETKPVAILNFKAFYEAKEGMLFDSLPVIF |
| CanB_pep | (139) | ——————————————————————————AKISATAYYEAKEGMLFDSLPLIF |
| CanC_pep | (141) | ————————————————KIQ—————————LKVEAYYEAKEGMLFDSLPVIL |
| CanD_partial | (111) | ——————————————————N——DGNNDAKIRVVAYYEAKEGM————————— |
| CanE_partial | (105) | ——————————————————————GDNQCQIDATAYYEAKEGML———————— |
| Consensus | (151) | A I AYYEAKEGMLFDSLPVI |
| | | 201 214 |
| CanA_pep | (200) | NFQVLQVG—————— |
| CanB_pep | (163) | NIQVLSVS—————— |
| CanC_pep | (166) | NFQVLSAACSPLW— |
| CanD_partial | (132) | —————————————— |
| CanE_partial | (125) | —————————————— |
| Consensus | (201) | N QVL |

The polymer may have a shape of a short fiber, and therefore is also called “polymer fiber.” The polymer fiber is made from monomeric protein units, e.g. Can A: 182 amino acids: MW=19,830 Daltons, having a sequence of SED ID NO:2. The secondary structure of the protein may be mainly β-sheets.

The protein subunits in the polymer are arranged in a right-handed or left-handed, two-stranded helix. Occasionally, the polymer fibers made up of a three-handed helix may be observed. The periodicity (the distance of one helix turn to the next) of the polymer is 4.4 nm. The polymer has a unique quaternary structure. The polymer fiber has an outer diameter of 25 nm and inner diameter, 21 nm (in suspension). Under an electronic microscope, the dry negatively stained polymer fibers exhibit an outer diameter of 32 nm due to collapsing. Length of the polymer fiber is mostly between 3 and 5 micrometers. Some of the polymer fibers may reach a length from 10 to 25 micrometers. The polymer fibers may form bundles of tens and hundreds of fibers with an overall diameter of 100 to 500 nm. Occasionally the bundle may reach an overall diameter of 4,000 nm. The polymer fiber is at least stable up to 128° C.

CanA nanotubules can exhibit remarkable heat stability, e.g. temperatures to 128° C. and stability in 2% SDS at 100° C. for at least 60 minutes. Purified recombinant CanB protein will also form nanotubular structures but they are less regular and not as heat stable as the nanotubules assembled from CanA. Purified, recombinant CanC does not self-assemble into nanotubules. Together, CanA (SEQ ID NO:2), CanB (SEQ ID NO:4), and CanC (SEQ ID NO:6) represent three very similar proteins that exhibit significantly different polymerization potentials in vitro.

TABLE 1
Comparison of amino acid sequences of CanA, CanB, CanC.

| | | | |
|---|---|---|---|
| Protein | CanA | CanB | CanC |

| | | | | | |
|---|---|---|---|---|---|
| CanA | 100% | | | | |
| CanB | 60% | Identical | 100% | | |
| | 64% | Similar | | | |
| CanC | 55% | Identical | 68% | Identical | 100% |
| | 62% | Similar | 77% | Similar | |

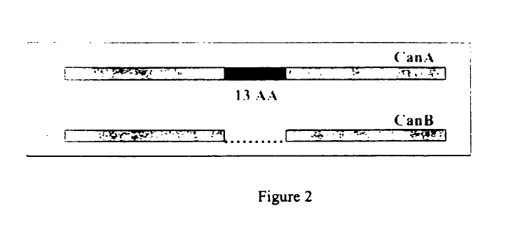

One difference between CanA and CanB is the 14 amino acid insertion near the middle of the CanA sequence (see FIG. 2). Immunoelectron microscopy and an antibody specific for this 14 amino acid sequence have been used to determine that this sequence is displayed on the surface of the assembled nanotubule. The absence of this corresponding sequence in CanB demonstrates that this peptide domain is nonessential for nanotubule assembly. Therefore, it is possible to remove this sequence and replace it with a peptide domain that alters the structure of CanA. In one aspect, replacing the endogenous 14 residue motif with a heterologous peptide changes the enantioselectivity of CanA.

Recombinant chimeric proteins of the invention, and polypeptides used in the drug delivery systems of the invention can be expressed in a cell, e.g., a bacteria, such as E. coli, and purified away from host proteins by using heat treatment to denature and precipitate (e.g., E. coli) protein. The soluble heat stable protein (e.g., CanA) can be recovered from the supernatant following centrifugation. The chimeric protein can be assembly-competent at this stage. In one aspect, the self-assembly reaction is initiated by addition of millimolar concentrations of Ca⁺⁺ and Mg⁺⁺. In one aspect, following assembly of the nanotubules, they are stable in cation-free buffer and buffers containing up to 20 mM chelator, e.g., EDTA, EGTA.

Colloidal Stability. Nanotubules of the invention can interact at different levels by pairing, bundling, entangling (excluded volume interaction) and electrostatic cross-linking (bridging by divalent cations). The different types of aggregates have an increasing dimensionality from a pair of rods to an interconnected network. The bundling of CanA nanotubules appears to be a magnesium-dependent process. In the absence of magnesium, CanA displays minimal bundling. However, upon the addition of millimolar concentrations of magnesium, CanA nanotubules will form bundles visible by standard phase contrast light microscopy.

Nanotubule Stiffness. CanA nanotubules have been imaged under the transmission electron (TEM) and atomic force microscopes (AFM). From analyses of thermal vibrations of a single fiber in vacuum under the TEM, it was found that the CanA bending modulus is about 5±2 GPa. This result is somewhat greater than other rigid biopolymers of the same dimensions, such as microtubules which have a bending modulus of nearly 2 GPa, and comparable to the bending moduli of the strongest synthetic polymer fibers like Poly(6-amide) or Poly(methylmethacrylate), or, PLEXIGLAS™).

In one aspect, the chimeric CanA proteins of the invention are used as chiral selectors, e.g., in capillary electrophoresis. Serum albumin was one of the first proteins used as a chiral stationary phase for the successful separation of enantiomers, see, e.g., (reviewed in Allenmark, 1998). Numerous proteins have been used to accomplish many enantioseparations using capillary electrophoresis methods. These proteins include α₁-acid glycoprotein, avidin, ovomucoid, transferrin, cytochrome c, lysozyme, pepsin, cellulase, and cellobiohydrolase see, e.g., Tanada (2001) supra. Proteins are favorable for use as chiral selectors because they frequently can be used for a wide variety of enantioseparations, see, e.g., Lloyd (1995) J. of Chromatography A. 694:285-296. In addition, because proteins can be used for chiral separations in aqueous buffers, they are a good choice for the analysis of samples derived from biological material, see, e.g., Busch (1993) supra. Accordingly, in alternative aspects, the chimeric CanA polypeptides of the invention comprise chiral selection motifs from serum albumin, α₁-acid glycoprotein, avidin, ovomucoid, transferrin, cytochrome c, lysozyme, pepsin, cellulase and cellobiohydrolase. The chimeric CanA of the invention can comprise any peptide motif having a chiral selection capability. These motifs can be inserted into a CanA or added to a CanA. In one aspect, they are used to replace a subsequence of CanA that has been removed, e.g., a 14 residue motif consisting of residue 123 to residue 136 of SEQ ID NO:2 or a 17 residue motif located at amino acid residue 123 to residue 139 of SEQ ID NO:2.

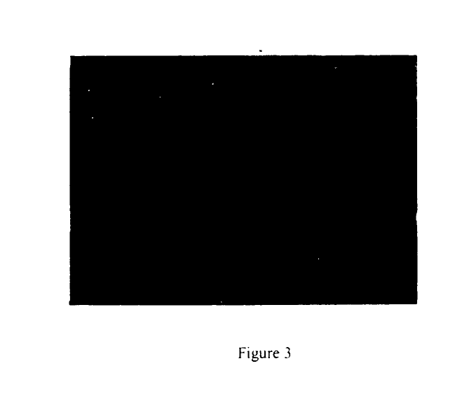

A chimeric monomer or polymer of the invention can comprise a detectable moiety. In one aspect, the heterologous motif is a detectable moiety, e.g., a green fluorescent protein. In one aspect, the invention provides a nanotubule comprising chimeric monomers comprising green fluorescent protein motifs. These monomers and nanotubules can be used to study nanotubule formation, dissolution and function. For example, FIG. 3 is an illustration of an immunofluorescent light microscope image of nanotubules assembled from a fusion protein generated by fusing the CanA open reading frame (SEQ ID NO:1) to the open reading frame of the green fluorescent protein ZSGREEN™ (BD Biosciences Clontech, Palo Alto, Calif.).

The invention provides enantioseparation methods using proteins free in solution as buffer additives, as described, e.g., in Busch (1993) supra, and using proteins immobilized by a variety of methods, as described, e.g., in Tanaka (2001) supra; Ito (2001) J. of Chromatography A 925:41-47. There are advantages and disadvantages to both approaches. By using proteins in solution, the native conformation of the protein is maintained resulting in a more uniform presentation of the sites involved in generating chiral resolution. However, in capillary electrophoresis-based methods, the presence of protein in the buffer solutions can produce extremely high background UV absorption. This limitation has been addressed by using partial filling and countercurrent techniques that allow relatively high concentration protein solutions to be used without causing background problems at the detector. Partial filling and countercurrent techniques are well known in the art, as, e.g., described in Tanaka (2001) supra; Chankvetadze (2001) supra.

In contrast, the use of immobilization techniques allows for the production of capillaries with high concentrations of the protein chiral selector. The potential drawback to these approaches is the heterogeneity introduced by the method of protein immobilization. This heterogeneity is particularly important when analyzing protein-ligand interactions (see, e.g., Lloyd (1995) supra). Changes in protein conformation introduced as a result of the immobilization method can significantly alter protein-ligand interactions and these types of analyses are therefore more often performed using protein chiral selectors in free solution.

Given their capacity for stereospecific molecular recognition (see, e.g., Lakshmi (1997) Nature 388:758-760; Henriksson (1996) supra), enzymes and apoenzymes are a source of chiral selectors used in the compositions and methods of the invention. Thus, the invention provides chimeric monomers and polymers, including nanotubules, comprising chiral selector enzymes and apoenzymes and chiral selector peptide motifs of enzymes and apoenzymes, such as enzyme active site motifs. The chimeric monomers and polymers, including nanotubules, of the invention can comprise any enzymes or apoenzymes, or any enzyme active site motif. For example, the chimeric monomers and polymers, including nanotubules, and active site motifs of the invention can be derived from glycosyltransferases, glycosylhydrolases, nitrilases, esterases, amidases, lipases, polymerases, cellulases, hydrolases, deaminases, nitroreductases and the like.

Polypeptides and peptide for making and/or using the chimeric monomers and polymers of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides for making and/or using the chimeric monomers and polymers of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

The peptides and polypeptides for making and/or using the chimeric monomers and polymers of the invention, and used in the drug delivery systems of the invention, can also be glycosylated. The glycosylation can be added post-translationally either chemically or by cellular biosynthetic mechanisms, wherein the later incorporates the use of known glycosylation motifs, which can be native to the sequence or can be added as a peptide or added in the nucleic acid coding sequence. The glycosylation can be O-linked or N-linked.

The peptides and polypeptides for making and/or using the chimeric monomers and polymers used in compositions of the invention (e.g., drug delivery vehicles, fibers), as defined above, include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, in one aspect, a mimetic composition is within the scope of the invention if it has an amylase activity.

Polypeptide mimetic compositions can contain any combination of non-natural structural components. In alternative aspect, mimetic compositions of the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide of the invention can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A residue, e.g., an amino acid, of a polypeptide for making and/or using the chimeric monomers and polymers of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but also can be referred to as the R- or S-form.

The invention also provides methods for modifying the chimeric polypeptides of the invention by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983).

Solid-phase chemical peptide synthesis methods can also be used to synthesize the polypeptide or fragments for making and/or using the chimeric monomers and polymers of the invention. Such method are known in the art, see, e.g., Merrifield (1963) J. Am. Chem. Soc. 85:2149-2154; Stewart, J. M. and Young, J. D., Solid Phase Peptide Synthesis, 2nd Ed., Pierce Chemical Co., Rockford, Ill., pp. 11-12; and have been employed in commercially available laboratory peptide design and synthesis kits (Cambridge Research Biochemicals). Such commercially available laboratory kits have generally utilized the teachings of H. M. Geysen et al, Proc. Natl. Acad. Sci., USA, 81:3998 (1984) and provide for synthesizing peptides upon the tips of a multitude of “rods” or “pins” all of which are connected to a single plate. When such a system is utilized, a plate of rods or pins is inverted and inserted into a second plate of corresponding wells or reservoirs, which contain solutions for attaching or anchoring an appropriate amino acid to the pin's or rod's tips. By repeating such a process step, i.e., inverting and inserting the rod's and pin's tips into appropriate solutions, amino acids are built into desired peptides. In addition, a number of available FMOC peptide synthesis systems are available.

Drug Delivery Systems

The invention provides drug delivery systems, e.g., nanoscale drug delivery vehicles for targeted drug delivery, and methods for making them. The drug delivery systems of the invention can comprise fibers made from a self-assembled protein polymer, e.g., a cannulae protein, such as CanA, CanB, CanC, CanD or CanE, including, e.g., the exemplary polypeptides SEQ ID NO:2, SEQ ID NO:4, SEQ ID NO:6, SEQ ID NO:8, or SEQ ID NO:10, subsequences thereof, and sequences substantially identical thereto.

The term “nanoscale” refers to a device, a material containing a structure, or other items having a size in the range of nanometers. In one aspect, a device, material, or structure is referred to as “nanoscale” if the device, material, or structure has a dimensional size in the range of about 1 nm to 1000 nm.

The term “nanoscale delivery vehicle” refers to a nanoscale supramolecular structure that is capable of encapsulating at least one molecule, traveling to a particular location in a human or animal body and releasing the molecule at the particular location. There are many examples of nanoscale delivery vehicles such as the hollow rod described in Jelinski, Biologically related aspects of nanoparticles, nanostructured materials, and nanodevices, In Nanostructure Science and Technology, A WTEC Panel Report prepared under the guidance of the Interagency Working Group on Nanoscience, Engineering and Technology (1999). Sometimes, this type of nanoscale delivery vehicle is also referred to as a “nanocapsule,” “nanotube,” “nanoparticle,” “nanocage,” “micelle,” or by other similar names.

The term “polymer” refers to a large molecule that contains a plurality of repeating units or monomers. The linkages between these repeating units or monomers may be covalent bonds, hydrogen bonding, van der Waals force or other non-covalent interactions. The polymer may be formed by self-assembly of the monomers with or without a template molecule. Alternatively, the polymer may be formed by a chain polymerization reaction or a step polymerization reaction. Preferably, “polymer” refers to a molecule having a molecular weight of more than 5,000 Daltons. More preferably, “polymer” refers to a molecule having a molecular weight of more than 10,000 Daltons.

The term “polymerization” refers to the process of forming a polymer from monomers. The monomers may be polypeptides, lipids, or amphiphilic molecules that can self-assemble with or without the presence of a template molecule. In this particular case, “polymerization” essentially refers to the self-assembly process. Alternatively, the monomers may be unsaturated molecules that can undergo chain polymerization or copolymerization, or molecules with at least two reactive functional groups that can undergo step polymerization or copolymerization. The unsaturated molecules are exemplified as molecules with vinyl groups, molecules with methacrylate or acrylate groups, molecules with maleic moieties, and other similar unsaturated molecules. In this particular case, “polymerization” refers to the process of chain polymerization or copolymerization. The molecules with at least two reactive functional groups are exemplified as diacids, diamines, diols, dimercaptans, amino acids, monomeric nucleic acids, saccharides, and derivatives thereof.

The term “drug” or “drug molecule” refers to a therapeutic agent including a substance having a beneficial effect on a human or animal body when it is administered to the human or animal body. Preferably, the therapeutic agent includes a substance that can treat, cure or relieve one or more symptoms, illnesses, or abnormal conditions in a human or animal body or enhance the wellness of a human or animal body.

The term “deliver a drug to a particular location in a human or animal body” refers to the process that the drug, which may be encapsulated in a nanoscale delivery vehicle, travels through the organs, fluids or organ components of the human or animal body via the internal digestive system, blood circulation system, fluid circulation system, or external transfer means such as injection, transfusion. The drug reaches the particular location in the body based on a targeting means such as the affinity of the drug to the particular location, the affinity of the delivery vehicle to the particular location, the release tendency of the delivery vehicle at the particular location, controlled release of the drug by the delivery vehicle at the particular location by applying an external stimulus, combinations thereof, and equivalents thereof. The external stimulus may be radiation, chemical stimulation, thermal stimulation, or physical stimulation. Preferably, the external stimulus is targeted to a particular location in the body for maximum effect.

In one embodiment, the polymer of the invention (comprising chimeric polypeptides) or polymer used in the compositions of the invention (drug delivery systems) is a hollow tube having approximately a 25 nm outer diameter and a 20 nm inner diameter. The polymer of the present invention preferably has a bending modulus of 5±2 Gpa. At suitable conditions, the polymer of the invention (comprising chimeric polypeptides) or polymer used in the compositions of the invention (drug delivery systems) may interact with each other by pairing, bundling, entangling (excluded volume interaction) and electrostatic cross-linking (bridging by divalent cations) to form structures varying from a pair of rods to an interconnected network. A transmission electron micrograph of one embodiment of the polymer of the present invention is illustrated in FIG. 1.

In a further aspect, the present invention relates to a method of delivering a drug molecule to a particular location of a human or animal body. According to the present invention, the method of delivering a drug to a particular location of a human or animal body involves: encapsulating the drug molecule with a polymer of the present invention and administering the encapsulated drug molecule to the human or animal body.

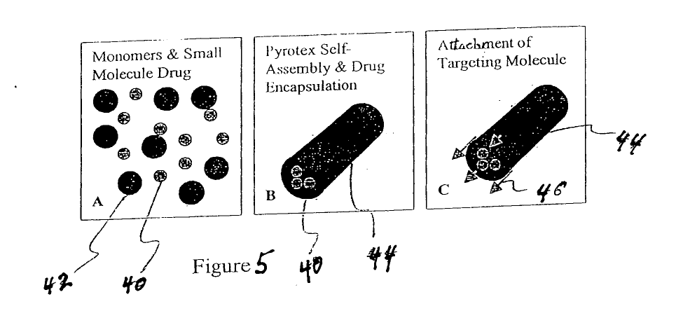

In this method, the encapsulating step may be implemented by forming the polymer in the presence of the drug molecule. Alternatively, the encapsulating step may be implemented by adding the drug molecule to a partially formed polymer and then capping the partially formed polymer using a suitable capping unit such as another monomeric polypeptide unit of the present invention. In another embodiment, the encapsulating step may be carried out by mixing the polymer and the drug molecule together in a solution so that the drug molecule may permeate inside the polymer. In addition, a targeting molecule or vector may be attached to the drug loaded polymer or nanotube during the encapsulation process or after the completion of the encapsulation process. FIG. 2 shows an illustrative diagram of this process. In FIG. 5(A), drug molecules 40 and monomeric polypeptides 42 are dissolved in a solution. In FIG. 5(B), the monomeric polypeptides 40 self-assemble to form a nanoscale polymer 44 encapsulating the drug molecules 40 therein. In FIG. 5(C), targeting vectors 46 are attached to the nanoscale polymer 44.

In another embodiment of encapsulating one or more drugs, in addition to the monomeric polypeptide units, lipids or lipid molecules are used to encapsulate a drug molecule. In this embodiment, liposomes are induced to form from lipids in the presence of both the drug molecules and the monomeric polypeptide units, preferably in a solution, in the presence of a divalent cation such as millimolar calcium and magnesium as described in Akasji et al, Formation of giant liposomes promoted by divalent cations: critical role of electrostatic repulsion, Biophys. J. v. 74, pp. 2973-2982. The formed liposomes encapsulate one or more drug molecules and monomeric polypeptide units therein. After the formation of the liposomes, the condition of the mixture or solution containing the liposomes is changed to, for example, a higher temperature to induce the assembly of the monomeric polypeptide units into polymers or nanotubes to produce a complex wherein the one or more drug molecules are encapsulated in the polymer or nanotube with a lipid coating.

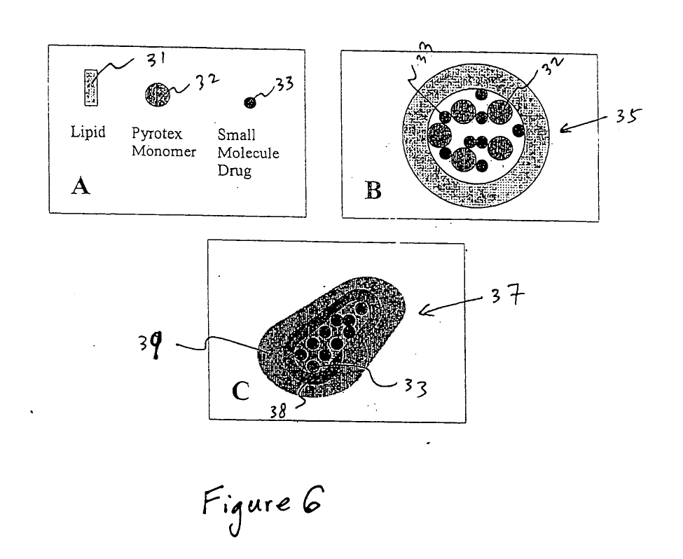

FIGS. 6A, 6B and 6C further illustrate this process. FIG. 6A illustrates a mixture which may contain a plurality of lipids 31, monomeric polypeptide units 32 and drug molecules 33 (only one lipid, monomeric polypeptide unit and drug molecule is actually shown). The mixture forms a complex 35 as shown in FIG. 6B after a suitable period. Complex 35 contains monomeric polypeptide units 32 and drug molecules 33. The complex 35 in FIG. 6B is further converted to an encapsulated drug composition 37 as shown in FIG. 6C after being incubated for a suitable period of time. Encapsulated drug composition 37 contains drug molecules 33, a polymer 38 made from monomeric polypeptide units 32 and a lipid coating 39.

The encapsulated drug molecule may be administered to a human or animal orally, parenterally, by inhalation or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intraveneously.

The drug molecule may be selected from the currently existing drugs and potential future drugs. Preferably, the drug molecule may be selected from those that are harmful to some organs of the body and, therefore, would preferably be delivered only a particular location in the body. The particular location may be a location where an illness is rooted, an infected location, a tumor location, a damaged location, combinations thereof or equivalents thereof.

After the encapsulated drug molecule has been administered, the encapsulated drug molecule within the polymer may travel to the particular location inside the body because of the body fluid circulation, digestion and similar physiological actions. The movement of the encapsulated drug molecule may be further controlled or targeted by one or more targeting vector existed on the surface of the nanoscale polymer or the polymer of the present invention. The movement may be further regulated by one or more external means such as by irradiating the location, or by planting or injecting a receptor. After reaching the desired location, the drug molecule may be released from the polymer based on a condition of the particular location or on an interaction between the polymer and an element of the particular location. The drug release from the polymer may be controlled by a controlling vector on the polymer responsive to an element of the particular location or an external stimulation such as radiation.

According to the present invention, there may be a multitude of applications for the polymer that combines the possibilities of a nanotube with the physical and chemical manipulability of a simple protein structure. The modulus, length, branching, core diameter, core volume, core and surface polarity, thermo- and solvent stability of the polymer may all be varied by means of mutagenesis and directed protein evolution. Furthermore, the amino acid sidechains facing the core and the external solvent may be utilized as reactive groups for controlled addition of chemical substituents. In addition, arrays of photo- or redox-active groups adopting the underlying spiral symmetry provided by the polymer may be light and electron conductive.

Modifications to Improve Protease Resistance of the Monomeric Polypeptide

In one aspect, compositions of the invention (e.g., drug delivery vehicles, fibers) and chimeric polypeptides of the invention use protease resistant cannulae proteins. One of the objectives of improving the protease resistance of the monomeric polypeptide is to increase the time available for drug targeting and drug release at the target site when the polymer containing the monomeric polypeptide is used in a nanoscale drug delivery vehicle or a drug capsule. Improvements in protease resistance may be achieved by several methods. These methods include conventional mutagenesis to remove susceptible cleavage sites, the modification by glycosylation to protect the amino acid backbone of the monomeric polypeptide, and the introduction of poly(ethylene glycol), PEG, to produce a PEGylated monomeric polypeptide that is shielded from proteolysis. The attachment of PEG to the monomeric polypeptide may be achieved through the introduction of surface exposed cysteines that may be used for specific PEG coupling. The modification of the glycosylation pattern and the degree of PEGylation may also depend on other considerations because both modifications have additional benefits as discussed below.

Modifications to Reduce the Immunogenicity of the Monomeric Polypeptide

In one aspect, compositions of the invention (e.g., drug delivery vehicles, fibers) and chimeric polypeptides of the invention use cannulae proteins with reduced immunogenicity. One goal of these modifications is to reduce or mask antigenic determinants on the monomeric polypeptide to minimize potential allergic responses. The method of modifying the monomeric polypeptide involves: analyzing potential antigenic domains, and identifying cysteine insertion sites for possible use in PEGylation masking strategies (see Kozlowski, Harris, Improvements in protein PEGylation: PEGylated interferons for treatment of hepatitis C J. Controlled Release: v. 72, pp. 217-224 (2001)). The method may also involves: computer modeling to identify potential amino acid domains on the monomeric polypeptide surface that are likely to be antigenic followed by modifying these sites through the mutagenesis method described in the present invention. In addition, glycosylation patterns of the monomeric polypeptide may be modified to produce a molecule that is less likely to be recognized as foreign.

Modifications to Attach Targeting Vectors or Molecules on the Monomeric Polypeptide

In one aspect, compositions of the invention (e.g., drug delivery vehicles, fibers) and chimeric polypeptides of the invention use cannulae proteins with modifications to attached to targeting vectors or other targeting molecules. In order to better direct the nanoscale drug delivery vehicle or polymer of the present invention to a particular desired location in an animal body, a targeting vector may be attached to the polymer or the monomeric polypeptide of the present invention. The targeting vector useful in the present invention includes antibodies, oligosaccharides, and Morphatides™. All of these targeting vectors may be readily attached to the monomeric polypeptide surface using conventional chemistries. Antibodies are the most common targeting vectors but oligosaccharides have also been shown to function as effective targeting moieties (see Wu, Evidence for targeted gene delivery to HepG2 hepatoma cells in vitro, V: 27, no. 3, pp. 887-892 (1988); Hashida, Akamatsu, Nishikawa, Fumiyoshi, Takakura, Design of polymeric prodrugs of prostaglandin E₁ having galactose residue for hepatocyte targeting, J. Controlled Release: v. 62, pp. 253-262 (1999)). The presence of a plurality of potential N-linked glycosylation sites in the monomeric polypeptide makes glycosylation-based targeting an attractive approach. In addition, Morphatides™ may be attached to the monomeric polypeptide using common synthetic methods. Morphatides™ is a derivatized nucleotide complex that may be optimized through iterative in vitro evolution to bind specific antigens.

Morphatides™ are evolvable, synthetic molecules that consist of a polynucleotide scaffold in association with reversible modifiers that contribute to molecular selectivity and binding. Morphatides™ possesses both the selective evolvability of aptamers (see Osborne, Ellington, Nucleic Acid Selection and the Challenge of Combinatorial Chemistry. Chemical reviews, v. 97, pp. 349-370 (1997)) and the considerable binding properties of proteins such as demonstrated by antibodies. Morphatides™ are evolvable by repeated cycles of selection against a target molecule. The evolvability of Morphatides™ is made possible in part because the molecular modifications of the polynucleotide scaffold are reversible. This reversibility is an element of their design, because between rounds of affinity selection against a chosen target, the polynucleotide scaffold is subjected to amplification by PCR. An additional feature of the amplified scaffolds in Morphatides™ is their “memory” of which sites were modified so that they may be re-modified for the next round of selection/maturation. Repeated cycles of modification, selection against a chosen target, de-modification and PCR amplification of the selected molecules can thus lead to the enrichment of molecules effectively bred to tightly bind selected targets. Once a Morphatide™ has been successfully evolved against a chosen target, a final Morphatide™ with the desired properties may be produced without the need for reversible chemistry. The final Morphatide™ product is a stable, synthetic, cost-effective molecule with the properties of a synthetic antibody.

Modification to Increase Hydrophobicity of the Interior-Facing Amino Acid Side Chains of the Monomeric Polypeptide

In one aspect, compositions of the invention (e.g., drug delivery vehicles, fibers) and chimeric polypeptides of the invention use cannulae proteins with modifications to increase hydrophobicity, e.g., of the interior facing amino acid side chains of monomeric cannulae proteins. One objective of this modification is to enhance the solubility of encapsulated small molecule drugs that are poorly water-soluble when the monomeric polypeptide polymerizes to form a nanoscale drug capsule or delivery vehicle. Poor water solubility is a frequent drawback for many small molecule drugs (see Muller, Jacobs, Kayser, Nanosuspensions as particulate drug formulations in therapy: Rationale for development and what we can expect for the future, Adv. Drug Delivery Reviews: v. 47, pp. 3-19 (2001)). The monomeric polypeptide may be modified to produce a nanoscale drug encapsulation device that easily transits in an aqueous environment due to its hydrophilic outer surface while maintaining a favorable environment for hydrophobic small drug molecules on its inner surface.

Modification to Vary Drug-Binding Affinity

In one aspect, compositions of the invention (e.g., drug delivery vehicles, fibers) and chimeric polypeptides of the invention use cannulae proteins with modifications for varying drug-binding affinity. A charge environment of a nanoscale drug capsule containing a plurality of the monomeric polypeptide units may affect the rate of drug release. The charge environment may be modified to manipulate the affinity of interactions between the nanoscale drug capsule interior and the encapsulated drug. Changes to the interior that increase the drug affinity of the monomeric polypeptide may lead to slower rates of diffusion and consequently slower rates of drug release. Conversely, changes to the interior that decrease the drug affinity of the monomeric polypeptide may lead to increased rates of drug release.

Modification to Antigenic Domains

In one aspect, compositions of the invention (e.g., drug delivery vehicles, fibers) and chimeric polypeptides of the invention use cannulae proteins with modifications to antigenic domains. For example, a polynucleotide sequence selected from SEQ ID NOS. 1, 3, 5, 7, and 9 and sequences substantially identical or complementary thereto, and fragments thereof may be further modified by incorporating one or more sequences encoding one or more antigens therein using a suitable gene modification method such as recombinant DNA or a method described above. In this method, the one or more sequences encoding one or more antigens are inserted into the polynucleotide sequence so that when the polynucleotide sequence is expressed to produce a polypeptide, the antigen or antigenic domain is exposed on the surface of the expressed polypeptide. In a more preferred embodiment, when expressed polypeptide is assembled or self-assembled into a polymer of the present invention, the antigen or antigenic domain is exposed on the surface of the polymer.

These modifications to the monomeric polypeptide may provide an improved drug delivery vehicle with a prolonged circulation lifespan, capable of controlled release of its contents at specific target sites.

Generating and Manipulating Nucleic Acids

The invention provides nucleic acids, including expression cassettes such as expression vectors, encoding cannulae proteins, such as CanA, CanB, CanC, CanD or CanE including, e.g., the exemplary polypeptides SEQ ID NO:2, SEQ ID NO:4, SEQ ID NO:6, SEQ ID NO:8 or SEQ ID NO:10, and sequence substantially identical thereto, for using in the chimeric polypeptides of the invention and drug delivery systems of the invention. The invention also includes methods for modifying nucleic acids encoding the cannulae proteins used in the compositions of the invention, e.g., drug delivery systems of the invention, or the chimeric polypeptides of the invention by, e.g., synthetic ligation reassembly, optimized directed evolution system and/or saturation mutagenesis, such as Gene Site Saturation Mutagenesis™ (GSSM™) and GeneReassembly™.

The nucleic acids of the invention can be made, isolated and/or manipulated by, e.g., cloning and expression of cDNA libraries, amplification of message or genomic DNA by PCR, and the like. The invention can be practiced in conjunction with any method or protocol or device known in the art, which are well described in the scientific and patent literature.

General Techniques

The nucleic acids used to practice this invention, whether RNA, iRNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice the invention is to clone from genomic samples, and, if desired, screen and re-clone inserts isolated or amplified from, e.g., genomic clones or cDNA clones. Sources of nucleic acid used in practicing the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In one aspect, a nucleic acid encoding a polypeptide of the invention is assembled in appropriate phase with a leader sequence capable of directing secretion of the translated polypeptide or fragment thereof.

The invention provides fusion proteins and nucleic acids encoding them. In addition to chiral selection motifs, enzymes, receptors, ligands, antibodies, antigens, epitopes and the like, polypeptide of the invention can be fused to a heterologous peptide or polypeptide such as N-terminal identification peptide, which imparts desired characteristics such as increased stability or simplified purification. Peptides and polypeptides of the invention also can be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAG™ (a FLAG sequence of amino acids) extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein.

Transcriptional and Translational Control Sequences

The invention provides nucleic acid (e.g., DNA) sequences of the invention operatively linked to expression (e.g., transcriptional or translational) control sequence(s), e.g., promoters or enhancers, to direct or modulate RNA synthesis/expression. The expression control sequence can be in an expression vector. Exemplary bacterial promoters include lacI, lacZ, T3, T7, gpt, lambda PR, PL and trp. Exemplary eukaryotic promoters include CMV immediate early, HSV thymidine kinase, early and late SV40, LTRs from retrovirus, and mouse metallothionein I.

Promoters suitable for expressing a polypeptide in bacteria include the E. coli lac or trp promoters, the lacI promoter, the lacZ promoter, the T3 promoter, the T7 promoter, the gpt promoter, the lambda PR promoter, the lambda PL promoter, promoters from operons encoding glycolytic enzymes such as 3-phosphoglycerate kinase (PGK), and the acid phosphatase promoter. Eukaryotic promoters include the CMV immediate early promoter, the HSV thymidine kinase promoter, heat shock promoters, the early and late SV40 promoter, LTRs from retroviruses, and the mouse metallothionein-I promoter. Other promoters known to control expression of genes in prokaryotic or eukaryotic cells or their viruses may also be used.

The invention provides expression cassettes that can be expressed in a tissue-specific manner, e.g., that can express a chimeric polypeptide of the invention in a tissue-specific manner. The invention provides plants or seeds that express a chimeric polypeptide of the invention in a tissue-specific manner. The tissue-specificity can be seed specific, stem specific, leaf specific, root specific, fruit specific and the like. The nucleic acids of the invention can also be operably linked to plant promoters which are inducible upon exposure to chemicals reagents.

Expression Vectors and Cloning Vehicles

The invention provides expression vectors and cloning vehicles comprising nucleic acids of the invention, e.g., sequences encoding the chimeric polypeptides of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available. Exemplary vectors are include: bacterial: pQE vectors (Qiagen), pBluescript plasmids, pNH vectors, (lambda-ZAP vectors (Stratagene); ptrc99a, pKK223-3, pDR540, pRIT2T (Pharmacia); Eukaryotic: pXT1, pSG5 (Stratagene), pSVK3, pBPV, pMSG, pSVLSV40 (Pharmacia). However, any other plasmid or other vector may be used so long as they are replicable and viable in the host. Low copy number or high copy number vectors may be employed with the present invention.

The expression vector can comprise a promoter, a ribosome binding site for translation initiation and a transcription terminator. The vector may also include appropriate sequences for amplifying expression. Mammalian expression vectors can comprise an origin of replication, any necessary ribosome binding sites, a polyadenylation site, splice donor and acceptor sites, transcriptional termination sequences, and 5′ flanking non-transcribed sequences. In some aspects, DNA sequences derived from the SV40 splice and polyadenylation sites may be used to provide the required non-transcribed genetic elements.

In one aspect, the expression vectors contain one or more selectable marker genes to permit selection of host cells containing the vector. Such selectable markers include genes encoding dihydrofolate reductase or genes conferring neomycin resistance for eukaryotic cell culture, genes conferring tetracycline or ampicillin resistance in E. coli, and the S. cerevisiae TRP1 gene. Promoter regions can be selected from any desired gene using chloramphenicol transferase (CAT) vectors or other vectors with selectable markers.

Vectors for expressing the polypeptide or fragment thereof in eukaryotic cells can also contain enhancers to increase expression levels. Examples include the SV40 enhancer on the late side of the replication origin bp 100 to 270, the cytomegalovirus early promoter enhancer, the polyoma enhancer on the late side of the replication origin, and the adenovirus enhancers.

A nucleic acid sequence can be inserted into a vector by a variety of procedures. In general, the sequence is ligated to the desired position in the vector following digestion of the insert and the vector with appropriate restriction endonucleases. Alternatively, blunt ends in both the insert and the vector may be ligated. A variety of cloning techniques are known in the art, e.g., as described in Ausubel and Sambrook. Such procedures and others are deemed to be within the scope of those skilled in the art.

The vector can be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook. Any vector may be used as long as it is replicable and viable in the host cell.

The nucleic acids of the invention can be expressed in expression cassettes, vectors or viruses and transiently or stably expressed in plant cells and seeds. One exemplary transient expression system uses episomal expression systems, e.g., cauliflower mosaic virus (CaMV) viral RNA generated in the nucleus by transcription of an episomal mini-chromosome containing supercoiled DNA, see, e.g., Covey (1990) Proc. Natl. Acad. Sci. USA 87:1633-1637. Alternatively, coding sequences, i.e., all or sub-fragments of sequences of the invention can be inserted into a plant host cell genome becoming an integral part of the host chromosomal DNA. Sense or antisense transcripts can be expressed in this manner. A vector comprising the sequences (e.g., promoters or coding regions) from nucleic acids of the invention can comprise a marker gene that confers a selectable phenotype on a plant cell or a seed. For example, the marker may encode biocide resistance, particularly antibiotic resistance, such as resistance to kanamycin, G418, bleomycin, hygromycin, or herbicide resistance, such as resistance to chlorosulfuron or Basta.

Expression vectors capable of expressing nucleic acids and proteins in plants are well known in the art, and can include, e.g., vectors from Agrobacterium spp., potato virus X (see, e.g., Angell (1997) EMBO J. 16:3675-3684), tobacco mosaic virus (see, e.g., Casper (1996) Gene 173:69-73), tomato bushy stunt virus (see, e.g., Hillman (1989) Virology 169:42-50), tobacco etch virus (see, e.g., Dolja (1997) Virology 234:243-252), bean golden mosaic virus (see, e.g., Morinaga (1993) Microbiol Immunol. 37:471-476), cauliflower mosaic virus (see, e.g., Cecchini (1997) Mol. Plant Microbe Interact. 10:1094-1101), maize Ac/Ds transposable element (see, e.g., Rubin (1997) Mol. Cell. Biol. 17:6294-6302; Kunze (1996) Curr. Top. Microbiol. Immunol. 204:161-194), and the maize suppressor-mutator (Spm) transposable element (see, e.g., Schlappi (1996) Plant Mol. Biol. 32:717-725); and derivatives thereof.

In one aspect, the expression vector can have two replication systems to allow it to be maintained in two organisms, for example in mammalian or insect cells for expression and in a prokaryotic host for cloning and amplification. Furthermore, for integrating expression vectors, the expression vector can contain at least one sequence homologous to the host cell genome. It can contain two homologous sequences which flank the expression construct. The integrating vector can be directed to a specific locus in the host cell by selecting the appropriate homologous sequence for inclusion in the vector. Constructs for integrating vectors are well known in the art.

Expression vectors of the invention may also include a selectable marker gene to allow for the selection of bacterial strains that have been transformed, e.g., genes which render the bacteria resistant to drugs such as ampicillin, chloramphenicol, erythromycin, kanamycin, neomycin and tetracycline. Selectable markers can also include biosynthetic genes, such as those in the histidine, tryptophan and leucine biosynthetic pathways.

The terms “vector” and “expression cassette” as used herein can be used interchangeably and refer to a nucleotide sequence which is capable of affecting expression of a nucleic acid, e.g., a mutated nucleic acid of the invention. Expression cassettes can include at least a promoter operably linked with the polypeptide coding sequence; and, optionally, with other sequences, e.g., transcription termination signals. Additional factors necessary or helpful in effecting expression may also be used, e.g., enhancers. “Operably linked” as used herein refers to linkage of a promoter upstream from a DNA sequence such that the promoter mediates transcription of the DNA sequence. Thus, expression cassettes also include plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and includes both the expression and non-expression plasmids.

Vectors for expressing cannulae proteins used in the drug delivery systems of the invention, or the chimeric polypeptides of the invention, can be selected from many known vectors such as the one contained in plasmid pEX-CAN-A, which is described in detail by B, Mai et al in Mai, Frey, Swanson, Mathur, Stetter, Molecular Cloning and Functional Expression of a Protein-Serine/Threonine Phosphatase from the hyperthermophilic Archaeon Archaeon abyssi TAG11. J. Bacterial. (1998) 180:4030-4035, pBluescript II phagemid KS(−), pET17b and a suitable virus. The vector that can be used in the present invention can be selected from a vector listed:

| PLASMID | SIZE | PROPERTY |
|---|---|---|
| pBluescript ® II | 2.96 kb | AmpR; MCS flanked by T3 and |
| phagemid KS(-) | | T7 promoter; replication vector |
| pET17b | 3.31 kb | AmpR; MCS flanked by T7 promoter |
| | | and T7 terminator; expression vector |

Host Cells and Transformed Cells

The invention also provides a transformed cell comprising a nucleic acid sequence of the invention, e.g., a sequence encoding chimeric polypeptides of the invention, or an expression cassette, e.g., a vector, of the invention. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art. Techniques for transforming a wide variety of higher plant species are well known and described in the technical and scientific literature. See, e.g., Weising (1988) Ann. Rev. Genet. 22:421-477, U.S. Pat. No. 5,750,870.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)).

In one aspect, the nucleic acids or vectors of the invention are introduced into the cells for screening, thus, the nucleic acids enter the cells in a manner suitable for subsequent expression of the nucleic acid. The method of introduction is largely dictated by the targeted cell type. Exemplary methods include CaPO₄ precipitation, liposome fusion, lipofection (e.g., LIPOFECTIN™), electroporation, viral infection, etc. The candidate nucleic acids may stably integrate into the genome of the host cell (for example, with retroviral introduction) or may exist either transiently or stably in the cytoplasm (i.e. through the use of traditional plasmids, utilizing standard regulatory sequences, selection markers, etc.). As many pharmaceutically important screens require human or model mammalian cell targets, retroviral vectors capable of transfecting such targets are preferred.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

In one aspect, in a second step of the process, a vector with the predetermined nucleic acid attached is inserted or implanted into a host cell using any method known to a skilled person in the art. The host cell may be an E. coli cell, a fungus cell, a cancer cell, a Pyrodictium abyssi cell, a hyperthermus butylicus cell, Pseudomonas or any other suitable prokaryotic or eukaryotic cells. In one aspect, the host cell used in the invention is selected from an organism listed below, e.g., the host cell is E. coli BL21 (DE3).

| Organism | Reference |
|---|---|
| Pyrodictium abyssi | Deininger W., 1994 |
| isolate TAG11 | |
| Hyperthermus butylicus | Zillig et al., 1990; DSMZ 5456 |
| E. coli DH5α | Woodcock et al., 1989; |
| | [Stratagene, Heidelberg] |
| E. coli Y1090 | Young and Davis, 1983; |
| | [Stratagene, Heidelberg] |
| E. coli BL 21 (DE3) | Phillips et al., 1984; |
| | [Stratagene, Heidelberg] |

Alternatively, the host cell used in the present invention may be a plant cell so that the plant may be able to over express the nucleic acid to produce the monomeric polypeptide of the present invention.

In one aspect, in a third step of the process, the gene represented by the predetermined nucleic acid is expressed in the host cell under suitable conditions such as by employing a suitable culture or medium. During this third step of the process, the host cell may replicate itself to produce additional host cells containing the same vectors therein. A suitable culture media and suitable conditions for expression of Pyrodictium abyssi are described below.

Medium for Pyrodictium abyssi (pH 5.5-6.0)

| | | |
|---|---|---|
| SME | 500.00 | ml |
| KH₂PO₄ | 0.50 | g |
| Yeast extract | 0.50 | g |
| Na₂S₂O₃ | 1.00 | g |
| Resazurin (1%) | 0.30 | ml |

| | |
|---|---|
| H₂O_{bidist} | up to 1,000.00 ml |

The medium was autoclaved. The cultivation temperature was 102° C. The host cell was incubated while standing. “SME” stands for Synthetic Sea Water, which is typically prepared using the procedure described in Example 1. A suitable media and suitable conditions for expression of Hyperthermus butylicus are described below.

Medium for Hyperthermus butylicus (pH 7.0)

| | | |
|---|---|---|
| SME | 500.00 | ml |
| KH₂PO₄ | 0.50 | g |
| NH₄Cl | 0.50 | g |
| Sulfur | 5.00 | g |
| KJ | 2.50 | mg |
| NiSO₄ × 6 H₂O | 2.00 | mg |
| Resazurin (1%) | 0.30 | ml |

| | |
|---|---|
| H₂O_{bidist} | up to 1,000.00 ml |

The medium was vaporized. Prior to inoculation, 6 g tryptone per liter were added in the form of an autoclaved stock solution (10%, w/v). The cultivation temperature was 100° C. The host cell was incubated while standing.

Exemplary media for E. coli are described as follows. E. coli strains were routinely cultivated aerobically on LB₀ medium (see below) at 37° C. with intensive shaking (250 rpm). Plasmid-carrying or vector-carrying strains with resistance to antibiotics were cultivated in the presence of the corresponding antibiotic (100 μg/ml) ampicillin, 34 μg/ml chloramphenicol).

LB₀ Medium for E. coli DH5α and BL 21 (DE3), (pH 7.0)

| | | |
|---|---|---|
| Tryptone | 10.00 | g |
| Yeast extract | 5.00 | g |
| NaCl | 10.00 | g |

| | |
|---|---|
| H₂O_{bidist} | up to 1,000 ml |

LB₀ Medium for E. coli Y1090 (pH 7.0)

| | | |
|---|---|---|
| Tryptone | 10.00 | g |
| Yeast extract | 10.00 | g |
| NaCl | 5.00 | g |

| | |
|---|---|
| H₂O_{bidist} | up to 1,000 ml |

NZYM Medium for E. coli Y1090 (pH 7.0)

| | | |
|---|---|---|
| NZ amines | 10.00 | g |
| NaCl | 5.00 | g |
| Yeast extract | 5.00 | g |
| MgSO₄ × 7 H₂O | 2.00 | g |

| | |
|---|---|
| H₂O_{bidist} | up to 1,000 ml |

For the preparation of plates, 15 g agar per liter of medium was used. Added to the Top Agar were 7.5 g agarose per liter medium. Exemplary conditions for expressing the gene encoded by the nucleic acid used in the present invention involve: keeping the medium at 37° C. under aeration in a fermentor, stirring the medium containing the E. coli cells, and inducing the gene overexpression by adding IPTG.

In one embodiment, the process of preparing monomeric polypeptides or polypeptide units of the present invention further includes a fourth step of isolating the produced polypeptide from the culture or medium. The step of isolating the monomeric polypeptide can be carried out by French pressing the E. coli cell mass from a solution, removing particles from the solution by centrifugation, heat-treating the solution to precipitate the unwanted heat-sensitive proteins, centrifugating the heat-treated solution to obtain a clear solution, precipitating the monomeric polypeptides from the clear solution using ammonium sulfate and dialyzing the monomeric polypeptides to reduce the ionic strength of the solution.

Amplification of Nucleic Acids

In practicing the invention, nucleic acids of the invention and nucleic acids encoding the chimeric polypeptides of the invention, or modified nucleic acids of the invention, can be reproduced by amplification. Amplification can also be used to clone or modify the nucleic acids of the invention. Thus, the invention provides amplification primer sequence pairs for amplifying nucleic acids of the invention.

Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified.

The skilled artisan can select and design suitable oligonucleotide amplification primers. Amplification methods are also well known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

Hybridization of Nucleic Acids

Cannulae polypeptides used in the drug delivery systems and chimeric polypeptides of the invention also include “substantially identical” polypeptides encoded by nucleic acids that hybridize under defined conditions, defined below (e.g., under various stringency—high, medium, (reduced) low—conditions), to the exemplary SEQ ID NO:1, SEQ ID NO:3, SEQ ID NO:5, SEQ ID NO:7. “Hybridization” refers to the process by which a nucleic acid strand joins with a complementary strand through base pairing. Hybridization reactions can be sensitive and selective so that a particular sequence of interest can be identified even in samples in which it is present at low concentrations. Suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature.

For example, hybridization under high stringency conditions could occur in about 50% formamide at about 37° C. to 42° C. Hybridization could occur under reduced stringency conditions in about 35% to 25% formamide at about 30° C. to 35° C. In particular, hybridization could occur under high stringency conditions at 42° C. in 50% formamide, 5×SSPE, 0.3% SDS, and 200 ng/ml sheared and denatured salmon sperm DNA. Hybridization could occur under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

Hybridization may be carried out under conditions of low stringency, moderate stringency or high stringency. As an example of nucleic acid hybridization, a polymer membrane containing immobilized denatured nucleic acids is first prehybridized for 30 minutes at 45° C. in a solution consisting of 0.9 M NaCl, 50 mM NaH₂PO₄, pH 7.0, 5.0 mM Na₂EDTA, 0.5% SDS, 10× Denhardt's, and 0.5 mg/ml polyriboadenylic acid. Approximately 2×10⁷ cpm (specific activity 4-9×10⁸ cpm/1 g) of ³²P end-labeled oligonucleotide probe are then added to the solution. After 12-16 hours of incubation, the membrane is washed for 30 minutes at room temperature in 1×SET (150 mM NaCl, 20 mM TRIS® (hydroxymethyl amino methane hydrochloride), pH 7.8, 1 mM Na₂EDTA) containing 0.5% SDS, followed by a 30 minute wash in fresh 1×SET at Tm-10° C. for the oligonucleotide probe. The membrane is then exposed to auto-radiographic film for detection of hybridization signals.

By varying the stringency of the hybridization conditions used to identify nucleic acids, such as cDNAs or genomic DNAs, which hybridize to the detectable probe, nucleic acids having different levels of homology to the probe can be identified and isolated. Stringency may be varied by conducting the hybridization at varying temperatures below the melting temperatures of the probes. The melting temperature, Tm, is the temperature (under defined ionic strength and pH) at which 50% of the target sequence hybridizes to a perfectly complementary probe. Very stringent conditions are selected to be equal to or about 5° C. lower than the Tm for a particular probe. The melting temperature of the probe may be calculated using the following formulas:

For probes between 14 and 70 nucleotides in length the melting temperature (Tm) is calculated using the formula: Tₘ=81.5+16.6(log [Na⁺])+0.41 (fraction G+C)−(600/N) where N is the length of the probe.

If the hybridization is carried out in a solution containing formamide, the melting temperature may be calculated using the equation: Tₘ=81.5+16.6(log [Na⁺])+0.41 (fraction G+C)−(0.63% formamide)−(600/N) where N is the length of the probe.

Prehybridization may be carried out in 6×SSC, 5× Denhardt's reagent, 0.5% SDS, 100 μg denatured fragmented salmon sperm DNA or 6×SSC, 5× Denhardt's reagent, 0.5% SDS, 100 μg denatured fragmented salmon sperm DNA, 50% formamide. The formulas for SSC and Denhardt's solutions are listed in Sambrook et al., supra.

Hybridization is conducted by adding the detectable probe to the prehybridization solutions listed above. Where the probe comprises double stranded DNA, it is denatured before addition to the hybridization solution. The filter is contacted with the hybridization solution for a sufficient period of time to allow the probe to hybridize to cDNAs or genomic DNAs containing sequences complementary thereto or homologous thereto. For probes over 200 nucleotides in length, the hybridization may be carried out at 15-25° C. below the Tm. For shorter probes, such as oligonucleotide probes, the hybridization may be conducted at 5-10° C. below the Tm. Typically, for hybridizations in 6×SSC, the hybridization is conducted at approximately 68° C. Usually, for hybridizations in 50% formamide containing solutions, the hybridization is conducted at approximately 42° C.

All of the foregoing hybridizations would be considered to be under conditions of high stringency.

Following hybridization, the filter is washed to remove any non-specifically bound detectable probe. The stringency used to wash the filters can also be varied depending on the nature of the nucleic acids being hybridized, the length of the nucleic acids being hybridized, the degree of complementarity, the nucleotide sequence composition (e.g., GC v. AT content), and the nucleic acid type (e.g., RNA v. DNA). Examples of progressively higher stringency condition washes are as follows: 2×SSC, 0.1% SDS at room temperature for 15 minutes (low stringency); 0.1×SSC, 0.5% SDS at room temperature for 30 minutes to 1 hour (moderate stringency); 0.1×SSC, 0.5% SDS for 15 to 30 minutes at between the hybridization temperature and 68° C. (high stringency); and 0.15M NaCl for 15 minutes at 72° C. (very high stringency). A final low stringency wash can be conducted in 0.1×SSC at room temperature. The examples above are merely illustrative of one set of conditions that can be used to wash filters. One of skill in the art would know that there are numerous recipes for different stringency washes. Some other examples are given below.

Nucleic acids which have hybridized to the probe are identified by autoradiography or other conventional techniques.

The above procedure may be modified to identify nucleic acids having decreasing levels of homology to the probe sequence. For example, to obtain nucleic acids of decreasing homology to the detectable probe, less stringent conditions may be used. For example, the hybridization temperature may be decreased in increments of 5° C. from 68° C. to 42° C. in a hybridization buffer having a Na⁺ concentration of approximately 1M. Following hybridization, the filter may be washed with 2×SSC, 0.5% SDS at the temperature of hybridization. These conditions are considered to be “moderate” conditions above 50° C. and “low” conditions below 50° C. A specific example of “moderate” hybridization conditions is when the above hybridization is conducted at 55° C. A specific example of “low stringency” hybridization conditions is when the above hybridization is conducted at 45° C.

Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

For example, the preceding methods may be used to isolate nucleic acids having a sequence with at least about 97%, at least 95%, at least 90%, at least 85%, at least 80%, at least 75%, at least 70%, at least 65%, at least 60%, at least 55% or at least 50% sequence identity (homology) to a cannulae-encoding nucleic acid sequence (e.g., SEQ ID NO:1, SEQ ID NO:3, SEQ ID NO:5, SEQ ID NO:7), sequences substantially identical thereto, or fragments comprising at least about 10, 15, 20, 25, 30, 35, 40, 50, 75, 100, 150, 200, 300, 400, or 500 consecutive bases thereof, and the sequences complementary to any of the foregoing sequences. Homology (sequence identity) may be measured using an alignment algorithm (see below). For example, the homologous polynucleotides may have a coding sequence which is a naturally occurring allelic variant of one of the coding sequences described herein. Such allelic variants may have a substitution, deletion or addition of one or more nucleotides when compared to a cannulae-encoding nucleic acid sequence, or sequences complementary thereto.

Additionally, the above procedures may be used to isolate nucleic acids which encode polypeptides having at least about 99%, at least 95%, at least 90%, at least 85%, at least 80%, at least 75%, at least 70%, at least 65%, at least 60%, at least 55% or at least 50% homology to an exemplary cannulae sequences (e.g., SEQ ID NO:2, SEQ ID NO:4, SEQ ID NO:6, SEQ ID NO:8), sequences substantially identical thereto, or fragments comprising at least 5, 10, 15, 20, 25, 30, 35, 40, 50, 75, 100, or 150 consecutive amino acids thereof as determined using a sequence alignment algorithm (e.g., such as the FASTA version 3.0t78 algorithm with the default parameters).

Determining the Degree of Sequence Identity

The cannulae polypeptide can comprise a protein having at least about 55%, 60%, 65%, 70%, 75%, 80%, 85%, 90%, 95%, 98%, 99%, or more, sequence identity to SEQ ID NO:2, SEQ ID NO:4, or SEQ ID NO:6, and is capable of assembling into a polymer, e.g., a nanotubule, or, is capable of acting as a chiral selector. The chimeric cannulae proteins can assemble into nanotubular polymers to act as a chiral selectors, biosynthetic pathways, selection scaffoldings and the like. The extent of sequence identity (homology) may be determined using any computer program and associated parameters, including those described herein, such as BLAST 2.2.2. or FASTA version 3.0t78, with the default parameters.

Various sequence comparison programs identified herein are used in this aspect of the invention. Protein and/or nucleic acid sequence identities (homologies) may be evaluated using any of the variety of sequence comparison algorithms and programs known in the art. Such algorithms and programs include, but are not limited to, TBLASTN, BLASTP, FASTA, TFASTA, and CLUSTALW (Pearson and Lipman, Proc. Natl. Acad. Sci. USA 85(8):2444-2448, 1988; Altschul et al., J. Mol. Biol. 215(3):403-410, 1990; Thompson et al., Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993).

Homology or identity can be measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence (a sequence of the invention to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the numbers of contiguous residues. For example, in alternative aspects of the invention, contiguous residues ranging anywhere from 20 to the full length of an exemplary polypeptide or nucleic acid sequence of the invention are compared to a reference sequence of the same number of contiguous positions after the two sequences are optimally aligned. If the reference sequence has the requisite sequence identity to an exemplary polypeptide or nucleic acid sequence of the invention, e.g., 50%, 55%, 60%, 65%, 70%, 75%, 80%, 90% or 95%, 98%, 99% or more sequence identity to a cannulae polypeptide, that sequence may be within the scope of the invention. In alternative embodiments, subsequences ranging from about 20 to 600, about 50 to 200, and about 100 to 150 are compared to a reference sequence of the same number of contiguous positions after the two sequences are optimally aligned. Methods of alignment of sequence for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of person & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. Other algorithms for determining homology or identity include, for example, in addition to a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF. Such alignment programs can also be used to screen genome databases to identify polynucleotide sequences having substantially identical sequences. A number of genome databases are available, for example, a substantial portion of the human genome is available as part of the Human Genome Sequencing Project (Gibbs, 1995). Databases containing genomic information annotated with some functional information are maintained by different organization, and are accessible via the internet.

BLAST, BLAST 2.0 and BLAST 2.2.2 algorithms are also used to practice the invention. They are described, e.g., in Altschul (1977) Nuc. Acids Res. 25:3389-3402; Altschul (1990) J. Mol. Biol. 215:403-410. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990) supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectations (E) of 10, and the BLOSUM62 scoring matrix (see Henikoff & Henikoff (1989) Proc. Natl. Acad. Sci. USA 89:10915) alignments (B) of 50, expectation (E) of 10, M=5, N=−4, and a comparison of both strands. The BLAST algorithm also performs a statistical analysis of the similarity between two sequences (see, e.g., Karlin & Altschul (1993) Proc. Natl. Acad. Sci. USA 90:5873). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001. In one aspect, protein and nucleic acid sequence homologies are evaluated using the Basic Local Alignment Search Tool (“BLAST”). For example, five specific BLAST programs can be used to perform the following task: (1) BLASTP and BLAST3 compare an amino acid query sequence against a protein sequence database; (2) BLASTN compares a nucleotide query sequence against a nucleotide sequence database; (3) BLASTX compares the six-frame conceptual translation products of a query nucleotide sequence (both strands) against a protein sequence database; (4) TBLASTN compares a query protein sequence against a nucleotide sequence database translated in all six reading frames (both strands); and, (5) TBLASTX compares the six-frame translations of a nucleotide query sequence against the six-frame translations of a nucleotide sequence database. The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., Science 256:1443-1445, 1992; Henikoff and Henikoff, Proteins 17:49-61, 1993). Less preferably, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation).

In one aspect of the invention, to determine if a nucleic acid has the requisite sequence identity to be within the scope of the invention, the NCBI BLAST 2.2.2 programs is used, default options to blastp. There are about 38 setting options in the BLAST 2.2.2 program. In this exemplary aspect of the invention, all default values are used except for the default filtering setting (i.e., all parameters set to default except filtering which is set to OFF); in its place a “−F F” setting is used, which disables filtering. Use of default filtering often results in Karlin-Altschul violations due to short length of sequence.

The default values used in this exemplary aspect of the invention include:
“Filter for low complexity: ON
Word Size: 3
Matrix: Blosum62
Gap Costs: Existence: 11
Extension: 1”

Other default settings can be: filter for low complexity OFF, word size of 3 for protein, BLOSUM62 matrix, gap existence penalty of −11 and a gap extension penalty of −1. An exemplary NCBI BLAST 2.2.2 program setting has the “−W” option default to 0. This means that, if not set, the word size defaults to 3 for proteins and 11 for nucleotides.

Modification of Nucleic Acids

The invention provides methods of generating variants of the nucleic acids encoding the chimeric polypeptides of the invention and cannulae proteins used in the drug delivery systems of the invention. These methods can be repeated or used in various combinations to generate chimeric polypeptides having an altered or different activity or an altered or different stability from that of a chimeric polypeptide encoded by the template nucleic acid. These methods also can be repeated or used in various combinations, e.g., to generate variations in gene/message expression, message translation or message stability. In another aspect, the genetic composition of a cell is altered by, e.g., modification of a homologous gene ex vivo, followed by its reinsertion into the cell.

The invention provides methods for evolving enzymes in vitro or in vivo to produce variants with characteristics tailored for specific applications. For example, using the evolution strategies of the invention, enzyme active sites can be modified to produce proteins that retain stereospecific substrate recognition but lack catalytic activity. In one aspect, the chimeric monomers and polymers of the invention are evolved for applications in chiral selection using targeted mutagenesis and in vitro evolution strategies, e.g., as described herein, such as Gene Site Saturation Mutagenesis™ (GSSM™) and GeneReassembly™ (see, e.g., U.S. Pat. Nos. 6,171,820, and 5,965,408 respectively). These technologies are used to create large libraries of mutagenized sequence variants that are screened in a high throughput (HT) assay that selects mutants with a desired phenotype.

With GSSM™, the effects of all 64 codons (even nonsense codons) can be tested at each triplet position along the entire length of the open reading frame of the gene being analyzed. For example, in the case of a 200 amino acid protein, the gene can be simultaneously assembled in 200 different reaction tubes where all 64 codons are present during the synthesis of each amino acid. The result is a library of single point mutants with all possible codons represented at each position of the open reading frame. The library of GSSM™ variants then can be screened using a HT assay to identify variants that have evolved the target phenotype. Individual GSSM™ variants that exhibit the desired property then can be further evolved using GeneReassembly™.

In GeneReassembly™, a new library of mutants can be constructed by recombining DNA fragments taken from the single point mutant sequences identified in the GSSM™ screen. Therefore, the reassembly library can contain open reading frames that contain multiple point mutations that have accumulated as a result of the recombination process. The reassembled variants can be screened to identify mutant combinations with further improvements in the target activity. If necessary, GeneReassembly™ can be repeated until an evolved protein with the desired target properties is identified. These protein evolution strategies do not require prior knowledge of protein structure and therefore produce unbiased pools of protein variants for screening.

In one aspect, the invention provides combinatorial approaches to chiral selector methods. For example, high throughput screening methods of the invention can be used to screen libraries of peptides to identify those sequences with unique enantio-recognition properties; see, e.g., Chankvetadze (2001) supra. Thus, the invention provides chimeric monomers and polymers, including nanotubules, comprising libraries of peptides. In one aspect, these peptide sequences are inserted into the sequence of a chimeric monomer and uniformly displayed on the nanotubule surface.

In one aspect, to apply evolution technologies to the development of chiral selectors, the invention provides a high throughput screen suitable for the identification of protein variants that possess increased enantioselectivity. For example, Henriksson (1996) supra, have reported that the activity of cellobiohydrolase (CBH) from Trichoderma reesei is differentially inhibited by the (R)- and (S)-enantiomers of the beta-blockers propanolol and alprenolol. The T. reesei CBH has been demonstrated to be an effective chiral selector for beta-blockers and the chiral selectivity is consistent with the inhibition data. Based on these results, the methods of the invention evolve the enantioselectivity of CBH using evolution strategies. In one aspect, a high throughput screen is used that measures enantiospecific inhibition of CBH activity.

In practicing the invention, a nucleic acid (e.g., a nucleic acid encoding, e.g., cannulae proteins used in the drug delivery systems of the invention, or, chimeric proteins of the invention) can be altered by any means. For example, random or stochastic methods, or, non-stochastic, or “directed evolution,” methods, see, e.g., U.S. Pat. No. 6,361,974. Methods for random mutation of genes are well known in the art, see, e.g., U.S. Pat. No. 5,830,696. For example, mutagens can be used to randomly mutate a gene. Mutagens include, e.g., ultraviolet light or gamma irradiation, or a chemical mutagen, e.g., mitomycin, nitrous acid, photoactivated psoralens, alone or in combination, to induce DNA breaks amenable to repair by recombination. Other chemical mutagens include, for example, sodium bisulfite, nitrous acid, hydroxylamine, hydrazine or formic acid. Other mutagens are analogues of nucleotide precursors, e.g., nitrosoguanidine, 5-bromouracil, 2-aminopurine, or acridine. These agents can be added to a PCR reaction in place of the nucleotide precursor thereby mutating the sequence. Intercalating agents such as proflavine, acriflavine, quinacrine and the like can also be used.

Any technique in molecular biology can be used, e.g., random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196, to generate variant cannulae proteins used in the drug delivery systems of the invention, or, chimeric proteins of the invention. Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793. In alternative aspects, modifications, additions or deletions are introduced by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, Gene Site Saturated Mutagenesis™ (GSSM™), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods.

The following publications describe a variety of recursive recombination procedures and/or methods which can be used to generate variant polypeptides used to practice the invention (e.g., cannulae proteins used in the drug delivery systems of the invention, or, chimeric proteins of the invention): Stemmer (1999) “Molecular breeding of viruses for targeting and other clinical properties” Tumor Targeting 4:1-4; Ness (1999) Nature Biotechnology 17:893-896; Chang (1999) “Evolution of a cytokine using DNA family shuffling” Nature Biotechnology 17:793-797; Minshull (1999) “Protein evolution by molecular breeding” Current Opinion in Chemical Biology 3:284-290; Christians (1999) “Directed evolution of thymidine kinase for AZT phosphorylation using DNA family shuffling” Nature Biotechnology 17:259-264; Crameri (1998) “DNA shuffling of a family of genes from diverse species accelerates directed evolution” Nature 391:288-291; Crameri (1997) “Molecular evolution of an arsenate detoxification pathway by DNA shuffling,” Nature Biotechnology 15:436-438; Zhang (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp. 447-457; Crameri and Stemmer (1995) “Combinatorial multiple cassette mutagenesis creates all the permutations of mutant and wildtype cassettes” BioTechniques 18:194-195; Stemmer et al. (1995) “Single-step assembly of a gene and entire plasmid form large numbers of oligodeoxyribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating variant sequences in practicing the invention include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using Ml 3-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional protocols for generating variant sequences in practicing the invention include point mismatch repair (Kramer (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundstrom et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

Additional protocols for generating variant sequences in practicing the invention include those discussed in U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Additional protocols for generating variant sequences in practicing the methods of the invention are described in U.S. patent application Ser. No. 09/407,800, “SHUFFLING OF CODON ALTERED GENES” by Patten et al. filed Sep. 28, 1999; “EVOLUTION OF WHOLE CELLS AND ORGANISMS BY RECURSIVE SEQUENCE RECOMBINATION” by del Cardayre et al., U.S. Pat. No. 6,379,964; “OLIGONUCLEOTIDE MEDIATED NUCLEIC ACID RECOMBINATION” by Crameri et al., U.S. Pat. Nos. 6,319,714; 6,368,861; 6,376,246; 6,423,542; 6,426,224 and PCT/US00/01203; “USE OF CODON-VARIED OLIGONUCLEOTIDE SYNTHESIS FOR SYNTHETIC SHUFFLING” by Welch et al., U.S. Pat. No. 6,436,675; “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g. “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); “METHODS OF POPULATING DATA STRUCTURES FOR USE IN EVOLUTIONARY SIMULATIONS” by Selifonov and Stemmer, filed Jan. 18, 2000 (PCT/US00/01138); and “SINGLE-STRANDED NUCLEIC ACID TEMPLATE-MEDIATED RECOMBINATION AND NUCLEIC ACID FRAGMENT ISOLATION” by Affholter, filed Sep. 6, 2000 (U.S. Ser. No. 09/656,549); and U.S. Pat. Nos. 6,177,263; 6,153,410.

Non-Stochastic, or “Directed Evolution,” Methods

Exemplary protocols for generating variant sequences (e.g., modified sequences encoding chimeric polypeptides of the invention) in practicing the invention (e.g., making variants, such as non-immunogenic cannulae proteins for use in the drug delivery vehicles of the invention) include non-stochastic, or “directed evolution,” methods, such as, e.g., Gene Site Saturation Mutagenesis™ (GSSM™), synthetic ligation reassembly (SLR), or a combination thereof. These methods can be used to modify the nucleic acids to generate chimeric polypeptides with new or altered properties (e.g., chiral selection activity under high or low acidic or alkaline conditions, high or low temperatures, high or low salt conditions and the like; different substrate affinity; enantioselective activity; modified antibody binding activity, etc.). Polypeptides encoded by the modified nucleic acids can be screened for an activity before testing for proteolytic or other activity. Any testing modality or protocol can be used, e.g., using a capillary array platform. See, e.g., U.S. Pat. Nos. 6,361,974; 6,280,926; 5,939,250.

Saturation Mutagenesis, or, GSSM™

In one aspect of the invention, non-stochastic gene modification, a “directed evolution process,” is used to generate modified sequences encoding chimeric polypeptides of the invention or cannulae proteins for use in the drug delivery vehicles of the invention with new or altered properties. Variations of this method have been termed “Gene Site Saturation Mutagenesis™,” “site-saturation mutagenesis,” “saturation mutagenesis” or simply “GSSM™.” It can be used in combination with other mutagenization processes. See, e.g., U.S. Pat. Nos. 6,171,820; 6,238,884. In one aspect, GSSM comprises providing a template polynucleotide and a plurality of oligonucleotides, wherein each oligonucleotide comprises a sequence homologous to the template polynucleotide, thereby targeting a specific sequence of the template polynucleotide, and a sequence that is a variant of the homologous gene; generating progeny polynucleotides comprising non-stochastic sequence variations by replicating the template polynucleotide with the oligonucleotides, thereby generating polynucleotides comprising homologous gene sequence variations.

In one aspect, codon primers containing a degenerate N,N,G/T sequence are used to introduce point mutations into a polynucleotide, so as to generate a set of progeny polypeptides in which a full range of single amino acid substitutions is represented at each amino acid position, e.g., an amino acid residue in an enzyme active site or ligand binding site targeted to be modified. These oligonucleotides can comprise a contiguous first homologous sequence, a degenerate N,N,G/T sequence, and, optionally, a second homologous sequence. The downstream progeny translational products from the use of such oligonucleotides include all possible amino acid changes at each amino acid site along the polypeptide, because the degeneracy of the N,N,G/T sequence includes codons for all 20 amino acids. In one aspect, one such degenerate oligonucleotide (comprised of, e.g., one degenerate N,N,G/T cassette) is used for subjecting each original codon in a parental polynucleotide template to a full range of codon substitutions. In another aspect, at least two degenerate cassettes are used—either in the same oligonucleotide or not, for subjecting at least two original codons in a parental polynucleotide template to a full range of codon substitutions. For example, more than one N,N,G/T sequence can be contained in one oligonucleotide to introduce amino acid mutations at more than one site. This plurality of N,N,G/T sequences can be directly contiguous, or separated by one or more additional nucleotide sequence(s). In another aspect, oligonucleotides serviceable for introducing additions and deletions can be used either alone or in combination with the codons containing an N,N,G/T sequence, to introduce any combination or permutation of amino acid additions, deletions, and/or substitutions.

In one aspect, simultaneous mutagenesis of two or more contiguous amino acid positions is done using an oligonucleotide that contains contiguous N,N,G/T triplets, i.e. a degenerate (N,N,G/T)n sequence. In another aspect, degenerate cassettes having less degeneracy than the N,N,G/T sequence are used. For example, it may be desirable in some instances to use (e.g. in an oligonucleotide) a degenerate triplet sequence comprised of only one N, where said N can be in the first second or third position of the triplet. Any other bases including any combinations and permutations thereof can be used in the remaining two positions of the triplet. Alternatively, it may be desirable in some instances to use (e.g. in an oligo) a degenerate N,N,N triplet sequence.

In one aspect, use of degenerate triplets (e.g., N,N,G/T triplets) allows for systematic and easy generation of a full range of possible natural amino acids (for a total of 20 amino acids) into each and every amino acid position in a polypeptide (in alternative aspects, the methods also include generation of less than all possible substitutions per amino acid residue, or codon, position). For example, for a 100 amino acid polypeptide, 2000 distinct species (i.e. 20 possible amino acids per position X 100 amino acid positions) can be generated. Through the use of an oligonucleotide or set of oligonucleotides containing a degenerate N,N,G/T triplet, 32 individual sequences can code for all 20 possible natural amino acids. Thus, in a reaction vessel in which a parental polynucleotide sequence is subjected to saturation mutagenesis using at least one such oligonucleotide, there are generated 32 distinct progeny polynucleotides encoding 20 distinct polypeptides. In contrast, the use of a non-degenerate oligonucleotide in site-directed mutagenesis leads to only one progeny polypeptide product per reaction vessel. Nondegenerate oligonucleotides can optionally be used in combination with degenerate primers disclosed; for example, nondegenerate oligonucleotides can be used to generate specific point mutations in a working polynucleotide. This provides one means to generate specific silent point mutations, point mutations leading to corresponding amino acid changes, and point mutations that cause the generation of stop codons and the corresponding expression of polypeptide fragments.

In one aspect, each saturation mutagenesis reaction vessel contains polynucleotides encoding at least 20 progeny polypeptide molecules such that all 20 natural amino acids are represented at the one specific amino acid position corresponding to the codon position mutagenized in the parental polynucleotide (other aspects use less than all 20 natural combinations). The 32-fold degenerate progeny polypeptides generated from each saturation mutagenesis reaction vessel can be subjected to clonal amplification (e.g. cloned into a suitable host, e.g., E. coli host, using, e.g., an expression vector) and subjected to expression screening. When an individual progeny polypeptide is identified by screening to display a favorable change in property (when compared to the parental polypeptide, such as increased proteolytic activity under alkaline or acidic conditions), it can be sequenced to identify the correspondingly favorable amino acid substitution contained therein.

In one aspect, upon mutagenizing each and every amino acid position in a parental polypeptide using saturation mutagenesis as disclosed herein, favorable amino acid changes may be identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid, and each of two favorable changes) and 3 positions. Thus, there are 3×3×3 or 27 total possibilities, including 7 that were previously examined—6 single point mutations (i.e. 2 at each of three positions) and no change at any position.

In another aspect, site-saturation mutagenesis can be used together with another stochastic or non-stochastic means to vary sequence, e.g., synthetic ligation reassembly (see below), shuffling, chimerization, recombination and other mutagenizing processes and mutagenizing agents. This invention provides for the use of any mutagenizing process(es), including saturation mutagenesis, in an iterative manner.

Synthetic Ligation Reassembly (SLR)

In practicing the of the invention a non-stochastic gene modification system termed “synthetic ligation reassembly,” or simply “SLR,” a “directed evolution process,” can be used to generate modified sequences encoding chimeric polypeptides of the invention or cannulae proteins for use in the drug delivery vehicles of the invention with new or altered properties. SLR is a method of ligating oligonucleotide fragments together non-stochastically. This method differs from stochastic oligonucleotide shuffling in that the nucleic acid building blocks are not shuffled, concatenated or chimerized randomly, but rather are assembled non-stochastically. See, e.g., U.S. patent application Ser. No. 09/332,835 entitled “Synthetic Ligation Reassembly in Directed Evolution” and filed on Jun. 14, 1999 (“U.S. Ser. No. 09/332,835”). In one aspect, SLR comprises the following steps: (a) providing a template polynucleotide, wherein the template polynucleotide comprises sequence encoding a homologous gene; (b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides comprising homologous gene sequence variations.

SLR does not depend on the presence of high levels of homology between polynucleotides to be rearranged. Thus, this method can be used to non-stochastically generate libraries (or sets) of progeny molecules comprised of over 10100 different chimeras. SLR can be used to generate libraries comprised of over 101000 different progeny chimeras. Thus, aspects of the present invention include non-stochastic methods of producing a set of finalized chimeric nucleic acid molecule shaving an overall assembly order that is chosen by design. This method includes the steps of generating by design a plurality of specific nucleic acid building blocks having serviceable mutually compatible ligatable ends, and assembling these nucleic acid building blocks, such that a designed overall assembly order is achieved.

The mutually compatible ligatable ends of the nucleic acid building blocks to be assembled are considered to be “serviceable” for this type of ordered assembly if they enable the building blocks to be coupled in predetermined orders. Thus, the overall assembly order in which the nucleic acid building blocks can be coupled is specified by the design of the ligatable ends. If more than one assembly step is to be used, then the overall assembly order in which the nucleic acid building blocks can be coupled is also specified by the sequential order of the assembly step(s). In one aspect, the annealed building pieces are treated with an enzyme, such as a ligase (e.g. T4 DNA ligase), to achieve covalent bonding of the building pieces.

In one aspect, the design of the oligonucleotide building blocks is obtained by analyzing a set of progenitor nucleic acid sequence templates that serve as a basis for producing a progeny set of finalized chimeric polynucleotides. These parental oligonucleotide templates thus serve as a source of sequence information that aids in the design of the nucleic acid building blocks that are to be mutagenized, e.g., chimerized or shuffled. In one aspect of this method, the sequences of a plurality of parental nucleic acid templates are aligned in order to select one or more demarcation points. The demarcation points can be located at an area of homology, and are comprised of one or more nucleotides. These demarcation points are preferably shared by at least two of the progenitor templates. The demarcation points can thereby be used to delineate the boundaries of oligonucleotide building blocks to be generated in order to rearrange the parental polynucleotides. The demarcation points identified and selected in the progenitor molecules serve as potential chimerization points in the assembly of the final chimeric progeny molecules. A demarcation point can be an area of homology (comprised of at least one homologous nucleotide base) shared by at least two parental polynucleotide sequences. Alternatively, a demarcation point can be an area of homology that is shared by at least half of the parental polynucleotide sequences, or, it can be an area of homology that is shared by at least two thirds of the parental polynucleotide sequences. Even more preferably a serviceable demarcation points is an area of homology that is shared by at least three fourths of the parental polynucleotide sequences, or, it can be shared by at almost all of the parental polynucleotide sequences. In one aspect, a demarcation point is an area of homology that is shared by all of the parental polynucleotide sequences.

In one aspect, a ligation reassembly process is performed exhaustively in order to generate an exhaustive library of progeny chimeric polynucleotides. In other words, all possible ordered combinations of the nucleic acid building blocks are represented in the set of finalized chimeric nucleic acid molecules. At the same time, in another aspect, the assembly order (i.e. the order of assembly of each building block in the 5′ to 3 sequence of each finalized chimeric nucleic acid) in each combination is by design (or non-stochastic) as described above. Because of the non-stochastic nature of this invention, the possibility of unwanted side products is greatly reduced.

In another aspect, the ligation reassembly method is performed systematically. For example, the method is performed in order to generate a systematically compartmentalized library of progeny molecules, with compartments that can be screened systematically, e.g. one by one. In other words this invention provides that, through the selective and judicious use of specific nucleic acid building blocks, coupled with the selective and judicious use of sequentially stepped assembly reactions, a design can be achieved where specific sets of progeny products are made in each of several reaction vessels. This allows a systematic examination and screening procedure to be performed. Thus, these methods allow a potentially very large number of progeny molecules to be examined systematically in smaller groups. Because of its ability to perform chimerizations in a manner that is highly flexible yet exhaustive and systematic as well, particularly when there is a low level of homology among the progenitor molecules, these methods provide for the generation of a library (or set) comprised of a large number of progeny molecules. Because of the non-stochastic nature of the instant ligation reassembly invention, the progeny molecules generated preferably comprise a library of finalized chimeric nucleic acid molecules having an overall assembly order that is chosen by design. The saturation mutagenesis and optimized directed evolution methods also can be used to generate different progeny molecular species. It is appreciated that the invention provides freedom of choice and control regarding the selection of demarcation points, the size and number of the nucleic acid building blocks, and the size and design of the couplings. It is appreciated, furthermore, that the requirement for intermolecular homology is highly relaxed for the operability of this invention. In fact, demarcation points can even be chosen in areas of little or no intermolecular homology. For example, because of codon wobble, i.e. the degeneracy of codons, nucleotide substitutions can be introduced into nucleic acid building blocks without altering the amino acid originally encoded in the corresponding progenitor template. Alternatively, a codon can be altered such that the coding for an originally amino acid is altered. This invention provides that such substitutions can be introduced into the nucleic acid building block in order to increase the incidence of intermolecular homologous demarcation points and thus to allow an increased number of couplings to be achieved among the building blocks, which in turn allows a greater number of progeny chimeric molecules to be generated.

In another aspect, the synthetic nature of the step in which the building blocks are generated allows the design and introduction of nucleotides (e.g., one or more nucleotides, which may be, for example, codons or introns or regulatory sequences) that can later be optionally removed in an in vitro process (e.g. by mutagenesis) or in an in vivo process (e.g. by utilizing the gene splicing ability of a host organism). It is appreciated that in many instances the introduction of these nucleotides may also be desirable for many other reasons in addition to the potential benefit of creating a serviceable demarcation point.

In one aspect, a nucleic acid building block is used to introduce an intron. Thus, functional introns are introduced into a man-made gene manufactured according to the methods described herein. The artificially introduced intron(s) can be functional in a host cells for gene splicing much in the way that naturally-occurring introns serve functionally in gene splicing.

Optimized Directed Evolution System

In practicing the invention a non-stochastic gene modification system termed “optimized directed evolution system” can be used to generate modified sequences encoding chimeric polypeptides of the invention or cannulae proteins for use in the drug delivery vehicles of the invention with new or altered properties. Optimized directed evolution is directed to the use of repeated cycles of reductive reassortment, recombination and selection that allow for the directed molecular evolution of nucleic acids through recombination. Optimized directed evolution allows generation of a large population of evolved chimeric sequences, wherein the generated population is significantly enriched for sequences that have a predetermined number of crossover events.

A crossover event is a point in a chimeric sequence where a shift in sequence occurs from one parental variant to another parental variant. Such a point is normally at the juncture of where oligonucleotides from two parents are ligated together to form a single sequence. This method allows calculation of the correct concentrations of oligonucleotide sequences so that the final chimeric population of sequences is enriched for the chosen number of crossover events. This provides more control over choosing chimeric variants having a predetermined number of crossover events.

In addition, this method provides a convenient means for exploring a tremendous amount of the possible protein variant space in comparison to other systems. Previously, if one generated, for example, 10¹³ chimeric molecules during a reaction, it would be extremely difficult to test such a high number of chimeric variants for a particular activity. Moreover, a significant portion of the progeny population would have a very high number of crossover events which resulted in proteins that were less likely to have increased levels of a particular activity. By using these methods, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

One method for creating a chimeric progeny polynucleotide sequence is to create oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. Additional information can also be found, e.g., in U.S. Ser. No. 09/332,835; U.S. Pat. No. 6,361,974. The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. By utilizing these methods, one can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events. These methods are directed to the use of repeated cycles of reductive reassortment, recombination and selection that allow for the directed molecular evolution of a nucleic acid encoding a polypeptide through recombination. This system allows generation of a large population of evolved chimeric sequences, wherein the generated population is significantly enriched for sequences that have a predetermined number of crossover events. A crossover event is a point in a chimeric sequence where a shift in sequence occurs from one parental variant to another parental variant. Such a point is normally at the juncture of where oligonucleotides from two parents are ligated together to form a single sequence. The method allows calculation of the correct concentrations of oligonucleotide sequences so that the final chimeric population of sequences is enriched for the chosen number of crossover events. This provides more control over choosing chimeric variants having a predetermined number of crossover events.

In addition, these methods provide a convenient means for exploring a tremendous amount of the possible protein variant space in comparison to other systems. By using the methods described herein, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

In one aspect, the method creates a chimeric progeny polynucleotide sequence by creating oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. See also U.S. Ser. No. 09/332,835.

The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. One can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events.

Iterative Processes

In practicing the invention, these processes can be iteratively repeated. For example a nucleic acid (or, the nucleic acid) responsible for an altered phenotype of a chimeric polypeptide of the invention is identified, re-isolated, again modified, re-tested for activity using the methods of the invention. This process can be iteratively repeated until a desired phenotype is engineered. For example, an entire biochemical anabolic or catabolic pathway can be engineered into a cell, including proteolytic activity.

Similarly, if it is determined that a particular oligonucleotide has no affect at all on the desired trait, it can be removed as a variable by synthesizing larger parental oligonucleotides that include the sequence to be removed. Since incorporating the sequence within a larger sequence prevents any crossover events, there will no longer be any variation of this sequence in the progeny polynucleotides. This iterative practice of determining which oligonucleotides are most related to the desired trait, and which are unrelated, allows more efficient exploration all of the possible protein variants that might be provide a particular trait or activity.

Producing Sequence Variants

In practicing the methods of the invention nucleic acid variants can be generated using genetic engineering techniques such as site directed mutagenesis, random chemical mutagenesis, Exonuclease III deletion procedures, and standard cloning techniques. Alternatively, such variants, fragments, analogs, or derivatives may be created using chemical synthesis or modification procedures. Other methods of making variants are also familiar to those skilled in the art. These include procedures in which nucleic acid sequences obtained from natural isolates are modified to generate nucleic acids which encode polypeptides having characteristics which enhance their value in industrial or laboratory applications. In such procedures, a large number of variant sequences having one or more nucleotide differences with respect to the sequence obtained from the natural isolate are generated and characterized. These nucleotide differences can result in amino acid changes with respect to the polypeptides encoded by the nucleic acids from the natural isolates.

For example, variants may be created using error prone PCR. In error prone PCR, PCR is performed under conditions where the copying fidelity of the DNA polymerase is low, such that a high rate of point mutations is obtained along the entire length of the PCR product. Error prone PCR is described, e.g., in Leung, D. W., et al., Technique, 1:11-15, 1989) and Caldwell, R. C. & Joyce G. F., PCR Methods Applic., 2:28-33, 1992. Briefly, in such procedures, nucleic acids to be mutagenized are mixed with PCR primers, reaction buffer, MgCl₂, MnCl₂, Taq polymerase and an appropriate concentration of dNTPs for achieving a high rate of point mutation along the entire length of the PCR product. For example, the reaction may be performed using 20 fmoles of nucleic acid to be mutagenized, 30 pmole of each PCR primer, a reaction buffer comprising 50 mM KCl, 10 mM TRIS® (hydroxymethyl amino methane hydrochloride) (pH 8.3) and 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR may be performed for 30 cycles of 94° C. for 1 min, 45° C. for 1 min, and 72° C. for 1 min. However, it will be appreciated that these parameters may be varied as appropriate. The mutagenized nucleic acids are cloned into an appropriate vector and the activities of the polypeptides encoded by the mutagenized nucleic acids is evaluated.

Variants may also be created using oligonucleotide directed mutagenesis to generate site-specific mutations in any cloned DNA of interest. Oligonucleotide mutagenesis is described, e.g., in Reidhaar-Olson (1988) Science 241:53-57. Briefly, in such procedures a plurality of double stranded oligonucleotides bearing one or more mutations to be introduced into the cloned DNA are synthesized and inserted into the cloned DNA to be mutagenized. Clones containing the mutagenized DNA are recovered and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, e.g., U.S. Pat. No. 5,965,408.

Still another method of generating variants is sexual PCR mutagenesis. In sexual PCR mutagenesis, forced homologous recombination occurs between DNA molecules of different but highly related DNA sequence in vitro, as a result of random fragmentation of the DNA molecule based on sequence homology, followed by fixation of the crossover by primer extension in a PCR reaction. Sexual PCR mutagenesis is described, e.g., in Stemmer (1994) Proc. Natl. Acad. Sci. USA 91:10747-10751. Briefly, in such procedures a plurality of nucleic acids to be recombined are digested with DNase to generate fragments having an average size of 50-200 nucleotides. Fragments of the desired average size are purified and resuspended in a PCR mixture. PCR is conducted under conditions which facilitate recombination between the nucleic acid fragments. For example, PCR may be performed by resuspending the purified fragments at a concentration of 10-30 ng/:1 in a solution of 0.2 mM of each dNTP, 2.2 mM MgCl₂, 50 mM KCL, 10 mM TRIS® (hydroxymethyl amino methane hydrochloride), pH 9.0, and 0.1% TritonX-100. 2.5 units of Taq polymerase per 100:1 of reaction mixture is added and PCR is performed using the following regime: 94° C. for 60 seconds, 94° C. for 30 seconds, 50-55° C. for 30 seconds, 72° C. for 30 seconds (30-45 times) and 72° C. for 5 minutes. However, it will be appreciated that these parameters may be varied as appropriate. In some aspects, oligonucleotides may be included in the PCR reactions. In other aspects, the Klenow fragment of DNA polymerase I may be used in a first set of PCR reactions and Taq polymerase may be used in a subsequent set of PCR reactions. Recombinant sequences are isolated and the activities of the polypeptides they encode are assessed.

Variants may also be created by in vivo mutagenesis. In some aspects, random mutations in a sequence of interest are generated by propagating the sequence of interest in a bacterial strain, such as an E. coli strain, which carries mutations in one or more of the DNA repair pathways. Such “mutator” strains have a higher random mutation rate than that of a wild-type parent. Propagating the DNA in one of these strains will eventually generate random mutations within the DNA. Mutator strains suitable for use for in vivo mutagenesis are described, e.g., in PCT Publication No. WO 91/16427.

Variants may also be generated using cassette mutagenesis. In cassette mutagenesis a small region of a double stranded DNA molecule is replaced with a synthetic oligonucleotide “cassette” that differs from the native sequence. The oligonucleotide often contains completely and/or partially randomized native sequence.

Recursive ensemble mutagenesis may also be used to generate variants. Recursive ensemble mutagenesis is an algorithm for protein engineering (protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described, e.g., in Arkin (1992) Proc. Natl. Acad. Sci. USA 89:7811-7815.

In some aspects, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described, e.g., in Delegrave (1993) Biotechnology Res. 11:1548-1552. Random and site-directed mutagenesis are described, e.g., in Arnold (1993) Current Opinion in Biotechnology 4:450-455.

In some aspects, the variants are created using shuffling procedures wherein portions of a plurality of nucleic acids which encode distinct polypeptides are fused together to create chimeric nucleic acid sequences which encode chimeric polypeptides as described in, e.g., U.S. Pat. Nos. 5,965,408; 5,939,250.

Optimizing Codons to Achieve High Levels of Protein Expression in Host Cells

In one aspect of the invention, nucleic acids are mutated to modify codon usage.

In one aspect, methods of the invention comprise modifying codons in a nucleic acid encoding a modified sequence encoding a chimeric polypeptide of the invention to increase or decrease its expression in a host cell, e.g., a bacterial, insect, mammalian, yeast or plant cell. The method can comprise identifying a “non-preferred” or a “less preferred” codon in protein-encoding nucleic acid and replacing one or more of these non-preferred or less preferred codons with a “preferred codon” encoding the same amino acid as the replaced codon and at least one non-preferred or less preferred codon in the nucleic acid has been replaced by a preferred codon encoding the same amino acid. A preferred codon is a codon over-represented in coding sequences in genes in the host cell and a non-preferred or less preferred codon is a codon under-represented in coding sequences in genes in the host cell.

Methodologies and Devices

In practicing the invention, a variety of apparatus and methodologies can be used, e.g., using the chimeric monomers and polymers for chiral selection, to determine the efficiency of the chiral separation from a racemic mixture, as biosynthetic pathways, as selection scaffoldings, to screen for variant chimeric polypeptides, to determine the extent of nanotubule formation, and the like.

Capillary Arrays

Capillary arrays, such as the GIGAMATRIX™, Diversa Corporation, San Diego, Calif., can be used to practice the invention. Nucleic acids or polypeptides (the chimeric monomers and polymers of the invention) or other compositions (e.g., substrates or co-factors for using the nanotubule biosynthetic pathways of the invention, antibodies or other compounds for binding to chimeric monomers of the invention) can be immobilized to or applied to an array, including capillary arrays. Arrays can be used in the chiral selection methods of the invention. Capillary arrays can provide a system for holding and screening samples, monomers of the invention, chiral products selected by the methods of the invention, substrates and co-factors and products used in the biosynthetic pathway methods of the invention, and the like.

A sample apparatus can include a plurality of capillaries formed into an array of adjacent capillaries, wherein each capillary comprises at least one wall defining a lumen for retaining a sample. The apparatus can further include interstitial material disposed between adjacent capillaries in the array, and one or more reference indicia formed within of the interstitial material. A capillary for screening a sample, wherein the capillary is adapted for being bound in an array of capillaries, can include a first wall defining a lumen for retaining the sample, and a second wall formed of a filtering material, for filtering excitation energy provided to the lumen to excite the sample.

A polypeptide or other composition can be introduced into a first component into at least a portion of a capillary of a capillary array. Each capillary of the capillary array can comprise at least one wall defining a lumen for retaining the first component. An air bubble can be introduced into the capillary behind the first component. A second component can be introduced into the capillary, wherein the second component is separated from the first component by the air bubble. A sample of interest can be introduced as a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall. The method can further include removing the first liquid from the capillary tube, wherein the bound detectable particle is maintained within the capillary, and introducing a second liquid into the capillary tube.

The capillary array can include a plurality of individual capillaries comprising at least one outer wall defining a lumen. The outer wall of the capillary can be one or more walls fused together. Similarly, the wall can define a lumen that is cylindrical, square, hexagonal or any other geometric shape so long as the walls form a lumen for retention of a liquid or sample. The capillaries of the capillary array can be held together in close proximity to form a planar structure. The capillaries can be bound together, by being fused (e.g., where the capillaries are made of glass), glued, bonded, or clamped side-by-side. The capillary array can be formed of any number of individual capillaries, for example, a range from 100 to 4,000,000 capillaries. A capillary array can form a micro titer plate having about 100,000 or more individual capillaries bound together.

Arrays, or “Biochips”

In practicing the invention polypeptides of the invention can be immobilized to or applied to an array. Arrays can be used to practice the methods of the invention, e.g., chiral selection from a racemic mixture. Polypeptide arrays” can be used to simultaneously quantify or select for a plurality of proteins. The present invention can be practiced with any known “array,” also referred to as a “microarray” or “DNA array” or “nucleic acid array” or “polypeptide array” or “antibody array” or “biochip,” or variation thereof. Arrays are generically a plurality of “spots” or “target elements,” each target element comprising a defined amount of one or more biological molecules immobilized onto a defined area of a substrate surface for specific binding to a sample molecule. Any immobilization method can be used, e.g., immobilization upon an inert support such as diethylaminoethyl-cellulose, porous glass, chitin or cells. In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

The polymer of the present invention may also be used in various so-called biochip applications. The polymer may be arrayed, on its end, on silicon or aluminum wafers for use as a scaffold to anchor proteins in a high-density, three-dimensional format for protein-protein interaction screening applications. Such an arrayed polymer may be valuable in research to identify and validate novel drug target molecules. Some biochip applications using known probes have been disclosed in U.S. Pat. Nos. 6,174,683 and 6,242,246.

In one embodiment, in order to provide a three-dimensional gel matrix useful in producing a biochip, the polymer chosen to form the gel matrix must have a number of desirable properties. These properties include, for example: 1) adequate pore size and high water content to permit diffusion of molecules in and out of the matrix; 2) the ability to bind to the surface of a substrate, such as glass; 3) sufficient transparency, in its fully polymerized state, to reduce optical interference with fluorescent tags; and 4) sufficient structural integrity, when fully polymerized, to withstand the forces encountered during use. Furthermore, the selected gel is preferably easy to produce and use.

Hydrogels are a class of polymers that meet with these criteria. Hydrogels are hydrophilic network polymers, which are glassy in the dehydrated state and swollen in the presence of water to form an elastic gel. The polyacrylamide gel matrices described in Ershov, et al., are hydrogels having a water content, at equilibrium, of about 95% to 97%, providing favorable diffuseability for target molecules such as DNA's. See for example, U.S. Pat. Nos. 5,741,700, 5,770,721 and 5,756,050, issued to Ershov, et al., on Apr. 21, 1998, Jun. 23, 1998 and May 26, 1998, respectively and U.S. Pat. No. 5,552,270, issued to Khrapko, et al., issued Sep. 3, 1996.

In addition to the polyacrylamide gel system of Ershov, et al., polyurethane-based hydrogel polymers are well known and have been used extensively in the production of absorbent materials such as surgical dressings, diapers, bed pads, catamenials, and the like. The polyurethane-based hydrogels used in these materials advantageously absorb large quantities of liquid quickly and in a relatively uniform manner such that the basic overall shape of the gel material is maintained. Further, the moisture absorbed by these materials is retained in the absorbent material even under an applied pressure. Such polyurethane-based hydrogels are described, for example, in U.S. Pat. No. 3,939,123, issued to Mathews, et al., Feb. 17, 1976 and U.S. Pat. No. 4,110,286, issued to Vandegaer, et al., Aug. 29, 1978.

In one embodiment, the biochip of the present invention uses a hydrogel based on a self-assembling polymer in accordance with the present invention. Alternatively, a the hydrogel may be based on a prepolymer of polyethyleneoxide, or a copolymer of polyethyleneoxide and polypropyleneoxide, capped with water-active diisocyanates and lightly cross-linked with polyols such that the quantity of isocyanates present is predictable for example is at most about 0.8 meq/g. Frequently used diisocyanates include aromatic-based diisocyanates, such as toluene diisocyanate or methylene diphenyl-isocyanate, as well as aliphatic diisocyanates, such as isophorone diisocyanate. The polymerization of the prepolymer, which may be preformulated in water-miscible organic solvent, takes place simply by the addition of water. One advantage of the water-activated polymerization and/or the self-assembly polymerization methods of the present invention is that they allow for derivatization of the pre-polymer with an appropriate biomolecular probe prior to or simultaneously with polymerization.

In another embodiment, the self-assembled polymer of the present invention may be attached to the hydrogel to provide, for example, a three-dimensional structural network for the biochip. Attachment to the hydrogel may also be used for other purposes such as self-assembly of complex components of the chip, to provide structural integrity, etc.

In another embodiment, prior to polymerization, the hydrogel is derivatized with a biomolecule such as a probe of the present invention as described above, in an organic solvent using a simple two to three-minute reaction between the probe, preferably peptides or nucleic acids which have been previously derivatized with amine, and the isocyanates of the prepolymer. In order to prevent premature polymerization of the hydrogel in the present embodiment, the derivatization reaction is carried out in aprotic water-miscible organic solvent such as, for example, dimethylformamide (DMF), N-methyl-2-pyrrolidinone (NMP), acetone, acetonitrile or others. Thus, prior to swelling of the hydrogel or dispensing of the hydrogel onto the substrate, biomolecular probes are covalently bound to the polyurethane-based prepolymer gel. Following such derivatization, the addition of water initiates polymerization, resulting in biomolecular-derivatized hydrogels, for example, PNA-derivatized hydrogels.

In this embodiment, the use and presence of aprotic solvent in the derivatization of the hydrogel serves at least four purposes. First, it helps generate a homogeneous solution of the prepolymer in water. Second, it serves to separate the derivatization step from the polymerization step, whereby almost quantitative yield of biomolecule derivatization to the hydrogel can be achieved. Third, it serves to slow down the generation of carbon dioxide during the polymerization step and effervesce carbon dioxide efficiently by lowering the viscosity of the polymerizing mixture. In the polymerization of the polyurethane-based hydrogels preferred herein, carbon dioxide is generated by the reaction of water with the isocyanate groups of the hydrogel prepolymer. Controlling the generation of carbon dioxide and its escape from the gel are critical to providing an effective, useful biochip. If the polymerization occurs too quickly and in a highly viscous mixture, the carbon dioxide generated thereby is not able to escape and becomes trapped within the gel resulting in a discrete foam matrix. While such is not a problem when polyurethane-based hydrogels are used in diapers, bed pads or similar known uses, continuum of the gel matrix is critical in its use in biochips in order to permit accurate and efficient detection of fluorescence indicative of successful hybridization.

A fourth and final advantage to the use of an aprotic solvent to derivatize the hydrogel in the present embodiment is that its presence enhances the optical transparency of the hydrogel by reducing precipitation of the prepolymer. The ratio of aprotic solvent to water must be higher than about 0.25 to allow sufficiently slow polymerization of the gel and, therefore, slow generation of CO₂, to result in a continuous and transparent gel matrix, in accordance with the present invention. The total time required for derivatization and polymerization of the hydrogel is most preferably about thirty minutes. This is in stark contrast to the twenty-four to forty-eight hours required for preparation of polyacrylamide based biochips. Furthermore, the quantity of biomolecule such as the probe, for example bound to the prepolymer may easily be adjusted by simply varying the amount of biomolecule added to the reaction (for example, where probe is the biomolecule to be bound to the gel, from about 10 fmol up to about 1 pmol of probes may be used), thereby permitting greater control over the concentration of capture probes within each hydrogel microdroplet.

In this embodiment, the hydrogel is derivatized with the probe then deposited onto the solid substrate, after initiation and before completion of polymerization thereof. This may be accomplished by any convenient method, for example by use of a microspotting machine. The gel is preferably deposited to form an array of microdroplets. It will be appreciated by those of skill in the art that the substrate surface will generally have to be derivatized prior to addition of the hydrogel, for example, in preferred embodiments, where glass is used as the substrate, the glass is derivatized with amine prior to deposit of the polymerizing hydrogel onto its surface. Thus, the polymerizing hydrogel, derivatized with a biomolecular capture probes such as DNAs, is able to bind to the substrate as it is deposited onto the derivatized glass substrate, via reaction of active isocyanate groups within the prepolymer with the amines located on the surface of the glass thereby providing covalent attachment of the hydrogel to the substrate. Most advantageously, all reactions involved in this system, namely (1) the derivatization of hydrogel prepolymer with the biomolecular probe, (2) the polymerization of hydrogel and (3) the binding of derivatized hydrogel to the substrate surface, involve the formation of strong urea bonds. These provide mechanical integrity to the microdroplet array, and significantly increase the half-life of the biochip as compared with the polyacrylamide-based biochip described in the prior art.

In other embodiments described herein, the hydrogel droplets, once polymerized on the substrate, are at least about 30 μm thick, more preferably at least about 50 μm thick and most preferably between about 50 μm and 100 μm thick. Furthermore, the droplets will be generally elliptical in shape, as opposed to the square gel cells previously known. It will be readily appreciated that the larger size of the gel droplets (or cells) of the present invention permit a significant increase in the quantity of biomolecular probe immobilized therein, thereby increasing the sensitivity of the biochip and facilitating its use.

In alternative embodiments contemplated herein, water soluble biomolecules, such as the probe of the present invention, DNA or other oligonucleotides, are bound to the hydrogel instead of the organic soluble biomolecules previously described. In these embodiments, it is not possible to first derivatize the hydrogel prepolymer and then initiate polymerization. However, the polyurethane-based hydrogels may be derivatized and polymerized in a single reaction and that such reaction may be adequately controlled to provide a derivatized hydrogel having a relatively predictable quantity of water soluble biomolecular probe attached thereto. In particular, in these embodiments, the hydrogel prepolymer is first dissolved in an organic solvent. The DNA or other water-soluble biomolecule, in aqueous buffer solution, is then added to the prepolymer in a quantity and under appropriate conditions such that the hydrogel is both derivatized with the biomolecular probe and is polymerized. As the hydrogel is polymerizing and before the polymerization is complete, it may be microspotted onto a suitable substrate, as previously described.

Alternatively, the polymer of the present invention may be arrayed in a similar manner as described above, but for the purpose of acting as a molecular sieve. In this embodiment, the arrayed polymer may be used to separate nucleic acid samples as the nucleic acid samples pass through a matrix of the arrayed polymers. Such arrayed polymers may be used in high throughput DNA sequencing or SNP analyses.

Nanorobots and Biomedical Applications and Devices

The compositions of the invention, e.g., the polymer of the invention, may be used as (or used in) molecular machine components such as shafts or gears, for nanorobots for a wide variety of applications, including biomedical applications. Additionally, the polymer of the present invention may be used as support struts for various structures, or as nanoscopic screws for attachment of tissues during highly intricate surgical procedures. For example, the size of the polymer of the present invention may be controlled through the polymerization conditions and, therefore, the length of the polymer rod may be properly controlled to achieve a desired length. The end units of the polymer (rod) may be varied through using different end capping units. Such a custom designed polymer may be then used as a component in molecular machine or nanomachine.

Attaching one or more enzymes, which catalyze synthesis in a pathway, to one or more of the monomeric polypeptide units in the polymer of the present invention may provide a high-density immobilized, stable, economical biocatalyst for high value chemicals and pharmaceuticals. This type of immobilized biocatalyst may be removed and recycled or destroyed in a controlled way using simple chemical or enzymatic proteolysis.

In addition, the polymer may be used as a universal chiral separating agent based upon the principle of differential interaction of D- and L-isomers with the underlying, L-chiral monomeric polypeptide units contained in the polymer. For example, the polymer of the present invention may be packed or co-packed with a filler into an HPLC column to be used as a chiral HPLC column. Alternatively, the polymer may be immobilized on a substrate such as a cross-linked polystyrene substrate so that the immobilized polymer may be used a chiral separation medium. Depending on the degree of polymerization and the resulting molecular size of the polymer, DNA/RNA/Protein purification resins with different filtration properties may be produced. In a preferred embodiment, the polymer may be used as a separating agent for high value pharmaceutical compounds, which often require not only high chemical purity but also high enantiomatic purity, e.g. containing predominantly one of the enantiomers.

In one embodiment of the method of using the polymer as a separation agent according to the present invention, the polymer may modified by introducing an unsaturated side chain such as a styrene moiety using common synthetic methods such glycosylation using a styrene substituted glycoside. Thereafter, the modified polymer may be copolymerized with styrene and divinylbenzene using emulsion or suspension polymerization methods to form a universal chiral separation resin with the polymer covalently attached to the resin. Alternatively, the styrene and divinylbenzene may be copolymerized in the presence of an unmodified polymer of the present invention to form a resin with the polymer being non-covalently attached. The resin is then packed into an HPLC column and the packed column is installed in a HPLC system to be used to separate pharmaceutical compounds.

In one aspect, a chimeric polypeptide or a polymer of the present invention may be used a lubricant due to its high thermal stability. For example, the polymer of the present invention may be used as a lubricant either alone or mixed with another known lubricant. This type of lubricant may achieve an improved lubrication efficiency and a wider operating temperature range. Typical lubricants have a relatively narrow operating temperature range because at high temperatures, the viscosity of the typical lubricant tends to be too low to achieve a good lubrication efficiency. On the other hand, at a low temperatures, the typical lubricant may be too viscous to achieve a good lubrication efficiency. However, the polymer of the present invention has a unique molecular shape (rod like), therefore its viscosity vs. temperature profile is much flatter than the typical hydrocarbon lubricant. In a preferred embodiment, the polymer of the present invention may be dissolved in water or other suitable solvent form a lubricant. The concentration of the polymer may be optimized based on the desired operating temperature and molecular weight of the polymer.

In one aspect, a chimeric polypeptide or polymer of the present invention may also be used in uniform coating of paint due to its consistent structure. Normally, the conventional coating requires a filler such as TiO₂ for both cosmetic and durability purposes. Recently, coatings have been formulated with plastic fillers. However, fillers tend to have one common problem, which is their irregular shape, which makes it difficult to control the rheology of the formulated coating. In contrast, the polymer of the present invention may have a well defined and controlled shape and size. Therefore, the polymer of the present invention may be used as a filler in coating formulations. In addition, the polymer of the present invention may be produced using a biotechnology process such as fermentation. In a preferred embodiment, the coating composition of the present invention may include a uniform blend of one or more polymeric binders dispersed in a liquid medium, which liquid medium consists essentially of at least one component selected from the group consisting of water and organic solvents and a filler, wherein the filler comprises a polymer made by self-assembly of a plurality of polypeptides, wherein each of the plurality of polypeptides has at least 50% homology to a polypeptide having a sequence selected from the group consisting of SEQ ID NOS: 2, 4, 6, 8 and 10.

In one aspect, a chimeric polypeptide or polymer of the present invention may be used in place of conventional polymers produced from petrochemicals to produce fibers, plastics and resins. The polymer of the present invention has many advantages over such polymers. For example, the polymer of the present invention has a regular structure. Therefore, one can tailor the properties of the final product of the polymer by controlling the regular structure. Furthermore, the polymer of the present invention may be made from renewable resources. In addition, because of its regular structure, the polymer of the present invention may have some properties such as forming liquid crystals, which allow the strength of the polymer may be increased dramatically.

By incorporating a charged group at one end of the polymer of the present invention, the polymer may align to an electric field. Such aligned polymers would polarize light. By alternating the field applied to the aligned polymers, an optical switch may be produced. There are many applications for such optical switches such as Spatial Light Modulators, “Liquid Crystal” type displays, and optical switches for communications. The methods of forming liquid crystals using the polymer of the present invention are known to a skilled person in the art. In addition, the polymer of the present invention may be used in an optical waveguide. An optical waveguide for processing a beam of light of the present invention includes an elongated body of a light transmitting medium containing one or more liquid crystals therein, the body having first and second sides and entry and exit end faces that extend between the first and second sides, the beam of light entering the body through the entry end face and exiting the body through the exit end face after traveling through the body along a path between the entry and exit end faces; and a first electrode and a second electrode on the first and second sides of the body respectively for establishing an electric field between the first and second sides of the body, wherein said one or more liquid crystals comprises a chimeric polypeptide or polymer of the invention made by self-assembly of a plurality of cannulae polypeptides, e.g., polypeptides having amino acid sequences as in SEQ ID NO:2, SEQ ID NO:4, SEQ ID NO:6, and sequences substantially identical thereto.

In another aspect, the invention provides a method of producing a heat stable enzyme. In the method, a first known enzyme may be fused or connected with a cannulae polypeptide of the invention, including chimeric proteins of the invention comprising polypeptides having amino acid sequences as in SEQ ID NO:2, SEQ ID NO:4, SEQ ID NO:6, and sequences substantially identical thereto, to form a third protein or polypeptide having an improved thermal stability in comparison with the first known enzyme by itself. The formed third protein or polypeptide generally contains both the amino acid sequence of the first known enzyme and the second amino acid sequence comprising proteins of the invention and may at least partially retain the enzymatic activities of the first known enzyme. The formed third protein or polypeptide may be further polymerized to form a polymer containing a plurality of the formed third proteins or polypeptides and still at least partially retaining the enzymatic activities of the first known enzyme. The fusion or connecting of the first known enzyme with the second amino acid sequence may be carried out using a chemical method such as reacting the N-terminal of one molecule with the C-terminal of another molecule. The fusion may be carried out by fusing a first gene encoding the first enzyme and a second gene encoding the second amino acid sequence together to form a third gene encoding both using standard molecular cloning techniques. The third gene is then cloned into an appropriate over-expression vector and is expressed in suitable host cells or organisms to produce the third protein or polypeptide. Once expressed, the third protein or polypeptide may be purified from the host cells, organisms or proteins by heat treatment to denature the heat-labile host proteins contained in the host cells. Exemplary denaturing conditions are 80° C.-100° C. for 2-20 minutes. The heat-stable third protein or polypeptide is further purified from other contaminating proteins by conventional ion exchange chromatography. The purified third protein or polypeptide may be further polymerized into a polymer by heating a solution containing the third proteins or polypeptides to 80° C. in the presence of millimolar calcium and magnesium cations. The formed polymer may be isolated by centrifugation at 30,000 g for 30 minutes.

This process is further illustrated in FIG. 6. Amino acid sequence 41 is a cannulae amino acid sequence, e.g., SEQ ID NO:2, SEQ ID NO:4, SEQ ID NO:6, and sequences substantially identical thereto (including chimeric proteins of the invention). Enzyme 43 is an enzyme having a particular enzymatic activity and may be heat labile. Amino acid sequence 41 and enzyme 43 are fused together using a suitable method to form a protein 45, which not only retains at least some of the particular enzymatic activity but also is more thermally stable than enzyme 43.

These fused enzymes or proteins are generally more thermally stable than typical conventional enzymes and, therefore, can be used in applications requiring high operating temperatures. These fused enzymes or proteins, and polymers self-assembled therefrom, may retain one or more of the enzymatic activities of the original unfused enzymes.

Antibodies

The compositions and methods of the invention can be practice using antibodies. For example, a heterologous polypeptide of a chimeric protein of the invention can be an antibody, e.g., a catalytic antibody for use in a biosynthetic pathway of the invention, or, an antibody that specifically binds to an enzyme, co-factor, substrate and the like for use in a biosynthetic pathway of the invention, or, an antibody that binds to a chiral selection protein or peptide used in the methods of the invention. Antibodies also can be used in immunoprecipitation, staining, immunoaffinity columns, and the like, to, e.g., purify chiral selection products or products of the biosynthetic pathways of the invention.

Methods of doing assays, e.g., ELISAs, with polyclonal and monoclonal antibodies are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

The ability of proteins in a biological sample to bind to the antibody may be determined using any of a variety of procedures familiar to those skilled in the art. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. Particular assays include ELISA assays, sandwich assays, radioimmunoassays, and Western Blots.

Kits

The invention provides kits comprising materials for practicing the invention, including monomers and polymers, e.g., nanotubules, of the invention. The kits also can contain instructional material teaching the methodologies and uses of the invention, as described herein.

The invention will be further described with reference to the following examples; however, it is to be understood that the invention is not limited to such examples.

### EXAMPLES

TABLE 3
Chemicals Used In The Following Examples

| Substance | Source |
|---|---|
| α-³³P-dCTP | NEN, Dreieich |
| α-³⁵S-dATP | NEN, Dreieich |
| Acrylamide (reinst) | Serva, Heidelberg |
| Agar | Oxoid, Basingstoke (England) |
| Agarose | Roth, Karlsruhe |
| Agarose low melt | Roth, Karlsruhe |
| Agarose Seakem | Biozym, Hess. Odendorf |
| Ammonium sulfate | Sigma, Deisenhofen |
| Ampicillin | USB, Braunschweig |
| BCIP | Boehringer, Mannheim |
| 2-mercapto-ethanol | Roth, Karlsruhe |
| Bis-Tris | USB, Braunschweig |
| Blocking reagents | Boehringer, Mannheim |
| Bromophenol blue | Serva, Heidelberg |
| Caps | Sigma, Deisenhofen |
| Cesium chloride | Roth, Mannheim |
| CDP-Star ™ chemiluminescence substrate | Boehringer, Mannheim |
| Chloramphenicol | USB, Braunschweig |
| Coomassie brilliant blue R250 | Serva, Heidelberg |
| DEPC | Serva, Heidelberg |
| DIG DNA labeling mixture (10×) | Boehringer, Mannheim |
| DIG Easy Hyb | Boehringer, Mannheim |
| DIG-11-dUTP | Boehringer, Mannheim |
| Didesoxy nucleotides | Boehringer, Mannheim |
| DTT | Serva, Heidelberg |
| EDTA | Serva, Heidelberg |
| Ethanol (97%-99%) | Roth, Karlsruhe |
| Ethidium bromide | Sigma, Deisenhofen |
| Gases and gaseous mixtures | Linde, Munich |
| Glutathione (ox.) | Sigma, Deisenhofen |
| Guanidine hydrochloride | ICN, Eschwege |
| Guanidinium thiocyanate | Sigma, Deisenhofen |
| Yeast extract | Difco, Detroit (USA) |
| IPTG | Boehringer, Mannheim |
| Isoamyl alcohol (3-methyl-1-butanol) | Fluka, Neu-Ulm |
| Iodacetamide | Sigma, Deisenhofen |
| Binding matrix | Sigma, Deisenhofen |
| L-arginine | Aldrich, Steinheim |
| Lauroyl sarcosine | Sigma, Deisenhofen |
| L-cystine | Sigma, Deisenhofen |
| Malachite green hydrochloride | Sigma, Deisenhofen |
| MES | USB, Braunschweig |
| Sodium thiosulfate | Riedel-de-Haën, Seelze |
| NBT | Boehringer, Mannheim |
| N,N-methylene bisacrylamide (2×) | Serva, Heidelberg |
| Nonidet NP 40 | Sigma, Deisenhofen |
| Okadaic acid | ICN, Eschwege |
| Phenol (buffer saturated, Tris ® (pH 8.0) | Appligene, Heidelberg |
| ³²Pᵢ | Amersham, Braunschweig |
| Ponceau S | Serva, Heidelberg |
| Resazurin | Serva, Heidelberg |
| Rubidium chloride | Sigma, Deisenhofen |
| SDS | Serva, Heidelberg |
| Silicone solution | Serva, Heidelberg |
| Spermidine | Serva, Heidelberg |
| TEMED | Sigma, Deisenhofen |
| Trichloroacetic acid | Riedel-de-Haën, Seelze |
| Tricine | Sigma, Deisenhofen |
| Tris ® | USB, Braunschweig |
| Triton X-100 | Sigma, Deisenhofen |
| Trypton | Difco, Detroit (USA) |
| Tween 20 | Sigma, Deisenhofen |
| X-gal | AGS, Heidelberg |

All other chemicals were obtained from Merck, Darmstadt.

Unless stated otherwise, all substances were of purity grade p.A.

TABLE 4
Enzymes Used In the Following Examples

| Enzyme | Company |
|---|---|
| β-agarase (1 U/μl) | New England Biolabs, |
| | Schwalbach |
| Alkaline phosphatase | Promega, Heidelberg |
| (calf intestine) (5 U/μl) | |
| Ampli-Taq-DNA polymerase (5 U/μl) | Perkin Elmer, Norwalk (USA) |
| Klenow fragment (2 U/μl) | Boehringer, Mannheim |
| Pfu-DNA polymerase (2.5 U/μl) | Stratagene, Heidelberg |
| Proteinase K | Boehringer, Mannheim |
| Restriction enzymes | Boehringer, Mannheim, and |
| | New England Biolabs, |
| | Schwalbach |
| RNase, DNase-free (0.5 mg/ml) | Boehringer, Mannheim |
| RNasin ® (40 U/μl) | Promega, Mannheim |
| Subtilisin | Boehringer, Mannheim |
| T4-DNA ligase (1 U/μl) | Boehringer, Mannheim |

TABLE 5
Organisms Used In The Following Examples

| Organism | Reference |
|---|---|
| Pyrodictium abyssi | Deininger W., 1994 |
| isolate TAG11 | |
| Hyperthermus butylicus | Zillig et al., 1990; DSMZ 5456 |
| E. coli DH5α | Woodcock et al., 1989; |
| | [Stratagene, Heidelberg] |
| E. coli Y1090 | Young and Davis, 1983; |
| | [Stratagene, Heidelberg] |
| E. coli BL 21 (DE3) | Phillips et al., 1984; |
| | [Stratagene, Heidelberg] |

Other representatives of archaea, which were used for the study of genetic propagation of the cannulae genes, originate from the culture collection of the Regensburg Archaeal Center.

TABLE 6
Oligonucleotides Used In The Following Examples

| | | Position |
|---|---|---|
| Label | Sequence (5′ − >3′) | (canA) |
| M13 forward | GCCAGGGTTTTCCCAGTCACGA | — |
| M13 reverse | AGCGGATAACAATTTCACACAGG | — |
| T3 promoter | ATTAACCCTCACTAAAG | — |
| T7 promoter | TAATACGACTCACTATAGGGG | — |
| T7 terminator | CTAGTTATTGCTCAGCGG | — |
| TUB-F2 | CAGAGCCCC/GCTCAA | 82-95 |
| PAL-F1 | GCAGCTAAAGCCCTACTTCA | 276-295 |
| V.F1 | CAGCTTCTACGCCACCGG | 96-113 |
| TA-EX-F1 | TGTGAAGTACACAACCCTAGC | −1-20 |
| R29-REV1 | GCGCCGGCTGCGGGGG | 185-170 |
| V.R1 | CTGTGCTGTACCGGTGGCG | 123-105 |
| Pal-R1 | AGCATACCCTCCTTAGCCTC | 572-553 |

In addition, a nucleic acid sequence with SEQ ID NO. 1 and an amino acid sequence with SEQ ID NO. 2 are also called CanA, since both sequences encode a protein called Cannulae A. For the same reason, SEQ IDS NOS. 3 and 4 are called CanB; SEQ ID NOS. 5 and 6 are called CanC; SEQ ID NOS. 7 and 8 are called CanD; and SEQ ID NOS. 9 and 10 are called CanE.

TABLE 7
Plasmids Used In The Following Examples

| Plasmid | Size | Property | Reference |
|---|---|---|---|
| PBluescript ® II | 2.96 kb | AmpR; MCS flanked | Alting-Mees et al., |
| phagemid KS(−) | | by T3 and T7 | 1989; [Stratagene, |
| | | promoter; replication | Heidelberg] |
| | | vector | |
| pET17b | 3.31 kb | AmpR; MCS flanked | Studier et al., 1990; |
| | | by T7 promoter and | [AGS, Heidelberg] |
| | | T7 terminator; | |
| | | expression vector | |

### Example 1

### Media and Cultivation of Organisms

a) Anaerobic Cultivation of Hyperthermophilic Organisms in Serum Flasks

i. Preparation of Synthetic Sea Water (also called “SME”):

NaCl (27.70 g); MgSO₄×7H₂O (7.00 g); MgCl₂×6H₂O (5.50 g); KCl (0.65 g); NaBr (0.10 g); H₃BO₃ (0.03 g); CaCl₂×2H₂O (0.75 g); SrCl₂×6H₂O (15.00 mg); and KJ (0.50 mg) were added a Schott flask. To the Schott flask, H₂O_{bidist} was added until the total volume of the mixture in the Schott flask reaches 1,000 ml. After the complete dissolution of the chemicals, the mixture was gassed with nitrogen for 20 min. (max. 1 bar, color change of the nitrogen indicator resazurin from bluish purple to red). For the reduction, 20 ml of 2.5% (w/v) anaerobic Na₂S solution was injected per liter medium. After complete decoloration of the medium, the pH value was set, as desired, with 25% (v/v) anaerobic H₂SO₄.

Serum flasks (glass type III; Bormioli, Italy) were flushed twice with H₂O_{bidist} and dried at 100° C. for 2 hours. Then each flask was filled with 20 ml above medium in an anaerobic chamber (Coy-Lab Products; Ann Arbor, Mich., USA) under N₂/H₂ atmosphere (95/5; v/v), plugged with rubber stoppers and the rubber stopper were sealed with aluminum caps (“aluminum seal stoppers”; Belco Glass; New Jersey, USA). Prior to use, the rubber stoppers were boiled once in 0.2% HCl and twice in H₂O_{bidist} for one hour each. After autoclaving (thiosulfate in the medium; 20 min., 121° C., 2 bar) or vaporizing (sulfur in the medium; 1 hour, 100° C.), each of the serum flasks was evacuated three times alternatingly at a gas station and gassed aseptically with H₂/CO₂ (80/20, v/v, 2 bar).

ii. Medium for Pyrodictium abyssi (pH 5.5-6.0)

The medium contained SME (500.00 ml); KH₂PO₄ (0.50 g); Yeast extract (0.50 g); Na₂S₂O₃ (1.00 g); Resazurin (1%) (0.30 ml); and enough H₂O_{bidist} so that the total volume of the medium was 1,000 ml. The medium was autoclaved. The cultivation temperature was 102° C. The incubation of Pyrodictium abyssi was carried out while standing.

iii. Medium for Hyperthermus (pH 7.0)

The medium contained SME (500.00 ml); KH₂PO₄ (0.50 g); NH₄Cl (0.50 g); Sulfur (5.00 g); KJ (2.50 mg); NiSO₄×6H₂O (2.00 mg); Resazurin (1%) (0.30 ml); and enough H₂O_{bidist} so that the total volume of the medium was 1,000 ml. The medium was vaporized. Prior to inoculation, 6 g tryptone per liter were added in the form of an autoclaved stock solution (10%, w/v). The cultivation temperature was 100° C. The incubation of Hyperthermus was carried out while standing.

b) Media and Conditions for Escherichia coli

The diverse E. coli strains were routinely cultivated aerobically on LB₀ medium (see below) at 37° C. with intensive shaking (250 rpm). Plasmid-carrying strains with resistance to antibiotics were cultivated in the presence of the corresponding antibiotic (100 μg/ml) ampicillin, 34 μg/ml chloramphenicol).

i. LB₀ Medium for E. coli DN5α and BL 21 (DE3), (pH 7.0)

The medium contained Trypton (10.00 g); Yeast extract (5.00 g); NaCl (10.00 g); and enough H₂O_{bidist} so that the total volume of the medium was 1,000 ml.

ii. LB₀ Medium for E. coli Y1090 (pH 7.0)

The medium contained Trypton (10.00 g); Yeast extract (10.00 g); NaCl (5.00 g); and enough H₂O_{bidist} so that the total volume of the medium was 1,000 ml.

iii. NZYM Medium for E. coli Y1090 (pH 7.0)

The medium contained NZ amines (10.00 g); NaCl (5.00 g); Yeast extract (5.00 g); MgSO₄×7H₂O (2.00 g); and enough H₂O_{bidist} so that the total volume of the medium was 1,000 ml.

For the preparation of plates, 15 g agar per liter medium was used. 7.5 g agarose per liter medium was added to the Top Agar.

### Example 2

### Preparation Of Competent Cells

DH5α and BL 21 (DE3) cells were made competent with rubidium chloride for the uptake of plasmid DNA from the medium. The materials used as listed as following:

| | | |
|---|---|---|
| SOB: | | |
| Trypton | 5.00 | g |
| Yeast extract | 1.25 | g |
| 5 M NaCl | 0.50 | ml |
| 3 M KCl | 0.21 | ml |
| H₂O_{bidist} | up to 250.00 | ml |
| Glucose Solution (50×): | | |
| Glucose | 3.96 | g |
| MgSO₄ × 7 H₂O | 2.46 | g |
| MgCl₂ × 6 H₂O | 2.03 | g |
| H₂O_{bidist} | up to 20.00 | ml |

SOC Medium: 98 ml SOB + 2 ml 50 × glucose solution

| | | |
|---|---|---|
| Transformation | | |
| buffer: | TF I | TF II |

| RbCl | 1.20 | g | 36.00 | mg |
|---|---|---|---|---|
| MnCl₂ × 4 H₂O | 0.99 | g | — | |
| CaCl₂ × 2 H₂O | 0.15 | g | 0.33 | g |
| 87% glycerol | 15.00 | g | 4.50 | g |
| 1 M potassium | 3.00 | ml | — | |
| acetate (pH 7.5) | | | | |
| 0.5 M MOPS | — | | 0.60 | ml |
| H₂O_{bidist} | up to 100.00 | ml | up to 30.00 | ml |
| pH | 5.8 | | 6.8 | |

For TF I, the pH value was adjusted with acetic acid (15%). For TF II, the PH value was adjusted with a sodium hydroxide solution (5 M). The transformation buffer and the glucose solution were sterilized by filtration. The SOB medium was autoclaved.

First, 10 ml SOC medium was inoculated with a single colony of the desired E. coli strain and shaken at 37° C. overnight. 1 ml of this overnight culture was used as the inoculum for 100 ml SOC medium and incubated with shaking at 37° C. At an OD₆₀₀ of 0.4, the culture was distributed over three pre-cooled centrifuge beakers (JA 20 rotor). After standing for 15 minutes on ice, the cells were harvested (JA 20 rotor, 5 min. 7,000 rpm, 4° C.). The cell pellet of each beaker was absorbed in 11.4 ml ice cold TF I, put on ice for 15 min. and collected by centrifugation again (JA 20 rotor, 5 min, 7,000 rpm, 4° C.). Then each pellet was carefully resuspended in 2.9 ml ice cold TF II, proportioned (50 μl) and shock frozen in liquid nitrogen. The competent cells were stored at −80° C.

### Example 3

### Cell Lysis Buffer (pH 8.0)

The cell lysis buffer contained:

| | |
|---|---|
| Tris ® | 0.20 M |
| NaCl | 0.10 M |
| Na citrate | 1.00 mM |
| EDTA | 1.00 mM |

### Example 4

### Mechanical Cell Lysis

This cell lysis method was applied to Methanopyrus kandleri, Methanothermus fervidus and Pyrobaculum aerophilum.

In a precooled mortar approximately 0.5 g frozen cells were ground to a fine powder under liquid nitrogen. Following addition of 1-2 ml lysis buffer (see example 8) and thawing to room temperature, the suspension was introduced into an Eppendorf reaction vessel. Then the same procedure as described in example 10 was followed.

### Example 5

### Cell Lysis with Subtilisin

With the exception of the aforementioned organisms in Example 9, all organisms for DNA isolation were lysed as follows: 0.05-0.1 g cells were suspended with 500 μl lysis buffer (see example 8). Together with subtilisin (final concentration: 40 ng/μl) and 2 μl RNase, DNase-free, the suspension was incubated in the water bath at 37° C. for 30 minutes. Then the same procedure as described in Example 11 was followed.

### Example 6

### Phenol/Chloroform Extraction

This method of DNA cleaning was chosen for all organisms, whose DNAs were used for studying the propagation of cannulae genes. DNA solutions were pipetted with cut off pipette tips in order to largely avoid shear forces.

500 μl cell lysis (Examples 9 and 10) was treated with 500 μl buffer-saturated phenol and carefully mixed in an Eppendorf Reaction Vessel (ERV). For phase separation, the mixture was centrifuged in an Eppendorf centrifuge for 5 minutes at 13,000 rpm. After centrifugation, the DNA-containing solution (top layer) was transferred into a clean ERV, and treated with 205 μl phenol. Following careful swirling, 250 μl chloroform/isoamyl alcohol (24/1) were added, and the phases were mixed again. Following phase separation, the last step was repeated until there was no longer a white layer of proteins between the two phases. Finally the DNA suspension was treated with 500% chloroform/isoamyl alcohol (24/1, v/v), centrifuged for the last time, and the aqueous phase was transferred into a clean ERV.

To remove the phenol groups and to concentrate, the DNA was precipitated with ethanol. At the same time 1/10 volume 3M sodium acetate and 2.5 volume ethanol_{absolute} (−20° C.) were added; the DNA was precipitated at −80° C. for 30 min. and collected by centrifugation in a table centrifuge (30 min., 12,000 rpm, 4° C.). The pellet was washed with 200 μl 70% ethanol (−20° C.), centrifuged at 4° C. for 15 min., and dried in the desiccator for 15 min. Then the DNA was absorbed in 100 μl distilled water, treated with RNase, DNase-free (2 μl), and incubated for 30 min at 37° C. Then the DNA solution was stored at 4° C.

### Example 7

### CsCl Gradient Equilibrium Centrifugation

The DNA of the Pyrodictium abyssi isolate TAG11 was cleaned in the CsCl gradient by equilibrium centrifugation. One exception was the test for the genetic propagation of the cannulae genes. The same protocol was followed as described above. The DNA of 0.5 g Pyrodictium cells was resuspended in 1 ml H₂O_{bidist}.

### Example 8

Isolation Of Plasmid DNA From E. Coli
a). Buffer and Solutions used in this example

| | | | |
|---|---|---|---|
| S1 buffer: | Tris ®/HCl (pH 8.0) | 50 | mM |
| | EDTA | 10 | mM |
| S2 buffer: | NaOH | 200 | mM |
| | SDS | 1% | |
| S3 buffer: | KAc/HAc (pH 5.2) | 2.6 | M |
| N2 buffer: | Tris ®/H₃PO₄ (pH 6.3) | 100 | mM |
| | KCl | 900 | mM |
| | EtOH | 15% | |
| N3 buffer: | Tris ®/H₃PO₄ (pH 6.3) | 100 | mM |
| | KCl | 1150 | mM |
| | EtOH | 15% | |
| N5 buffer: | Tris ®/H₃PO₄ (pH 8.5) | 100 | mM |
| | KCl | 1000 | mM |
| | EtOH | 15% | |
| Binding solution: | guanidinium thiocyanate | 4 | M |
| | Tris/HCl (pH 7.5) | 50 | mM |
| | EDTA | 20 | mM |
| | binding matrix | 10 | mg/ml |
| Wash buffer: | NaCl | 200 | mM |
| | Tris/HCl (pH 7.5) | 20 | mM |
| | Na₂ EDTA | 5 | mM |

Prior to use, the wash buffer was diluted 1:1 with EtOH_{absolute}.

b). Preparation on the Mini Scale

Of the 10 ml E. coli overnight culture in LB₀ medium, 4 ml were collected by centrifugation in an ERV (table centrifuge, 3 min., 12,000 rpm). The pellet was resuspended in 100 μl S1 buffer and treated with 1 μl RNase, DNase-free, (0.5 mg/ml). Lysis took place by adding 200 μl S2 buffer at RT for 5 min. After neutralization with 200 μl S3 buffer, the batches were put on ice for 5 to 10 min. Then the chromosomal DNA, cell groups and precipitated DKS were pelletized (table centrifuge, 5 min., 12,000 rpm). The supernatant was mixed with 1 ml binding solution and incubated at RT for least 20 min. In the interim the sedimented binding matrix was agitated several times. Then collection by centrifugation followed (table centrifuge, 2 min., 12,000 rpm); and the supernatant was discarded. After washing twice in 1.5 ml wash buffer each, the pellet was dried in the desiccator for 15 min. and resuspended in 120 μl H₂O_{bidist}. For quantitative elution of the DNA, the suspension was incubated at 60° C. for 10 min. After slowly cooling, the binding matrix was sedimented (table centrifuge, 5 min., 12,000 rpm) and the plasmid-containing supernatant was transferred into a new ERV.

### Example 9

### Analysis and Cleaning of DNA

a) Concentration Measurement

i. Photometric Determination

The concentration of dissolved DNA was determined by measuring the optical density (OD) at 260 nm. A 1:20 dilution of the DNA solution was used. From the measured value, the concentration of the undiluted DNA solution was then determined:
OD₂₆₀ nm of the 1:20 dilution≈μg/μl [DNA_{undiluted}]

ii. Ethidium Bromide Plates

If there were only very low concentrations or absolute quantities of DNA, then they were estimated by comparing with the standard concentrations.

| | | | |
|---|---|---|---|
| Plates: | agarose | 5.0 | g |
| | 1 M Tris/HCl (pH 7.5) | 5 | ml |
| | 0.5 M EDTA (pH 8.0) | 1 | ml |
| | ethidium bromide (10 mg/ml) | 0.25 | ml |
| | H₂O_{bidist} | up to 500 | ml |

The agarose was dissolved in water by boiling. After cooling to approx. 60° C., the remaining components were added. The solution was poured into Petri dishes (Sarstedt, Ulm). Following solidification, 1 μl each of the DNA solution of unknown concentration was pipetted to the plates in parallel with DNA standards (10-100 ng/μl). After approx. 5 minutes, the fluorescent intensity of the standard and of the sample in UV light was compared and thus the unknown concentration was estimated.

The finished plates can be stored under light protection for several weeks at 4° C.

b) Agarose Gel Electrophoresis

i. Buffer and Solutions

| | | | |
|---|---|---|---|
| TAE running | Tris/acetate pH (8.35) | 400 | mM |
| buffer (10×): | Na₄ EDTA | 10 | mM |
| Application | EDTA | 50 | mM |
| buffer: | saccharose | 40% | |
| | bromophenol blue | 0.1% | |
| | xylene cyanol | 0.1% | |

ii. Protocol

For the analysis of PCR products, plasmids, and genomic DNA, 0.8-2.5% agarose gels were used. In the subsequent elution from the gel (see example 14.b), a low melting agarose was used. Sea-Kem agarose was used, when the DNA was blotted on a membrane following electrophoretic separation (see example 19.d).

The agarose was dissolved in H₂O by boiling. After cooling under flowing water and addition of 1/10 volume 10×TAE and 1/10,000 volume ethidium bromide (10 mg/ml), the gel solution was poured into a horizontal gel chamber (30 ml: 7×10 cm or 200 ml: 20×22 cm). The samples were treated with 1/5 volume application buffer prior to application. The gel run took place in 1×TAE at 80-120 V for 30-90 minutes. The separation was controlled on a UV fluorescent screen and evaluated and documented with an EASY image analysis system (Herolab, Heidelberg).

c) Isolation of DNA from Agarose Gels

To isolate single restriction fragments, the batches were separated using an agarose gel (1%) with a special, low melting agarose. The desired bands were cut out under UV light and the agar blocks were weighed (1 mg≈1 μl).

After being filled with H₂O_{bidist} up to 9/10 reaction volume, and being added with 1/10 volume 10×agarase buffer, the agar block was melted with frequent, intensive shaking at 65° C. for 10 min. After 5 min. pre-incubation at 40° C., 1 μl β-agarase (1 unit) was added to the melted agar block to form a mixture. The mixture was incubated for another hour at 40° C., during which period there was frequent mixing. The mixture was put on ice for 10 min. and then collected by centrifugation in a table centrifuge at 12,000 rpm at RT for 10 min. The DNA was precipitated from the supernatant with ethanol (see example 11).

### Example 10

### Polymerase Chain Reaction (PCR)

The reaction was conducted in 0.2 ml reaction vessels (Stratagene, Heidelberg). Upon the start of the reaction, the reaction was kept on ice and the DNA polymerase was always added last. The batches were coated with the same volume of Chill-out 14™ liquid wax (MJ Research, Inc., Nalgene) in order to check the evaporation during the reaction. (After setting up the Thermo-Cycler with a heatable cover, this coating was no longer necessary.) The amplification took place in a Robocycler (gradient 96, Stratagene). The PCR products were cleaned with the High Pure PCR Purification kit from Boehringer (Mannheim) and analyzed by agarose gel electrophoresis (see example 14.b).

a) Standard PCR

To amplify specific segments of the chromosomal DNA and to estimate the size and orientation of the insert for plasmids, cleaned DNA was used as the matrix.

| | | | |
|---|---|---|---|
| Reaction batch: | Taq PCR buffer (10×) | 2.5 | μl |
| | dNTP (per 2.5 mM) | 2.0 | μl |
| | primer A (20 pMol/μl) | 0.5 | μl |
| | primer B (20 pMol/μl) | 0.5 | μl |
| | plasmid DNA (5 ng/μl) | 2.0 | μl |
| | Taq DNA polymerase (5 U/μl) | 0.13 | μl |
| | H₂O_{bidist} | 17.37 | μl |
| Taq PCR | Tris/HCl (pH 8.3) | 100 | mM |
| buffer (10×): | KCl | 500 | mM |
| | MgCl₂ | 15 | mM |

Program: 3 min 95° C., 32×(1 min. 95° C., 1 min. 55° C., 1.5 min. 72° C.), 10 min 72° C.

For PCR products that were more than 1,500 bp long, the polymerization time (72° C.) per 1,000 bp was increased by 1 minute.

With the addition of chromosomal DNA, 50 ng were used as the matrix.

b) PCR Screening

This method was used to check the insert size of diverse clones by means of PCR. Used was the primer pair M13 lac Z (reverse and forward, Perkin Elmer), which bind to the flanking regions of the multiple cloning site of the KS(−) vector. Either 5-10 ng cleaned plasmid DNA or whole plasmid-containing cells were added as the matrix (to this end, the colonies were picked from the LB₀ plate with sterilized toothpicks).

Program: 5 min 95° C., 32×(1 min. 95° C., 1 min. 55° C., 2-5 min. 72° C.), 10 min 72° C.

c) Introduction of Restriction Sites with PCR

To construct expression plasmids, DNA fragments had to be inserted into the expression vector (pET17b) in a precisely defined reading frame. Therefore, it was necessary to insert new restriction sites at the 5′ and 3′ end of the protein-coding DNA segment. For this reason, the gene was amplified with two primers, which contained the respective restriction sites at the corresponding places. At translation start (ATG), a NdeI site (CATATG) was inserted; after the translation stop (TAA) a NotI site (CGCCGGCG) was inserted. The resulting PCR product could then be inserted into the expression vector by means of the newly created restriction sequences. To guarantee the minimum probability of error in the DNA synthesis, Pfu-DNA polymerase was used here. It contains a 3′->5′ exonuclease activity (proofreading), which enables the splitting off of the nucleotides that have been incorrectly incorporated at the 3′ end of the synthesized DNA strand.

| Batch: | | |
|---|---|---|
| pfu-PCR buffer (10 ×) | 2.5 | μl |
| dNTP(per 2.5 mM) | 2.0 | μl |
| primer * EX-F * (20 pMol/μl) | 0.5 | μl |
| primer * EX-R * (20 pMol/μl) | 0.5 | μl |
| plasmid DNA (5 ng/μl) | 1.0 | μl |
| Pfu-DNA polymerase (2.5 U/μl) 0.26 | μl | |
| H₂O_{bidist} | 18.24 | μl |

| | |
|---|---|
| | Program: |
| CanA: | 3 min 95° C., |
| | 32 × (1 min. 95° C., |
| | 1 min. 20 s 65° C., |
| | 1 min. 15 s 72° C.), |
| | 10 min 72° C. |
| CanB: | 3 min 95° C., |
| | 32 × (1 min. 95° C., |
| | 1 min. 20 s 63° C., |
| | 1 min. 15 s 72° C.), |
| | 10 min 72° C. |
| CanC: | 3 min 95° C., |
| | 32 × (1 min. 95° C., |
| | 1 min. 20 s 55° C., |
| | 1 min. 15 s 72° C.), |
| | 10 min 72° C. |

| | |
|---|---|
| | Expression primer: |
| CAN-EX-FA/B: | 5′-TAGCAGGCCATATGACCACCCAGAGCCCCC-3′ |
| CAN-EX-FC: | 5′-CTAGCAGGCCATATGACGACCCAGAGCC-3′ |
| CAN-EX-RA: | 5′-GGAGGACTGGCGGCCGCTGTTAGCCTAC-3′ |
| CAN-EX-RB: | 5′-AGTAGCTAGCGGCCGCTTTAGCTGACGC-3′ |
| CAN-EX-RC: | 5′-GGCCGTGGCGGCCGCTGCTTCACC-3′ |

The inserted restriction sites are underlined.

d) RT PCR

RT PCR is one of the most sensitive methods to determine the presence or absence of specific RNA molecules or to quantify the strength of the gene expression. In contrast to a normal PCR, in a RT PCR process, RNA is used as the matrix, which can be translated back into DNA by reverse transcriptase (RT). The next step of the RT PCR process is a “normal” PCR, where the newly synthesized DNA is used as a template and is amplified.

In the present study, a Titan™ One Tube RT PCR system (Boehringer, Mannheim) was used. In the first step of the RT PCR process, AMV reverse transcriptase was used for the first strand synthesis. An Expand™ High Fidelity Enzyme Mix (Taq DNA polymerase and Pwo DNA polymerase) is used for the “normal” PCR step of the RT PCR process. The following batch was made according to the standard:
Master mix 1: 4 μl dNTP (per 2.5 mM), 41 μl primer 1 (5 μM/μl), 4 μl primer 2 (5 μM/μl), 2.5 μl DTT (100 mM), 6 μl RNase inhibitor (1 U/μl), 1 μl mRNA (1 pg-1 μg), up to 25 μl DEPC-H₂O
Master mix 2: 10 μl 5×RT buffer with Mg²⁺, 1 μl enzyme mix, up to 25 μl DEPC-H₂O

The two master mixes were combined, mixed, centrifuged and put into the preheated (60° C.) block of the thermocycler. Program: 30 min. 60° C., 2 min. 94° C., 10×(1 min. 94° C., 1 min. 55° C., 1 min. 15 s 68° C.), 20×(1 min. 94° C., 1 min. 55° C., 1 min. 35 s 68° C.), 5 min 72° C.

### Example 11

### Cloning Of DNA Fragments

a) Restriction Hydrolysis

The double stranded DNA was cleaved with restriction enzymes for at least two hours at 37° C. in the water bath.

b) Dephosphorylation of DNA Fragments

To suppress the religation of linearized vectors, the sites at the 5′ end were dephosphorylated with alkaline calf intestine phosphatase (CIP). To this end, the restriction batches were filled, according to the standard, up to 45 μl with H₂O following phenol/chloroform treatment and DNA precipitation (see example 11). 5 μl 10× phosphatase buffer (0.5 M TRIS® (hydroxymethyl amino methane hydrochloride) (pH 9.10), 10 mM MgCl₂, 1 mM ZnCl₂, 10 mM spermidine) and 1 μl CIP (1 U/μl) were added and incubated at 37° C. for one hour. After a second addition of CIP (1 μl), the incubation was continued for another hour. Then the batches were phenol-extracted and precipitated with ethanol (see example 11).

c) Filling up of Overhanging Ends

The ends of the PCR products or restriction fragments were filled in with T7 polymerase. For example, 50 μl cleaned restriction batch were treated with 5 μl H₂O, 7 μl restriction buffer (Boehringer, Mannheim), 6 μl dNTP (per 2.5 mM), and 2 μl T7 polymerase and incubated in the water bath at 37° C. for one hour. After inactivation of the polymerase (20 min, 65° C.), the batch was cleaned with the High Pure PCR Purification kit from Boehringer (Mannheim).

d) Production of a T Vector

To clone the PCR products, a so-called T vector was produced. For example, the vector pBluescript KS(−) was linearized with EcoRV (see example 16 a)) and then incubated in the presence of 2 mM dTTP with Taq polymerase (1 U/μg vector) at 70° C. for 2 hours. The reaction took place under standard buffer conditions (50 mM KCl, 10 mM TRIS® (hydroxymethyl amino methane hydrochloride) (pH 8.3), 1.5 mM MgCl₂ and 200 μg/ml BSA). The reaction volume was 20 μl. Following phenolation and ethanol precipitation (see example 11), the T vector was resuspended in TE buffer (10 mM TRIS® (hydroxymethyl amino methane hydrochloride) (pH 8.0), 1 mM EDTA); and a concentration of 60 ng/μl was set.

e) Ligation of DNA Fragments

100-120×10⁻¹⁵ mole fragment and 30-40×10⁻¹⁵ mole digested vector DNA were transferred into a 10 μl vessel. The bonding took place in a buffer, provided by the manufacturer, with 1 U T4 DNA ligase overnight at 16° C.

f) Transformation

50 μl competent cells were thawed on ice, 2 μl 0.5 M 2-mercapto ethanol and 3 μl ligation batch (see example 16.e) were added to the competent cells and carefully stirred with the pipette tip. Then the mixture was incubated on ice for 30 min. After 30 s at 42° C., the mixture was put on ice again for 1-2 minutes. After addition of 450 μl fresh sterile SOC medium (see example 7), the mixture was temperature controlled at 37° C. in the water bath for 1-2 minutes for fast temperature conformation. The transformation mixture was shaken at 37° C. for 60 min. and then plated out repeatedly 200 μl per LB₀ plate (treated with 100 μl ampicillin (10 mg/ml), 100 μl X-gal (20 mg/ml in formamide) and 10 μl IPTG (0.1 M)). The plates were incubated at 37° C. overnight. The pretreatment with X-gal and IPTG allowed a blue/white screening of the transformants. Colonies of transformants with an insert in the incorporated vector appeared white; without the insert, blue.

g) Glycerol Cultures

Long-term cultures, also called glycerol cultures, were prepared from the transformed E. coli strains. For example, 2 ml overnight culture pellet were collected by centrifugation; the pellet was resuspended in 140 μl fresh LB₀ (see example 6.b), thoroughly mixed with 200 μl sterile glycerol (87%) and deep frozen at −80° C.

### Example 12

### Sequencing

a) Plasmids

The sequencing reaction was conducted with the Sequenase Quick Denature™ Plasmid Sequencing kit from USB. In contrast to the manufacturer's recommended termination reaction temperature, the termination reactions were conducted at 45° C. (thermoblock). The radioactive marking was done with ³⁵S-dATP.

b) PCR Products

The sequencing reactions were conducted with the AmpliCycle™ Sequencing kit from PERKIN ELMER in a thermocycler. The radioactive marking was done with ³³P-dCTP.

| | | | |
|---|---|---|---|
| Annealing mix: | PCT product (cleaned) | 100 | ng |
| | primer | 10 | pMol |
| | H₂O | up to 15 | μl |
| Cycling master mix: | H₂O | 10.75 | μl |
| | α-³³P-dCTP (10 μCi) | 0.25 | μl |
| | cycling mix | 4.00 | μl |

2 μl of each of the termination mixes was transferred into a 0.2 ml PCR tube on ice.

The annealing mix and cycling master mix were combined and mixed to form a mixture. 6 μl of this mixture was pipetted (on ice) to each of the termination mixes in the PCR tubes. The PCR tubes were then transferred to the preheated thermocyclers and the program was started. At the end of the program, 4 μl stop solution was added and the samples in the PCR tubes were frozen until gel application.

Program: 2 min 94° C., 32×(1 min 94° C., 75 s 55-65° C., 65 s 72° C.), 5 min 72° C. The annealing temperature varied as a function of the oligonucleotide that was used.

c) Phage DNA

To sequence phage DNA, the same protocol as described in example 17.b was followed. However, instead of 100 ng PCR product, 1 μg phage DNA was added to the annealing mix.

Program: 2 min 94° C., 32×(1 min 94° C., 75 s 50° C., 65 s 72° C.), 5 min 72° C.

d) Polyacrylamide Urea Gel Electrophoresis

The electrophoretic separation of the single strand DNA after sequencing reactions was done under denaturing conditions over 6% polyacrylamide urea gels. The exact composition and procedure has already been described by Mai B. in “Genetic Characterization and Expression of the Large Thermosome Subunit from Pyrodictium occultum in E. coli and Molecular Biological Studies on the Extracellular Network form Pyrodictium abyssi Isolate TAG 11,” Thesis from the Department of Microbiology at the University of Regensburg (1995).

### Example 13

### Bacteriophages: Lysates and DNA Preparation

a) Titer Determination of Phage Lysates

To determine the number of phages per ml lysate (plaque forming units, pfu), dilution series (10⁻² to 10⁻⁸) in SM buffer (50 mM TRIS® (hydroxymethyl amino methane hydrochloride) (pH 7.5), 100 mM NaCl, 10 mM MgSO₄) were prepared from the lysate. 100 μl at a time were plated out as follows. The dilution was mixed with 100 μl host cell culture (E. coli Y1090, OD₆₀₀=1.0 in 10 mM MgSO₄), incubated at 37° C. for 30 minutes and the entire batch was added to 3 ml NZY Top agar (see example 6, melted at 100° C. and cooled to 48° C.). Following fast mixing, the Top agar was poured immediately and uniformly on preheated NZY plates. Bacteria races and plaques developed overnight at 37° C. The phage titer in the lysate could be determined by counting out and by taking dilution factors into consideration.

b) Isolation of Phage Plaques

To separate the bacteriophages with the desired DNA sequence from others, they were first isolated by plating out (10.1) 200-400 pfu per NZY plate (diameter 9 cm). The desired plaques were picked out with a sterile glass Pasteur pipette and transferred into 100-200 μl phage buffer (20 mM TRIS® (hydroxymethyl amino methane hydrochloride) (pH 7.4), 100 mM NaCl, 20 mM MgSO₄). The phages were diffused from the agar either in one hour at 37° C. or overnight at 4° C. For longer storage at 4° C., a drop of chloroform was added to keep it sterile.

c) Preparation of λ Phages (Liquid Culture Method)

500 μl fresh overnight culture from the host strain E. coli (single colony in 10 ml LBₒ with 0.2% maltose and 10 mM MgSO₄) were quickly and thoroughly mixed with 20 μl phage solution (10.2≈10⁵ pfu) and incubated in the water bath at 37° C. for 20 minutes.

Then the mixture with the infected cells (the host strain E. Coli with phages) was added to 100 ml preheated LB₀ (37° C. with 1 mM MgSO₄ and 10 mg ampicillin) and intensively shaken at 37° C. Five to seven hours later, the cell lysis had taken place. It had taken place with regular measurements of OD₆₀₀ during incubation. To clarify the culture (=cell lysis), 500 μl chloroform were added and shaken for another 15 minutes. The cell fragments were removed by centrifugation (JA 10 rotor, 7,000 rpm, 10 min); and the phage-containing supernatant was transferred into sterile vessels and stored at 4° C.

d) Isolation of the Phage DNA

The phase DNA was isolated from 10 ml lysate (10.3) with the Wizard™ Lambda Preps DNA Purification system (Promega, Mannheim).

### Example 14

### Identification of Desired DNA Sequences

a) Preparation of DIG-Marked Probes

DIG-11-dUTP (digoxygenin or DIG) is a substrate for the E. coli DNA polymerase, T4 DNA polymerase, Taq DNA polymerase and reverse transcriptase. It may be used in the “nick translation” reaction and the “random primed DNA labeling” method in place of dTTP for DNA marking (DIG-11-dUTP: dTTP=35%:65%). The DIG-marked DNA can then be identified using the following procedure.

i. DIG-11-dUTP Incorporation into PCR Products

During a standard PCR (see Example 15) 2 μl DIG-11-dUTP (1 mM) were added to the batch.

ii. “Random Primed DNA Labeling” Reaction

The finished PCR product was marked according to the instructions provided by Boehringer, Mannheim. For example, starting from random primers, different sizes of segments of a DNA are synthesized using Klenow polymerase, whereby DIG-11-dUTP is incorporated. The size of the DIG-marked DNA fragments, which are obtained in the “random primed” DNA marking process, depends on the quantity and the length of the matrices-DNA. Every 20th to 25th nucleotide of the freshly synthesized DNA is a DIG-11-dUTP.

15 μl cleaned PCR product (1.5 μg; made in example 15) were boiled in the water bath for 10 min. and then quickly cooled on an ice NaCl mixture, since a complete denaturing turned out to be especially important for effective marking. 2 μl hexanucleotide mixture (10×), 2 μl DIG DNA Labeling Mix (10×) and 1 μl Klenow enzyme (2U) were added; and the mixture was incubated at 37° C. for two hours. Then the reaction was stopped by adding 2 μl 0.2 M EDTA (pH 8.0) and 2.5 μl 4 M LiCl₂. The marked DNA was precipitated with ethanol and dissolved in 50 μl TE buffer (10 mM TRIS® (hydroxymethyl amino methane hydrochloride) (pH 8.0), 1 mM EDTA) at 37° C. (30 min.).

b) Detection in E. coli Transformants

i. Colony Transfer (“Colony Lift”)

To detect positive colonies following transformation (see example 16.f), up to 100 transformants were inoculated on two identical LB₀ scanning plates with suitable antibiotic addition and incubated at 37° C. overnight. A dry nylon membrane (Hybond™-N⁺, Amersham, Braunschweig) was laid on the grown colony at RT for 3 minutes, after the plates had been stored at 4° C. for four hours. Then the membrane was laid on a NaOH-saturated (0.5 M) Whatman 3 MM paper with the colony side up for 5 min., then 2 minutes on dry and once again 5 min on a NaOH-saturated Whatman 3 MM paper. Finally the alkaline denatured DNA was fixed on the membrane (120° C., 45 min.). Through hybridizing the membrane with a DNA probe (see example 19.a) and detecting DIG with chemiluminescence (see example 19.f), the transformants with the desired DNA sequence could be identified on the scanning plate and inoculated from the second plate.

ii. Plasmids and Phage DNA

Isolated plasmid and phage DNA were checked as follows. DNAs with predetermined concentrations (1 pg up to 100 ng plasmid, 1 ng up to 10 μg phage DNA) were dapped on a dry nylon membrane (Boehringer, Mannheim). For comparison purposes, the appropriate controls (e.g. vector without insert) were always carried out at the same time. As described in example 19.b.i), the applied DNA was denatured with alkaline and fixed. Then the DNA on the membrane was hybridized with the appropriate probe overnight (see example 19.e) and the DIG-marked DNA was detected (see example 19.f).

c) Identification in Bacteriophages

i. Phage Mixtures (“Plaque Lift”)

If the desired DNA sequence was identified in lysates with different phages (e.g. in the gene bank), then 200 to 400 pfu in NZY Topagar was plated out on NZY plates (see example 6). As described for the bacteria colony (example 19.b.i), the phages were then transferred onto a nylon membrane; the DNA was released with NaOH, denatured and then heat fixed. The DIG identification was directly conducted colorimetrically (see example 19.f) on the membrane in order to facilitate the allocation of signal and plaque. Then the identified plaques could be isolated from the plate (see example 18.b).

ii. Mini Lysates

9 μl lysate was treated with 1 μl 2 M NaOH and 2 mM EDTA and incubated at RT for 10 minutes. Then 2 μl per batch was pipetted on a dry nylon membrane (Boehringer, Mannheim). After 30 minutes at 120° C., the membrane was hybridized with the corresponding probe. The DIG was identified with chemiluminescence (see example 19.f).

d) Identification in Restriction-Digested DNA (Southern Blot)

| | |
|---|---|
| TAE running buffer: | 40 mM Tris ®/acetic acid (pH 8.4), 10 mM EDTA |
| Denaturing buffer: | 0.5 NaOH, 1.5 M NaCl |
| Neutralizing buffer: | 1 M Tris/HCl (pH 7.5), 1.5 M NaCl |
| 10 × SSC: | 1.5 M NaCl, 0.15 M Na citrate, (pH 7.0) |

First, the restriction-digested DNA (see example 16.a) was separated on a 1% SeaKem agarose gel in TAE buffer (see example 16.b) and photographed (together with a ruler as the scale). The gel was incubated for 8 min in 0.25 M HCl, then 20 min in denaturing buffer and finally incubated in neutralizing buffer for 20 minutes. In the interim a nylon membrane (Boehringer, Mannheim) and two Whatman filters (3 MM), which had been soaked in 10×SSC for 1 minute just before use, were cut to fit the size of the gel.

The DNA fragments were then transferred to a positively loaded nylon membrane with a POSI BLOT 10-30™ (Stratagene, Heidelberg). A moist Whatman paper and the wetted membrane were laid on the rough side of the blot apparatus. Over this was laid a plastic template, whose edges were approximately 0.5 cm smaller than the gel. The pretreated gel was placed on the template in such a manner that the application wells rested on the plastic and the opening of the template was completely covered. Another Whatman paper was put on the gel. Finally a wet sponge (10×SSC) was put on the top. Excess pressure (70-80 mm Hg) was applied on the sponge for one hour.

Then gel traces and start line were marked on the membrane and the transferred DNA was fixed at 120° C. for 30 minutes. Following hybridization (see example 19.e) and DIG detection (see example 19.f), the fragments with the desired DNA sequence could be clearly identified (at split plasmids or phage DNAs) or at least assigned to a specific size range (for digestion of chromosomal DNA).

e) Hybridization with DIG Probes

In a hybridization buffer DIG EASY HYB™ (Boehringer, Mannheim), a probe concentration of 20 ng/ml was set. A DIG-marked probe was denatured at 100° C. for 5 minutes and cooled on ice. The hybridization solution was used multiple times. Between the individual hybridizations it was stored at −10° C. and denatured at 68° C. for 15 minutes prior to be reused. DIG EASY HYB™ contains no formamide. However, the hybridization temperature was analogously calculated to the formamide-containing hybridization solution (50%). Typically, a hybridization temperature ranging from 43-50° C. was determined for the Pyrodictium probes. To detect homologous genes with the probes, the hybridization temperature was decreased (Pyrodictium DNA: 42° C.; DNA of other organisms: 34° C.). After 30 minutes pre-hybridization (without probe) the batch was hybridized overnight, then washed 2×5 min in 2×SSC with 0.1% SDS (w/v) at room temperature. Finally the membrane was shaken for 2×15 min. longer in 0.1×SSC with 0.1% SDS (w/v) at 68° C. (Pyrodictium DNA) or 60° C. (DNA of other organisms).

f) Detection of DIG-Marked DNA

| | |
|---|---|
| Buffer 1: | 0.1 M maleic acid/NaOH (pH 7.5), 0.15 M NaCl |
| Wash buffer: | 0.3% (v/v) Tween 20 in buffer 1 |
| Buffer 2: | 1% (w/v) blocking reagent in buffer 1 |
| Buffer 3: | 0.1 M Tris/HCl (pH 9.5), 0.1 M NaCl, 50 mM MgCl₂ |
| NBT solution: | 75 mg NBT in 1 ml 70% dimethylformamide |
| BCIB solution: | 50 mg BCIP in 1 ml dimethylformamide |

The membrane was first shaken in the wash buffer for 2-5 minutes. Then the free binding sites on the membrane were saturated with buffer 2 for 30 minutes. Thereafter, the anti-DIG alkaline phosphatase conjugate was diluted in buffer 2 (1:10,000). The membrane was then incubated in the diluted anti-DIG alkaline phosphatase conjugate for 30 minutes. Unbound antibody conjugates were removed by 2×15 min. shaking in the wash buffer. Then the membrane was equilibrated in buffer 3 for 3 minutes.

Colorimetric Detection:

90 μl NBT and 70 μl BCIP solution were added to 20 ml buffer 3 to form a mixture. The membrane was coated with the mixture and left standing in the dark to incubate (30-120 min). The reaction (violet-brownish coloration) was terminated by placing the membrane in water.

Chemiluminescence Detection:

CDP-Star™ chemiluminescence substrate was diluted 1:10 in buffer 3 and inserted together with the membrane into a plastic sheet. The DIG-marked DNA was made visible with an x-ray film (Biomax MR1, Kodak, applied for 3 min-12 hours).

### Example 15

### Expression Of Recombinant Proteins In E. coli

a) Expression System that was Used

To express foreign proteins in the E. coli strain BL21 (DE3), the vector pET17b was used. The expression strain BL21 (DE3) pLysS accommodates the lysogenic phage DE3, which exhibits in turn the T7 RNA polymerase gene under the control of the lacUV5 promoter. The induction of this promoter with IPTG results in the synthesis of the T7 RNA polymerase, which, starting from the T7 promoter on pET17b, causes at this stage the transcription of the incorporated genes. The plasmid pLysS, which is also contained in the expression strain, carries not only a chloramphenicol resistance gene but also the gene for T7 lysozyme, an inhibitor of T7 RNA polymerase. Of course, the lysozyme gene is expressed only weakly, thus inhibiting the polymerase, formed in small quantities, in non-induced cells. This inhibiting effect can be easily overcome through induction of the polymerase. Thus, pLysS does, in fact, suppress the basal expression of foreign genes, but does not have a negative effect on the expression after induction.

b) Protocol

First of all, the vector pET17b was linearized with NdeI and NotI (see example 16.a) and dephosphorylated with CIP (see example 16.b). Then the NdeI and NotI sites were attached to the genes to be expressed by PCR (see example 15.c). The formed PCR products were cleaved with NdeI and NotI (see example 16.a), separated on an agarose gel and isolated (see example 14.c). The fragments (vector and insert) prepared thus were ligated (see example 16.e) and transformed in DH5α cells (see example 16.f). The transformants were checked for their insert size (see example 15.b). The resulting plasmid such as pEX-CAN-A was prepared from suitable transformants (see example 13); and for the control the transition sites from the vector to the insert were sequenced (see example 17.a). Then the transformation in BL21 (DE3) took place (see example 16.f).

To express the cannulae genes such CanA, CanB, CanC, CanD, CanE or sequences substantially identical thereof, the following procedure was followed:

A transformant pre-culture (2.5 ml LB₀ with ampicillin) was shaken up to an OD₆₀₀=1.0 at 37° C. and stored at 4° C. overnight. The next day this pre-culture was removed by centrifugation at 12,000 rpm in an ERV for 30 s. The pellet was resuspended in 2 ml fresh LB₀. Thus 50 ml LB₀ medium (+ampicillin) was inoculated. This medium was incubated with shaking at 37° C. The growth was monitored by routine OD measurement. At OD₆₀₀=0.6, 80 μl were removed. Then with the addition of IPTG (final concentration 0.3 mM) the T7 RNA polymerase was induced. Every 30-45 min. the OD₆₀₀ was measured; and 40 μl samples were removed. The cell samples were removed by centrifugation, resuspended in 10 μl application buffer (see example 22.a.i), and stored at −20° C. until the application on an SDS polyacrylamide gel (see example 23.a). As the control, a parallel batch with BL 21 (DE3) was inoculated with pET17b (without the insert) and prepared similarly. The cell harvest (JA 20 rotor, 9,000 rpm, 10 min, 4° C.) took place 3.5 hours after induction.

### Example 16

Isolating Recombinant Proteins From E. coli

| | |
|---|---|
| Low salt buffer: | 80 mM NaCl, 50 mM Tris/HCl (pH 7.5), 9% glycerol |
| High salt buffer: | 1.2 M NaCl, 50 mM Tris/HCl (pH 7.5), 9% glycerol |

a) CanA and CanB

One gram of recombinant E. coli with a particular sequence such as CanA or CanB expressed was absorbed in 4 ml low salt buffer. Cell lysis was conducted with a French press (2×at 20,000 psi, American Instrument Co., Silver Spring, USA). After pelletizing the cell fragments (Eppendorf centrifuge, 13,000 rpm, 5 min., RT), the protein solution was incubated at 80° C. for 20 min. Then the denatured proteins were removed by centrifugation (as above). The supernatant was passed at 1 ml/min through a Q sepharose column (1×12 cm=9.4 ml, Pharmacia, Freiburg). The eluent containing CanA or CanB was collected. The collected eluant was treated with leupeptin (1 μg/μl) and concentrated by a factor of 3-4 (based on the volume) in 4-8 hours in the MacrosepT centrifuge concentrators (Pall Filtron, Dreieich) with an exclusion limit of 5 kDA. After determining the protein concentration with the BCA test (see example 22.b.i), the purified protein was shock frozen in liquid nitrogen in 100-200 μl aliquots and stored at −80° C. In each working step, a sample was taken and analyzed on an SDS polyacrylamide gel (see example 22.a).

b) CanC

The first step of isolating CanC is same as that of CanA and CanB (see example 16.a). However, during the second step, CanC was retained on the Q sepharose. After flushing the column with low salt buffer, CanC was eluted from the column with a salt gradient (80-750 mM, in 60 ml) and collected by fractionation (1 ml each). Following analysis of the individual fractions on an SDS polyacrylamide gel (see example 22.a), the CanC-containing fractions were combined and dialyzed against the low salt buffer at 4° C. overnight. Finally the protein solution was eluded at 1 ml/min through a 1 ml ResourceQ column (Pharmacia, Freiburg). Then a salt gradient (80-750 mM, in 60 ml) was applied and 0.5 ml fractions were collected. After analysis of the same on an SDS polyacrylamide gel (see example 22.a), the CanC-containing fractions were combined again and dialyzed against low salt buffer overnight. Following addition of leupeptin (1 μg/μl), the solution was concentrated by a factor of 7 (based on the volume) in 6 hours in the MICROSEP™ centrifuge concentrators (Pall Filtron, Dreieich) with an exclusion limit of 5 kDa. The rest of the protocol is same as those described in example 21.a.

### Example 17

### Analysis Of Protein Solutions

a) SDS Polyacrylamide Gel Electrophoresis (Laemmli, 1970)

i. Solutions that were Used

| | | | |
|---|---|---|---|
| Running buffer (5×): | Tris | 25 | mM |
| | glycine | 250 | mM |
| | SDS | 0.1% | |
| Application buffer (1×): | Tris/HCl (pH 6.8) | 50 | mM |
| | SDS | 2% | |
| | 2-mercapto ethanol | 5% | |
| | glycerol | 10% | |
| | bromophenol blue | 0.1% | |

Gel Solutions (Volume in μl):

| | | 3% | 5% | 25% |
|---|---|---|---|---|
| | | Collection | Separation | Separation |
| | Gel Seal | Gel | Gel | Gel |
| 1 M Tris (pH 8.8) | 250 | — | 1250 | 1250 |
| 1 M Tris (pH 6.8) | — | 1250 | — | — |
| H₂O bidist | 285 | 7500 | 2900 | — |
| 60% acrylamide | 330 | 500 | 420 | 2100 |
| 2.5% bisacrylamide | 85 | 610 | 400 | 1200 |
| 10% SDS | 10 | 100 | 50 | 50 |
| 85% glycerol + BPB | — | — | — | 400 |
| TEMED | 1 | 10 | 1 | 0.5 |
| 30% APS | 10 | 70 | 5 | 5 |

ii. Protocol

To separate denatured proteins according to their size, SDS polyacrylamide gels were used. Separating gels (8.5 cm×6.5 cm, thickness 0.75 mm) having a linear acrylamide gradient ranging from 5 to 25% were poured. Following polymerization for one hour, a 3% collection gel was layered over the separating gel; and a comb with 10 application wells was inserted. The samples were absorbed in 10 μl application buffer, heated in the boiling water bath for 4 min. and applied with an extended pipette tip.

Electrophoresis was conducted at a constant current strength of 20 mA/gel (Mighty Small SE 250; Hoefer, San Francisco, USA). As soon as the bromophenol blue front had reached the bottom gel edge, the gel run was terminated.

b) Coomassie Staining of SDS Gels

| | | |
|---|---|---|
| Staining solution: | Coomassie R 250 | 0.1% |
| | methanol | 30% |
| | glacial acetic acid | 10% |
| Destainer: | methanol | 30% |
| | glacial acetic acid | 10% |

The gel was coated with a staining solution, stained at 50° C. for 30 min. with gentle shaking, and then destained under the same conditions. The destainer was changed several times. (The destainer can be regenerated by filtration over activated charcoal). When the desired decoloration was reached, the gel was rinsed with water, photographed (CCD video camera with “Easy” evaluation program and Thermoprinter, Herolab) and vacuum dried between two sheets (deti, Meckesheim) at 80° C.

c) Protein Concentration Determination

i. Photometric Determination

The protein concentration of the purified protein was determined as described (Stoscheck C. M., 1990) at OD₂₈₀ nm. In this respect the following formula holds:
protein concentration(mg/ml)=OD₂₈₀×MW/ε_{M},
where MW stands for the molecular weight; and ε_{M}, the molar extinction coefficient. For the proteins researched in this study, the protein-dependent multiplication factor
   P=MW/ε_{M} amounts to:
   CanA=19930.38/22900=0.87
   CanB=15606.44/7680=2.03
   CanC=16699.81/15990=1.04

ii. Bicinchonic Acid Test (BCA)

The test was conducted according to the manufacturer's guide (Sigma, Deisenhofen). To this end, aliquots of protein samples (CanA, B, C) and of known BSA dilutions were mixed with 50 times the volume of a fresh BCA/CuSO₄ (50:1) solution, incubated at 60° C. for 30 min. and measured in the spectrometer at 562 nm after cooling to RT. The protein concentrations were measured with the BSA calibration line.

iii. Amido Black Test (Heil and Zillig, 1970)

1-5 μl protein solution (Py-PP1) and 0.5-10 μg standard (BSA) were transferred to a cellulose acetate sheet (CA 251/0, Schleicher & Schuell, Dassel). After drying, the sheet was stained in 0.25% (w/v) amido black, 45% (v/v) methanol, 10% (v/v) glacial acetic acid for 10 minutes followed by being destained in 45% (v/v) methanol and 10% (v/v) glacial acetic acid. The sheet was dried again, protein spots were punched out and dissolved in 800 μl 10% (w/v) TCA, 80% (v/v) formic acid, 10% (v/v) glacial acetic acid respectively. Finally the OD₆₂₃ was measured; and the quantity of protein in the samples was determined by comparing with the BSA calibration line.

### Example 18

### Evaluation Of DNA And Protein Sequences

The analysis of the obtained DNA and protein sequences, homology calculations and the search for related sequences in the gene banks were performed with the program package from the University of Wisconsin Genetics Computer Group (UWGCG). To search for homologous DNA or protein sequences, the database of EBI, Hinxton Hall, UK was used. For example, the search programs “Fasta3,” “Blast2” and “Blitz” were used.

### Example 19

### Reconstitution Experiments

a) Protocol

The reconstitution experiments with the purified recombinant cannulae subunits were conducted in a 1.5 ml ERV. The batch volume was 50 μl. Aliquots of a newly thawed, purified protein (CanA: 1.3 mg/ml; CanB: 1.1 mg/ml; CanC: 2.0 mg/ml) were used. The different salt concentrations were adjusted by adding 1 M stock solutions of the appropriate chloride salts. Usually, 20 mM salt was added. The respective pH value was adjusted with HCl or NaOH. Then the pH value was estimated with pH indicator rods from Merck (Darmstadt).

Experiments under various temperatures between 4° C. and 100° C. were carried out. To prevent the batches from evaporating prematurely, they were coated with mineral oil. The reconstitution batches were incubated between 2 hours and 14 days and routinely checked for recombinant cannulae with the electron microscope. The standard incubation period was two days.

Standard batch at 30° C. (pH 6.0):

| | | |
|---|---|---|
| protein solution | 47 | μl |
| CaCl₂/MgCl₂ (per 1 M) | 1 | μl |
| HCl (2.5%) | 1 | μl |
| NaN₃ (0.1 M) | 1 | μl |

b) Evaluation

8 μl of each of the reconstitution batches were pipetted onto a mica-coated copper net (Plasma Cleaner PDC-3XG, Harrick Sci. Co., Ossinining, N.Y., USA) with carbon sheet (400 mesh, Taab, Berkshire, UK). After an absorption period of 15 seconds, the suspension was drawn off with filter paper from the bottom. After washed with a drop of H₂O_{bidist}, the grid was coated with a drop of 3% uranyl acetate solution. Then after waiting for 45 seconds, the contrast agent uranyl acetate was stripped away with filter paper. Then the preparation was analyzed with a Philips CM 12 transmission electron microscope (Philips, Eindhoven, NL).

c) Stability Experiments

The polymerized cannulae from CanA were checked for thermostability under different conditions. The stability experiments of the recombinant cannulae were conducted either in SME 1/2 or in standard polymerization buffer. To study the pressure dependence, excess pressure of 5 bar was adjusted, where stated, with N2 at room temperature. The batches were immersed either in the glycerol bath (F6-B5 model, Haake, Karlsruhe), or incubated in the hot air incubator (Heraeus, Hanau).

Buffers that were used:

The following solutions were established for the experiments after the polymerization of recombinant subunits.
standard polymerization buffer:
   50 mM Tris/HCl (pH 6.0), 80 mM NaCl, 9% glycerol, 20 mM CaCl₂, 20 mM MgCl₂
SME 1/2*: SME medium (see Example 6) 1:100 diluted with standard polymerization buffer

Following incubation, the diluted batches were collected by centrifugation at 20,000 rpm (JA 21 rotor) for 15 minutes. The pellet was absorbed in 10 μl standard polymerization buffer, with which the copper net was coated (see Example 24.b).

Incubation Vessels:
1.5 ml Eppendorf screw-cap reaction vessels with packing ring, during incubation without pressure.
Glass vessel with rounded edge, plugged with a rubber stopper and sealed with aluminum caps, during incubation with pressure (RT: 5 bar N₂)

The batches in the ERV were submerged directly into hot (100-130° C.) glycerol (60 min) and then cooled on ice. The batches in the vessels with rounded edge were put directly into the hot air incubator (90-140° C.) (75 or 95 min.). In the case of immersion in hot glycerol (60 min), they were pre-incubated (in glycerol) at 100° C. for 1 minute.

### Example 20

Production of the Polymer of the Present Invention.

a) 300L Fermentor Culture of Recombinant E. coli.

A 300 L culture of recombinant E. coli BL21 (DE3) harboring expression plasmid pEX-CAN-A (produced by attaching sequence substantially identical to SEQ ID NO. 1 to a vector pET17b using a procedure described in Example 20) was grown in a HTE-Fermentor (Bioengineering, Wald, Switzerland) at 37° C. under aeration (165 L air/min.) and stirring (400 rpm) with a doubling time of about 40 min. At an O.D. (600 nm) of 0.80, production of Can A protein was induced by addition of 30 grams of IPTG. Cells were harvested 3 hours after the induction and after being cooled down to 4° C. Cell yield: 1,610 grams (wet weight).

b) Production of the Polymer.

i. French Press.

250 g frozen cell mass of recombinant E. coli (stored at −60° C.) were suspended in 600 ml buffer (Tris-HCL 50 mM, pH 7.5, containing 80 mM NaCl and 9% (v/v) glycerol). Final volume: 900 ml. Cells were broken down by a French Press (Aminco; 1×20,000 PSI). The viscosity of the solution was lowered by shearing the DNA using an Ultraturrax blender and by adding additional 400 ml buffer.

ii. Centrifugation.

Particles were removed by centrifugation (Sorvall SS34 rotor; 19,000 rpm, 15 min.) and a clear supernatant (called “crude extract”) was obtained.

iii. Heat Precipitation.

To precipitate the heat-sensitive protein, the crude extract was heated to 100 C for 1 min. For example, the crude extract (1,200 ml) was pumped through a 75 cm long plastic hose (inner diameter, 5 mm; 4.75 ml/min) immersed in a 100° C. hot water-glycerol-bath (water: glycerol=1:1). The outlet end of the plastic hose was passed through an ice bath to cool down the solution in the hose before solution was finally collected using an Erlenmeyer flask.

iv. Centrifugation.

The heat-treated crude extract was centrifuged for 25 min. at 9,000 rpm in Sorvall rotor GSA. The clear supernatant was collected.

v. Ammonium sulfate Precipitation.

To the clear supernatant (840 ml), a 100% saturated ammonium sulfate solution (452 ml) was added at 4° C. (final ammonium sulfate concentration: 35% saturation). After 2 hours at 4° C., the precipitate was collected by centrifugation (1 hour; 13,000 rpm; Sowall rotor GSA). The precipitate was then solubilized in a buffer solution (final volume 171 ml; 12,35 mg protein/ml; 2,112 mg total protein) to form a protein solution. Finally, the protein solution was dialyzed by Rapid Dialysis against another buffer solution until its conductivity was the same as that of the buffer (3 hours).

vi. Polymerization.

The dialyzed protein solution was diluted by addition of buffer to a final protein concentration of 6.5 mg/ml (final volume 325 ml). Then, under shaking in a 1 L Erlenmeyer flask at 100° C. (in a water bath), the diluted protein solution was rapidly heated to 80° C. and then immediately transferred into a 500 ml screw-capped storage bottle. The storage bottle contained 3.32 ml (21.58 mg protein) of “Polymer Primers” (the “Polymer Primers” had been prepared before by 4 times French Press-shearing of a prefabricated Polymer suspension). Then, CaCl and MgCl (each at 20 mM final concentration) were added to the mixture and the closed bottle was stored in an 60° C. water bath. After addition of these salts, the solution became immediately turbid, indicating rapid polymerization of the protein units. After 10 min polymerization, the formed Polymer fibers were sheared by ultraturraxing the solution for 20 seconds in order to create additional polymer primers to speed up polymerization. Traces of silicone antifoam may be added before the ultraturraxing to reduce foaming. Typically, after 10 min. polymerization at 80° C., Polymer or polymer fibers could be observed under an electron microscope. After 1 to 2 hours of polymerization, protein polymers could be completely removed from the solution by centrifugation (15 min., 20,000 rpm, Sorvall rotor SS34), indicating complete polymerization.

Yield of polymer: 2.1 grams (protein) from 250 grams (wet weight) of E. coli (about 1 g Polymer (dry weight)/l 19 g E. coli).

vii. Storage.

Wet: At 4° C. in a buffer containing 10 mM Na-Azide.

Dry: Freeze-drying the polymer after the polymer being washed with an 1/10 diluted buffer followed by centrifugation.

c) Properties of Polymer Fiber

The polymer may have a shape of a short fiber, and therefore is also called “polymer fiber.” The polymer fiber is made from monomeric protein units (e.g. Can A: 182 amino acids: MW=19,830 Daltons, having a sequence of SED ID NO. 2). The secondary structure of the protein may be mainly P-sheets.

The protein subunits in the polymer are arranged in a right-handed or left-handed, two-stranded helix. Occasionally, the polymer fibers made up of a three-handed helix may be observed. The periodicity (the distance of one helix turn to the next) of the polymer is 4.4 nm.

The polymer has a unique quaternary structure. There is no similar protein complex known today among prokaryotes and eukaryotes. The polymer fiber has an outer diameter of 25 nm and inner diameter, 21 nm (in suspension). Under an electronic microscope, the dry negatively stained polymer fibres exhibit an outer diameter of 32 nm due to collapsing. Length of the polymer fiber is mostly between 3 and 5 micrometers. Some of the polymer fibers may reach a length from 10 to 25 micrometers.

The polymer fibers may form bundles of tens and hundreds of Polymer fibers with an overall diameter of 100 to 500 nm. Occasionally the bundle may reach an overall diameter of 4,000 nm. The polymer fiber is at least stable up to 128° C.

### Example 21

### Preparation of Lipid Coated Drug Delivery Complexes

To a solution containing 3 mg/ml monomeric protein units (e.g. Can A: 182 amino acids: MW=19,830 Daltons, having a sequence of SED ID NO. 2), a desired amount of drug molecules, and a sufficient amount of electrically neutral lipids, millimolar calcium and magnesium cations are added to form a mixture. The mixture is kept at ambient condition for a sufficient amount time until liposomes form. Thereafter, gel filtration chromatography is carried out on the mixture and the liposomes contained in the mixture are size fractionated. The desired fractions of the liposomes are then heated to 50° C. in the presence of millimolar amounts of calcium and magnesium cations to initiate the polymerization of the monomeric polypeptide units within each liposome. The polymerization results in the extreme deformation of the liposomes and produces sealed lipid tubules containing the drug molecules.

### Example 22

### Isolating Recombinant Proteins from E. coli

The following example describes an exemplary assay to isolate recombinant “cannulae” or “can” proteins from E. coli.

All exemplary assays in this example used:
Low salt buffer: 80 mMNaCl, 50 mM TRIS® (hydroxymethyl amino methane hydrochloride) (pH 7.5), 9% glycerol
High salt buffer: 1.2 M NaCl, 50 mM TRIS® (hydroxymethyl amino methane hydrochloride) (pH 7.5), 9% glycerol

Bicinchonic Acid Test (BCA): The test was conducted according to the manufacturer's guide (Sigma, Deisenhofen). To this end, aliquots of protein samples (CanA, B, C) and of known BSA dilutions were mixed with 50 times the volume of a fresh BCA/CuSO4 (50:1) solution, incubated at 60° C. for 30 min. and measured in the spectrometer at 562 nm after cooling to RT. The protein concentrations were measured with the BSA calibration line.

a) CanA and CanB

One gram of recombinant E. coli with a particular sequence such as CanA or CanB expressed was absorbed in 4 ml low salt buffer. Cell lysis was conducted with a French press (2×at 20,000 psi, American Instrument Co., Silver Spring, USA). After pelletizing the cell fragments (Eppendorf centrifuge, 13,000 rpm, 5 min., RT), the protein solution was incubated at 80° C. for 20 min. Then the denatured proteins were removed by centrifugation (as above). The supernatant was passed at 1 ml/min through a Q Sepharose column (1×12 cm=9.4 ml, Pharmacia, Freiburg). The eluent containing CanA or CanB was collected. The collected eluant was treated with leupeptin (1 μg/μl) and concentrated by a factor of 3-4 (based on the volume) in 4-8 hours in the MACROSEP™ centrifuge concentrators (Pall Filtron, Dreieich) with an exclusion limit of 5 kDA. After determining the protein concentration with the BCA test, the purified protein was shock frozen in liquid nitrogen in 100-200 μl aliquots and stored at −80° C. In each working step, a sample was taken and analyzed on an SDS polyacrylamide gel.

b) CanC

The first step of isolating CanC is same as that of CanA and CanB (see example 22.a). However, during the second step, CanC was retained on the Q sepharose. After flushing the column with low salt buffer, CanC was eluted from the column with a salt gradient (80-750 mM, in 60 ml) and collected by fractionation (1 ml each). Following analysis of the individual fractions on an SDS polyacrylamide gel, the CanC-containing fractions were combined and dialyzed against the low salt buffer at 4° C. overnight. Finally the protein solution was eluded at 1 ml/min through a 1 ml RESOURCEQ™ column (Pharmacia, Freiburg). Then a salt gradient (80-750 mM, in 60 ml) was applied and 0.5 ml fractions were collected. After analysis of the same on an SDS polyacrylamide gel, the CanC-containing fractions were combined again and dialyzed against low salt buffer overnight. Following addition of leupeptin (1 μg/μl), the solution was concentrated by a factor of 7 (based on the volume) in 6 hours in the MICROSEP™ centrifuge concentrators (Pall Filtron, Dreieich) with an exclusion limit of 5 kDa.

### Example 23

### Production of a CanA Polymer

The following example describes an exemplary protocol to produce a CanA polymer, including a chimeric polypeptide of the invention.

a) 300 L Fermentor Culture of Recombinant E. Coli.

A 300 L culture of recombinant E. coli BL21 (DE3) harboring expression plasmid pEX-CAN-A (produced by attaching sequence substantially identical to SEQ ID NO. I to a vector pETI 7b) was grown in a HTE-Fermentor (Bioengineering, Wald, Switzerland) at 37° C. under aeration (165 L air/min.) and stirring (400 rpm) with a doubling time of about 40 min. At an O.D. (600 nm) of 0.80, production of Can A protein was induced by addition of 30 grams of IPTG. Cells were harvested 3 hours after the induction and after being cooled down to 4° C. Cell yield: 1,610 grams (wet weight).

b) Production of the Polymer.

i. French Press: 250 g frozen cell mass of recombinant E. Coli (stored at −60° C.) were suspended in 600 ml buffer (TRIS® (hydroxymethyl amino methane hydrochloride) 50 mM, pH 7.5, containing 80 mM NaCl and 9% (v/v) glycerol). Final volume: 900 ml. Cells were broken down by a French Press (Aminco; 1×20,000 PSI). The viscosity of the solution was lowered by shearing the DNA using an Ultraturrax blender and by adding additional 400 ml buffer.

ii. Centrifugation: Particles were removed by centrifugation (Sorvall SS34 rotor; 19,000 rpm, 15 min.) and a clear supernatant (called “crude extract”) was obtained.

iii. Heat Precipitation: To precipitate the heat-sensitive protein, the crude extract was heated to 100° C. for 1 min. For example, the crude extract (1,200 ml) was pumped through a 75 cm long plastic hose (inner diameter, 5 mm; 4.75 ml/min) immersed in a 100° C. hot water-glycerol-bath (water: glycerol=1:1). The outlet end of the plastic hose was passed through an ice bath to cool down the solution in the hose before solution was finally collected using an Erlenmeyer flask.

iv. Centrifugation: The heat-treated crude extract was centrifuged for 25 min. at 9,000 rpm in Sorvall rotor GSA. The clear supernatant was collected.

v. Ammonium sulfate Precipitation: To the clear supernatant (840 ml), a 100% saturated ammonium sulfate solution (452 ml) was added at 4° C. (final ammonium sulfate concentration: 35% saturation). After 2 hours at 4° C., the precipitate was collected by centrifugation (1 hour; 13,000 rpm; Sowall rotor GSA). The precipitate was then solubilized in a buffer solution (final volume 171 ml; 12,35 mg protein/ml; 2,112 mg total protein) to form a protein solution. Finally, the protein solution was dialyzed by Rapid Dialysis against another buffer solution until its conductivity was the same as that of the buffer (3 hours).

vi. Polymerization: The dialyzed protein solution was diluted by addition of buffer to a final protein concentration of 6.5 mg/ml (final volume 325 ml). Then, under shaking in a 1 L Erlenmeyer flask at 100° C. (in a water bath), the diluted protein solution was rapidly heated to 80° C. and then immediately transferred into a 500 ml screw-capped storage bottle. The storage bottle contained 3.32 ml (21.58 mg protein) of “Polymer Primers” (the “Polymer Primers” had been prepared before by 4 times French Press-shearing of a prefabricated Polymer suspension). Then, CaCl and MgCl (each at 20 mM final concentration) were added to the mixture and the closed bottle was stored in an 60° C. water bath. After addition of these salts, the solution became immediately turbid, indicating rapid polymerization of the protein units. After 10 min polymerization, the formed Polymer fibers were sheared by ultraturraxing the solution for 20 seconds in order to create additional polymer primers to speed up polymerization. Traces of silicone antifoam may be added before the ultraturraxing to reduce foaming. Typically, after 10 min. polymerization at 80° C., Polymer or polymer fibers could be observed under an electron microscope. After 1 to 2 hours of polymerization, protein polymers could be completely removed from the solution by centrifugation (15 min., 20,000 rpm, Sorvall rotor SS34), indicating complete polymerization.

Yield of polymer: 2.1 grams (protein) from 250 grams (wet weight) of E. coli (about 1 g Polymer (dry weight)/119 g E. coli).

vii. Storage: Wet: At 4° C. in a buffer containing 10 mM Na-Azide. Dry: Freeze-drying the polymer after the polymer being washed with an 1/10 diluted buffer followed by centrifugation.

### Example 24

### Preparation of Lipid Coated Drug Delivery Complexes

The following example describes an exemplary protocol to prepare lipid coated drug delivery complexes comprising CanA, such as the chimeric polypeptides of the invention.

To a solution containing 3 mg/ml monomeric protein units (e.g. Can A: 182 amino acids: MW=19,830 daltons, having a sequence of SEQ ID NO. 2), a desired amount of drug molecules, and a sufficient amount of electrically neutral lipids, millimolar calcium and magnesium cations are added to form a mixture. The mixture is kept at ambient condition for a sufficient amount time until liposomes form. Thereafter, gel filtration chromatography is carried out on the mixture and the liposomes contained in the mixture are size fractionated. The desired fractions of the liposomes are then heated to 50° C. in the presence of millimolar amounts of calcium and magnesium cations to initiate the polymerization of the monomeric polypeptide units within each liposome. The polymerization results in the extreme deformation of the liposomes and produces sealed lipid tubules containing the drug molecules.

A number of embodiments of the invention have been described. Nevertheless, it will be understood that various modifications may be made without departing from the spirit and scope of the invention. Accordingly, other embodiments are within the scope of the following claims.

1. A chimeric polypeptide comprising at least a first domain comprising a cannulae polypeptide and a second domain comprising a heterologous polypeptide or peptide.

2. The chimeric polypeptide of claim 1, wherein the heterologous polypeptide or peptide is inserted at the amino terminal end, the carboxy terminal end or internal to the cannulae polypeptide.

3. The chimeric polypeptide of claim 1, wherein the cannulae polypeptide comprises a protein having 70% sequence identity to SEQ ID NO:2, SEQ ID NO:4, SEQ ID NO:6, SEQ ID NO:8, or SEQ ID NO:10, or a subsequence thereof, and is capable of assembling into a polymer, or, is capable of acting as a chiral selector.

4. The chimeric polypeptide of claim 3, wherein the cannulae polypeptide comprises a protein having 80% sequence identity to SEQ ID NO:2, SEQ ID NO:4, SEQ ID NO:6, SEQ ID NO:8, or SEQ ID NO:10, and is capable of assembling into a polymer.

5. The chimeric polypeptide of claim 4, wherein the cannulae polypeptide comprises a protein having 90% sequence identity to SEQ ID NO:2, SEQ ID NO:4, SEQ ID NO:6, SEQ ID NO:8, or SEQ ID NO:10, and is capable of assembling into a polymer.

6. The chimeric polypeptide of claim 5, wherein the cannulae polypeptide comprises a protein having 95% sequence identity to SEQ ID NO:2, SEQ ID NO:4, SEQ ID NO:6, SEQ ID NO:8, or SEQ ID NO:10, and is capable of assembling into a polymer.

7. The chimeric polypeptide of claim 6, wherein the cannulae polypeptide comprises a protein having sequence as set forth in SEQ ID NO:2, SEQ ID NO:4, SEQ ID NO:6, SEQ ID NO:8, or SEQ ID NO:10.

8. The chimeric polypeptide of claim 1, wherein the cannulae polypeptide is capable of assembling into a polymer.

9. The chimeric polypeptide of claim 8, wherein the cannulae polypeptide is capable of assembling into a nanotubule.

10. The chimeric polypeptide of claim 9, wherein the heterologous polypeptide or peptide is expressed in the inner lumen of the nanotubule.

11. The chimeric polypeptide of claim 9, wherein the heterologous polypeptide or peptide is expressed on the exterior of the nanotubule.

12. The chimeric polypeptide of claim 1, wherein the heterologous polypeptide or peptide comprises a chiral selection motif.

13. The chimeric polypeptide of claim 1, wherein the heterologous polypeptide or peptide comprises a receptor or a ligand.

14. The chimeric polypeptide of claim 1, wherein the heterologous polypeptide or peptide comprises an enzyme.

15. The chimeric polypeptide of claim 14, wherein the heterologous polypeptide or peptide comprises an enzyme active site.

16. The chimeric polypeptide of claim 1, wherein the heterologous polypeptide or peptide comprises an antigen or an antigen binding site.

17. The chimeric polypeptide of claim 1, wherein the heterologous polypeptide or peptide comprises a green fluorescent protein, an alpha-galactosidase or a chloramphenicol acetyltransferase.

18. The chimeric polypeptide of claim 1, wherein the chimeric polypeptide is a recombinant protein.

19. The chimeric polypeptide of claim 1, wherein at least one subsequence of the cannulae polypeptide has been removed.

20. The chimeric polypeptide of claim 19, wherein the heterologous polypeptide or peptide is inserted into the cannulae polypeptide at the site the subsequence was removed.

21. The chimeric polypeptide of claim 19, wherein the cannulae polypeptide is a CanA polypeptide and the removed subsequence is a 14 residue motif consisting of residue 123 to residue 136 of SEQ ID NO:2 (PDKTGYTNTSIWVP), or, a 17 residue motif located at amino acid residue 123 to residue 139 of SEQ ID NO:2 (PDKTGYTNTSIWVPGEP).

22. The chimeric polypeptide of claim 21, wherein the heterologous polypeptide or peptide is inserted into the CanA polypeptide at the site the subsequence was removed.

23. The chimeric polypeptide of claim 22, wherein the heterologous polypeptide or peptide is a 14 or a 17 residue motif inserted into the CanA polypeptide to replace the removed 14 or a 17 residue motif.

24. An immobilized chimeric polypeptide comprising the chimeric polypeptide of claim 1.

25. A nanotubule comprising a plurality of chimeric polypeptides as set forth in claim 1.

26. The nanotubule of claim 25, wherein the heterologous polypeptide or peptide comprises an enzyme.

27. The nanotubule of claim 26, wherein the nanotubule comprises a plurality of different enzymes.

28. The nanotubule of claim 27, wherein the plurality of enzymes comprising a biosynthetic pathway.

29. The nanotubule of claim 28, wherein the plurality of enzymes are arranged along the length of the nanotubule in the same order as they act in the biosynthetic pathway.

30. The nanotubule of claim 25, wherein the heterologous polypeptide or peptide comprises a chiral selection motif.

31. A nucleic acid comprising a sequence encoding the chimeric polypeptide of claim 1.

32. An expression cassette comprising the nucleic acid of claim 31.

33. A cell comprising the nucleic acid of claim 31.

34. The cell of claim 33, wherein the cell is a bacterial cell, a plant cell, a yeast cell, a fungal cell, an insect cell or a mammalian cell.

35. A transgenic non-human animal comprising the nucleic acid of claim 31.

36. A plant comprising the nucleic acid of claim 31.

37. A method for the chiral selection of a composition, comprising the following steps:
(a) providing a chimeric polypeptide as set forth in claim 12;
(b) providing a racemic mixture of the composition; and,
(c) contacting the racemic mixture with the chimeric polypeptide under conditions wherein only one enantiomer of the composition binds to the chimeric polypeptide; thereby selecting a single chiral specie of the racemic mixture.

38. A method for the chiral selection of a composition, comprising the following steps:
(a) providing a nanotubule as set forth in claim 29;
(b) providing a racemic mixture of the composition; and,
(c) contacting the racemic mixture with the nanotubule under conditions wherein only one enantiomer of the composition binds to the nanotubule; thereby selecting a single chiral specie of the racemic mixture.

39. A method for enzymatic biosynthesis of a composition, comprising the following steps:
(a) providing a nanotubule comprising a plurality of enzymes comprising a biosynthetic pathway as set forth in claim 28;
(b) providing a substrate for at least one enzyme; and,
(c) contacting the nanotubule with the substrate under conditions wherein the enzymes of the biosynthetic pathway catalyze the synthesis of the composition.

40. The method of claim 39, wherein the enzymes are expressed in the inner lumen of the nanotubule.

41. The method of claim 39, wherein the enzymes are expressed on the exterior of the nanotubule.

42. A drug delivery system comprising: a polymeric encapsulation medium made by self-assembly of a plurality of polypeptides; and at least one drug encapsulated in said polymeric encapsulation medium.

43. The drug delivery system of claim 42 further comprising a targeting vector.

44. The drug delivery system of claim 42, wherein the polypeptide has a sequence selected from the group consisting of SEQ ID NOS: 2, 4, 6, 8 and 10, and sequences having at least 70% sequence identity to a sequence selected from SEQ ID NOS: 2, 4, 6, 8 and 10, or a subsequence thereof, as determined by analysis with a sequence comparison algorithm or by visual inspection.

45. A method of producing a polypeptide polymer by self-assembly comprising the steps of: providing a plurality of polypeptides capable of self-assembly in the presence of a divalent cation; and polymerizing the polypeptides in the presence of a divalent cation and a template molecule.

46. The method of claim 45, further comprising adding a template molecule and alkaline earth metal ions to the solution.

47. A method of delivering a drug to a location in a human or an animal body comprising the step of: administering a drug delivery system as set forth in claim 42 to a human or animal body.

48. The method of claim 47 further comprising the step of releasing the drug from the delivery system at the location in the human or animal body.

49. The method of claim 47 further comprising the steps of: dissolving the plurality of polypeptides and the drug in a solution; and, polymerizing the plurality of polypeptides in the presence of the drug so as to encapsulate the drug in the polymer to form the drug delivery system.

50. A nanorobot comprising polypeptides having a sequence selected from the group consisting of SEQ ID NO:2, SEQ ID NO:4, SEQ ID NO:6, SEQ ID NO:8, or SEQ ID NO:10, and sequences having at least 70% sequence identity to a sequence selected from SEQ ID NO:2, SEQ ID NO:4, SEQ ID NO:6, SEQ ID NO:8, or SEQ ID NO:10, or a subsequence thereof.

51. A biochip comprising polypeptides having a sequence selected from the group consisting of SEQ ID NO:2, SEQ ID NO:4, SEQ ID NO:6, SEQ ID NO:8, or SEQ ID NO:10, and sequences having at least 70% sequence identity to a sequence selected from SEQ ID NO:2, SEQ ID NO:4, SEQ ID NO:6, SEQ ID NO:8, or SEQ ID NO:10, or a subsequence thereof.

52. A fiber comprising polypeptides having a sequence selected from the group consisting of SEQ ID NO:2, SEQ ID NO:4, SEQ ID NO:6, SEQ ID NO:8, or SEQ ID NO:10, and sequences having at least 70% sequence identity to a sequence selected from SEQ ID NO:2, SEQ ID NO:4, SEQ ID NO:6, SEQ ID NO:8, or SEQ ID NO:10, or a subsequence thereof.

53. A liposome comprising polypeptides having a sequence selected from the group consisting of SEQ ID NO:2, SEQ ID NO:4, SEQ ID NO:6, SEQ ID NO:8, or SEQ ID NO:10, and sequences having at least 70% sequence identity to a sequence selected from SEQ ID NO:2, SEQ ID NO:4, SEQ ID NO:6, SEQ ID NO:8, or SEQ ID NO:10, or a subsequence thereof.
